(19) United States
(12) Patent Application Publication
(10) Pub. No.: US 2003/0130799 A1
(43) Pub. Date: Jul. 10, 2003

(54) Structure/properties correlation with membrane affinity profile

(76) Inventors: Charles Pidgeon, West Lafayette, IN (US); Nadege Lagneau, Quincy, MA (US); Sonyuan Lin, Natick, MA (US); Jeffrey A. Ruell, Chelsea, MA (US)

Correspondence Address: BARNES & THORNBURG, 11 SOUTH MERIDIAN, INDIANAPOLIS, IN, 46204

(21) Appl. No.: 10/117,160

(22) Filed: Apr. 5, 2002

Related U.S. Application Data
(60) Provisional application No. 60/281,749, filed on Apr. 5, 2001.

(51) Int. Cl. G06G 7/48; G06G 7/58; G06F 19/00; G01N 33/48; G01N 33/50

(52) U.S. Cl. 702/19; 703/11

(57) ABSTRACT

A method and system for implementing discovery research is described. Pharmacokinetic and other relevant properties of proposed biologically active drug substances are estimated based at least in part on empirically defined correlations of structures/properties of biologically effective known substances with their respective affinities for multiple membrane mimetic surfaces. Computer implemented structure similarity or substructure searching coupled with use of a database including compound structures values corresponding to drug affinities for membrane mimetic surfaces, and pharmacokinetic data provides a powerful tool for discovery research.

### CROSS REFERENCE TO RELATED APPLICATION

This application claims priority under 35 U.S.C. § 119(e) to U.S. Provisional Application No. 60/281,749, filed Apr. 5, 2001 expressly incorporated by reference herein.

### FIELD OF INVENTION

This invention relates to discovery research. It is directed to predicting biologically relevant properties for discovery research compounds using data sets/calibration curves derived at least in part from empirical membrane or membrane mimetic binding data for known biologically effective substances. More particularly, the invention is directed to methods and systems for carrying out discovery research for using a data set including arrays of structural and empirical information for known biologically effective substances.

### BACKGROUND OF THE INVENTION

There continues to be a significant development effort directed to the development of research tools/protocols for enhancing the efficiency and efficacy of the discovery research for finding new biologically effective compounds such as drugs, herbicides, pesticides, and the like. The goal of such efforts has been to define efficient methodologies for predicting not only biological activity, but as well, the pharmacokinetic properties of compounds of proven systemic efficacy critical to their systemic efficacy. Thus, for example, in the past, drug leads have been generated by comparing their structural, biological and physical properties with those of known compounds having recognized biological activity in vivo. There has been developed a significant body of literature directed to the development of discovery protocols designed not only to predict in vitro biological activity, but as well to predict pharmacokinetic properties based on comparison of physical, chemical, and biological descriptors and the use of pattern recognition analysis of such descriptors.

With the advent of combinatorial chemistry and other high throughput techniques for identifying hit compounds, for example, compounds exhibiting in vitro receptor binding affinity predictive of biological activity, there continues to be a need for the development of high throughput methods and systems for predicting pharmacokinetic properties and providing guidance for design of new biologically active compounds to improve pharmacokinetic properties.

### SUMMARY OF THE INVENTION

The present invention is based at least in part on the discovery that known drug substances, necessarily exhibiting pharmacokinetic properties for therapeutic efficacy, exhibit affinities for various membrane mimetic surfaces at levels within a fairly well defined range, and that compound structures, the taxonomic classification of the endogenous target molecule, and the respective pharmacokinetic properties are all closely correlated with a drug's relative affinities for two or more membrane-like or membrane mimetic surfaces. While the teaching herein is made principally with respect to drug discovery research, it will be recognized that the principles, methods and systems described herein can be applied to discovery research for other biologically active compounds including pesticides, herbicides, and the like, in the respective biological systems in which they are used.

The present invention makes use of the discovery of a significant and surprising level of correlation between 1) empirically determined values relating to the level of affinity for two or more membrane or membrane mimetic surfaces, and 2) with both the chemical structure and the pharmacokinetic, and as well the pharmacodynamic properties of the compound exhibiting such relative membrane affinities. Thus important to implementation of the present invention is the preparation and utilization of a data set including structural and empirical information for known drug substances (and, with advantage, for other compounds as well) wherein the empirical data includes values relating to the affinity of the respective compound represented in the data set for two or more surfaces, typically membrane mimetic surfaces, and optionally, but preferably, values, numeric or otherwise, relating to known pharmacokinetic and pharmacodynamic properties, and the nature (i.e., the taxonomic classification) of the endogenous macromolecular target, if known, of the drug substances represented in the data set. The data set can be assembled as well to include art-recognized molecular descriptors, if known, for at least a portion of the drug compounds represented by the data set.

In one embodiment the data set is stored electronically in computer accessible form/format as an array of values associated with each compound member of the data set. The chemical structural data for the respective compounds can be stored in either a two-dimensional or three-dimensional format accessible and searchable using commercially available search software capable of identifying those chemical structures in the data set exhibiting some predefined degree of structural/substructural similarity with a test compound.

The numeric values characteristic of membrane affinity for use in forming the data set can be determined by any of a wide variety of art-accepted techniques. In one embodiment of this invention the numeric values characteristic of membrane affinity are determined chromatographically using an aqueous mobile phase and a stationary phase comprising a membrane mimetic surface, for example, in a high performance liquid chromatographic system such as that described in U.S. Pat. No. 4,931,498, expressly incorporated herein by reference. The term membrane or “membrane mimetic surface” as used in describing and defining the present invention, refers to any surface bearing amphiphilic molecules (i.e., those having both lipophilic and hydrophilic portions capable of exhibiting some selective affinity or otherwise interacting with a solute (for example, a test or control compound) in a fluid phase in contact with the surface. The amphiphilic molecules may be mobilized or immobilized in any device capable of providing empirically derived membrane affinity values. The term is intended to encompass a broad scope of commercially available stationary phases detailed for use in chromatographic applications. Preferred membrane mimetic surfaces are those described in the above-incorporated U.S. Pat. No. 4,931,948. They can be used to obtain values relating to membrane affinity for multiple compounds by the methods and equipment detailed in, for example, published PCT International Applications WO 01/88528 and WO 99/10522.

Thus, in its broadest scope, the present invention is directed to use of a data set comprising chemical structural and empirical data for known drug substances, i.e., compounds proven safe and effective for therapeutic use. The empirical information for the drug compound members in the data set typically includes a value characteristic of the relative affinity exhibited by the compound for at least two unique membrane or membrane mimetic surfaces, preferably at least one of which is a substantially neutral surface and the other of which is a negatively charged surface (under the conditions of measured membrane affinity). The data array for the drug compound members of the data set can, and preferably does, include values indicative of pharmacokinetic and/or pharmacodynamic properties of at least a portion of the drug members of the data set. In one embodiment the data set includes as well as value relating to the nature, more particularly the taxonomic classification, of the endogenous molecule known to be the target of the respective data set drug substance member.

The data set is used, typically stored electronically in a computer readable format and used in systems and methods for drug discovery research. Thus in one aspect of the invention the data set is used as a basis for predicting pharmacokinetic properties of a proposed drug substance or test compound, either of known or unknown chemical structure. Predictions are based on calibration curves that correlate membrane-binding properties with pharmacokinetic properties, e.g., volume of distribution, protein binding, or clearance. Membrane-binding properties can be either empirically measured or predicted from the compound's structure. Typically, the data set is built from compounds with known structures and empirically measured membrane binding data. If the structure of the test compound is known, the chemical structures in the data set can be searched using any one of several commercially available structure similarity search programs to identify the chemical structures of compounds in the data set exhibiting some predetermined degree of structural similarity with the test compound. The pharmacokinetic properties of the compound or compounds exhibiting the specified degree of similarity can be assigned as an estimate of the pharmacokinetic properties of the proposed drug substance/test compound.

Alternatively, if the structure of the test compound is not known, its relative affinities to at least two of the membrane mimetic surfaces, represented by the numeric values in the data set, can be measured empirically, for example, by chromatographic analysis, and the empirically determined membrane affinity values for the test compound or drug substance can be used as the basis of a search of the data set for data set drug compound members exhibiting similar membrane binding characteristics. The pharmacokinetic properties of the data set member exhibiting the most similar membrane binding characteristics can be assigned as an estimate of the pharmacokinetic properties of the test compound. Similar protocols can be followed to predict the taxonomic classification of potential endogenous macromolecular targets of the test compound. Such predictions based on structure can use calibration plots that are 2- or 3-(or more) dimensional, plots obtained by principle component analysis or other statistical methods known in the art.

In another embodiment of the invention making use of the inherent correlation of chemical structure and relative membrane affinity values reflected in the data set, there is provided a method for designing chemical structure modifications of a hit compound to produce proposed chemical structures for test drug substances having improved pharmacokinetic properties relative to those of the hit compound. The method includes the steps of obtaining numeric values relating to the affinity of the hit compound to at least two of the membrane mimetic surfaces. The method also required correlating the known pharmacokinetic properties of the control compounds, i.e., the data set drug substance members, with their respective positions on a plot of an array of numeric values relating to the affinities of the control compound to the respective membrane mimetic surfaces. The method also includes the step of correlating the respective positions of the drug compound data set members in the array with their respective chemical structures. Potential modifications of the chemical structure of the hit compound are identified based on such correlations to produce a predictable change in relative membrane binding affinities, and thus pharmacokinetic values, correlated therewith. The compound represented by the modified chemical structure is prepared and then assayed for target macromolecule binding activity and optionally membrane binding affinities.

In still another embodiment of the invention a membrane binding data set for known drug substances is used principally as a filter of sorts to eliminate, based on probable lack of pharmacokinetic properties required for drug efficacies, at least a portion of compounds identified in a first step of an in silico drug discovery protocol. The in silico drug discovery method is used for identifying drug candidates having both potential for binding to a macromolecule recognized to be an endogenous target of a set of known drug substances, and as well, favorable pharmacokinetic properties. The method comprises the step of identifying chemical structural or substructural characteristics of a set of said known drug substances and conducting a structure or substructure similarity search of a data base comprising chemical structures of commercially available compounds to identify those structures in a data base of compounds having a threshold predetermined degree of similarity to at least one of the known drug substances. The identified chemical structures are compared with those in the above-describe data set to eliminate those identified compounds based on their probable lack of pharmacokinetic properties required for drug efficacy judged by their predicted relative membrane binding affinities. Compounds not eliminated by such filtering step can then be tested to assess for binding affinity to the macromolecular target.

In one embodiment, the invention contains a data set comprising chromatographic data from 2 or more chromatography columns, biological data, and compound structural data. Art-recognized molecular descriptors, for example, molecular volume, molecular surface area, hydrophobicity, fractional polar surface area, hydrophilicity factor, number of donor atoms for hydrogen bonding, number of receptor atoms for hydrogen bonding, sum of atomic polarizability, etc., may also be included in the data set. A calibration curve is prepared from the chromatographic and/or molecular descriptor data. The biological data is predicted either from empirically determined chromatographic data or compound structure searches that find similar data-set compounds to the test compound. Effectively, the chromatographic data is predicted from the structural similarity search, which is used to predict the biological data. The chromatographic data, which may be indicative of membrane binding interactions may be measured by any method that characterizes the interaction between solutes and membranes. The structural searches may be either 2-dimensional or 3-dimensional and the search criteria may be unique to each biological property. Biological properties include, but are not limited to, taxonomy, receptor type, therapeutic indication, volume of distribution, protein binding and clearance.

In another embodiment the invention uses a searchable database to predict pharmacokinetic data from chromatographic data with or without co-use of molecular descriptors.

### BRIEF DESCRIPTION OF THE DRAWINGS

FIG. 1 is a graph presenting membrane affinity values forming at least a portion of a data set for use in drug discovery protocols in accordance with this invention. Each point on the graph represents the membrane affinity values for a known drug compound member of the data set used in accordance with this invention and represents the relative binding of said compound to a membrane mimetic surface comprising phosphatidyl ethanolamine (X-axis) and another membrane mimetic surface comprising phosphatidyl serine (Y-axis).

FIG. 2 is similar to FIG. 1 except that only drug substances exhibiting enzyme inhibition activity are represented.

FIG. 3 is similar to FIG. 1 except that only drug substances exhibiting activity through G-protein linked receptors are represented.

FIG. 4 is similar to FIG. 1 except that only drug substances interacting with intracellular receptors are represented.

FIG. 5 is similar to FIG. 1 except that only drug substances interacting with ion channels are represented.

FIG. 6 is similar to FIG. 1 except that only drug substances exhibiting ion transporter inhibition activity are represented.

FIG. 7 is similar to FIG. 1 except that only drug substances interacting with ligand-gated ion channels are represented.

FIG. 8 is similar to FIG. 1 except that only compounds exhibiting neurotransmittor re-uptake inhibition activity are represented.

FIG. 9 is similar to FIG. 1 except that only compounds exhibiting interaction with nucleic acids are represented.

FIG. 10 is similar to FIG. 1 except that only compounds exhibiting known protein binding levels are represented. The graph further indicated the known level of protein binding of the represented compounds within one of four subranges: <40%, 40-89%, 90-97%, and >97%.

FIG. 11 is similar to FIG. 1 except that only compound members of the data set having known volume of distribution are represented. The data points are further distinguishable as falling within one or four subranges: low (0-40 liters), moderate (41-69 liters), high (70-50 liters), and very high (>250 liters).

FIG. 12 is similar to FIG. 1 except that only members of the data set having known values for total body clearance are represented. The data points are further identified as following within one of two subranges: low (0-0.378 L/min) and high (>0.378 L/min).

FIG. 13 illustrates the zones of relative membrane affinities on a plot of Log κ′ values for a membrane mimetic surface comprising phosphatidyl serine (Y-axis) and a membrane mimetic surfaces comprising phosphatidyl ethanolamine (X-axis) for compounds known to target an endogenous macromolecule of the taxonomic classification labeling the various zones.

FIGS. 14 and 15 are similar to FIG. 1 illustrating the membrane affinity/structure relationship of the data set compounds relative to surfaces comprising phosphatidyl ethanolamine and phosphatidyl serine, respectively.

FIG. 16 is a PCA plot of four IAM κ′ values shows clustering for the main pharmacological taxonomies or efficacy mechanisms. Taxonomy classifications were made according to Morton (1997).

FIG. 17 is an enlargement of PCA plot of four IAM κ′ values showing the intra-compound relationship with a receptor classes.

FIG. 18 is a graph illustration the relationships between efficacy mechanism and two IAM parameters.

FIG. 19 is a graph showing volume of distribution as a function of two IAM parameters. The pharmacological properties for each drag were obtained from Goodman and Gilman's The Pharmacological Basis of Therapeutic Action, Vol. 9. Drugs were placed into one of four possible volume of distribution groups. These were “low” (0-40 L), “moderate” (41-69 L), “high” (70-250 L) or “very high” (greater than 250 L). A 70-kg patient was assumed in all cases.

FIG. 20 is a graph showing protein binding as a function of two IAM parameters. The pharmacological properties for each drug were obtained from Goodman and Gilman's The Pharmacological Basis of Therapeutic Action, Vol. 9. Drugs were divided into four groups: less than 40%, 40-90%, 90-98%, or greater than 98% bound to serum proteins. These values include all types of serum proteins and represent in vitro results.

FIG. 21 is a graph showing equilibrium brain to blood concentration differences of a drug as a function of ^{Ester}IAM.PS^{C10/C3} κ′. Log BB data is from either Calder (1997) or Abraham (1995).

FIG. 22 is a graph showing plasma to blood cell concentration ratio of drugs as function of ^{Ester}IAM.PS^{C10/C3} κ′. Data is from Hinderling (1997).

FIG. 23 is a graph showing the relationship between human oral absorption and 4 IAM κ′. Oral absorption values are from Irvine (1998).

FIG. 24 is the standard curve for Clearance used in Study 1. Each data point on the graph represents a drug with known clearance data. The pharmacological properties for each drug were obtained from Goodman & Gilman's “The Pharmacological Basis of Therapeutic Action”, Vol. 9. Clearance values (total body) were corrected for renal clearance by taking the reported total body clearance values and multiplying by the fraction not excreted in urine (hepatic fraction). Drugs were classified as “low” for compounds with clearance lower than 0.378L/min or “high” for those greater than 0.378 L/min. The standard curve was established with 80 compounds.

FIG. 25 is the standard curve for volume of distribution used in Study 1. Each data point on the graph represents a drug with known volume of distribution data. The pharmacological properties for each drug were obtained from Goodman & Gilman's “The Pharmacological Basis of Therapeutic Action”, Vol. 9. Volume of distribution values are normalized to a 70-kg patient. Drugs were placed into one of the following four possible volume of distribution groups: “low” (0-40L), “moderate” (41-60 L), “high” (70-250 L), or “very high” (>250 L). The standard curve was established with 80 compounds.

FIG. 26 is the standard curve for % protein binding used in Study 1. Each data point on the graph represents a drug with known % of protein binding data. The pharmacological properties for each drug were obtained from Goodman & Gilman's “The Pharmacological Basis of Therapeutic Action”, Vol. 9. The reported values are in vitro data, and represent the percentage of drug in the plasma that is bound to plasma proteins. Drugs were placed into one of the following four possible % protein binding groups: <40%, 40-89%, 90-97% or >97%. The standard curve was established with 80 compounds.

FIG. 27 depicts the chemical structures of the parent compound (Clonazepam) and derived chemical library of 7 structurally related compounds used in Study 1.

FIG. 28 shows the predicted positions of the 7 test compounds in Study 1 on the chemical space representing ˜400 known drugs or chemical entities of pharmacological relevance.

FIG. 29 depicts the 7 test compounds overlaid on the standard curve for clearance. This graph was used for predicting the clearance of the test compounds in Study 1.

FIG. 30 depicts the 7 test compounds overlaid on the standard curve for volume of distribution. This graph was used for predicting the volume of distribution of the test compounds in Study 1.

FIG. 31 depicts the 7 test compounds overlaid on the standard curve for % protein binding. This graph was used for predicting the % protein binding of the test compounds in Study 1.

FIG. 32 depicts the chemical structures of the parent compound (Atenolol) and derived chemical library of 11 structurally related compounds used in Study 2.

FIG. 33 shows the predicted positions of the 11 test compounds in Study 2 on the chemical space representing ˜400 known drugs or chemical entities.

FIG. 34 depicts the 11 test compounds overlaid on the standard curve for clearance. This graph was used for predicting the clearance of the test compounds in Study 2.

FIG. 35 depicts the 11 test compounds overlaid on the standard curve for volume of distribution. This graph was used for predicting the volume of distribution of the test compounds in Study 2.

FIG. 36 depicts the 11 test compounds overlaid on the standard curve for % protein binding. This graph was used for predicting the % protein binding of the test compounds in Study 2.

FIG. 37 is similar to FIG. 1 with the additional feature that the membrane binding data of Flurazepam and Diazepam are plotted on the graph.

FIG. 38 is similar to FIG. 1 with the additional feature that the membrane binding data of desmethyldiazepam and clorazepate are plotted on the graph.

FIG. 39 is similar to FIG. 1 with the additional feature that the membrane binding data of Nabumetone and Naproxen are plotted on the graph.

FIG. 40 is similar to FIG. 10 with the exception that the membrane binding constants of the test compound Ibuprofen is plotted on the graph. Membrane binding measurements on IAMs were performed on Ibuprofen, but not Ketoprofen, Naproxen and Flubiprofen, the structures of which are in an inset on the Figure.

FIG. 41 is the same plot as FIG. 40 with the exception that the predicted membrane binding data and therefore protein binding, of Ketoprofen, Naproxen, and Flubiprofen are added to the graph based on predictions using chemical principles of the expected effect of structural modification and solute retention.

FIG. 42 is the same plot as FIG. 41 with the exception that different test molecules were used to demonstrate that the protein binding of unknown compounds can be predicted from the calibration curve shown in FIG. 10. The test molecules are Nadolol, Propranol, Pindol, and Timolol.

FIG. 43 is similar to FIG. 11 with the exception that different test molecules were used to demonstrate that the volume of distribution can be predicted from the calibration curve shown in FIG. 9. The same compounds used for predicting protein binding in FIG. 42 were used for this Figure.

FIG. 44 is similar to FIG. 12 except clearance of the B-adrenergic compounds is being predicted using Propranol as the test compound and Nadolol, Pindolol, and Timolol as the test compounds.

FIG. 45 shows the results of using 4 structurally different test solutes (left column) as query molecules for searching either the ADME database or the MAF database. The results (hits) of the searches are shown in the middle column when the ADME database was searched and in the right column when the entire MAF database was searched.

FIG. 46 a and b show membrane binding properties predicted directly from the substructure query search of the ADME database (FIG. 46a) and the MAF database (FIG. 46b) for the test compounds shown in the left column of FIG. 45.

FIG. 47 depicts structures of compounds having SSRI activity.

FIGS. 48a and b depict structures of compounds found using substructure search of the Chem ACX-SC database.

FIG. 49 depicts structures of compounds found using substructure searching of the Chem ACX-Pro database.

FIG. 50 depicts the structure of citalopram and fluoxetine.

### DETAILED DESCRIPTION OF THE INVENTION

The present invention provides a powerful tool for drug discovery research. It not only enables the prediction of pharmacokinetic and pharmacodynamic properties, but it also enables the medicinal chemist to predict the nature of the endogenous target molecules with which a putative test drug compound is likely to interact. The predictions can be based on the chemical structure of the compound, if known, or on membrane affinity values determined empirically for the test compound. The data set used in carrying out this invention thus provides a means for correlating the chemical structures, pharmacokinetic values, and taxonomic classification of macromolecular targets with values for relative affinities of the data set compounds for two or more membrane mimetic surfaces. Preferably the data set includes values relating to the affinity of the respective data set members for at least one substantially neutral membrane mimetic surface and another exhibiting a negative charge at physiological pH.

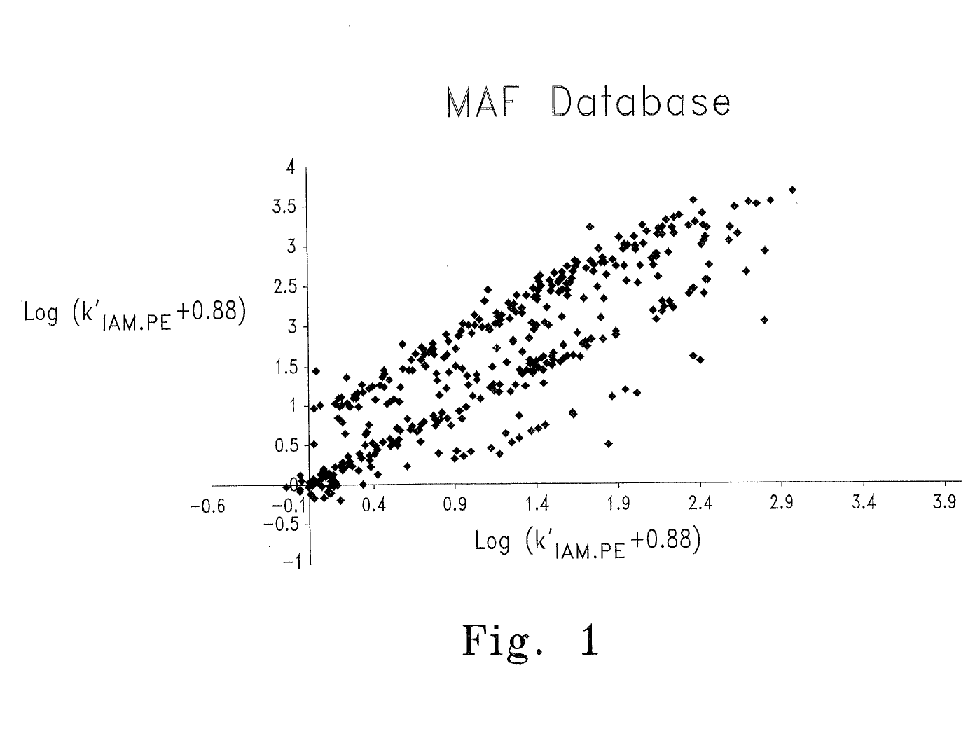

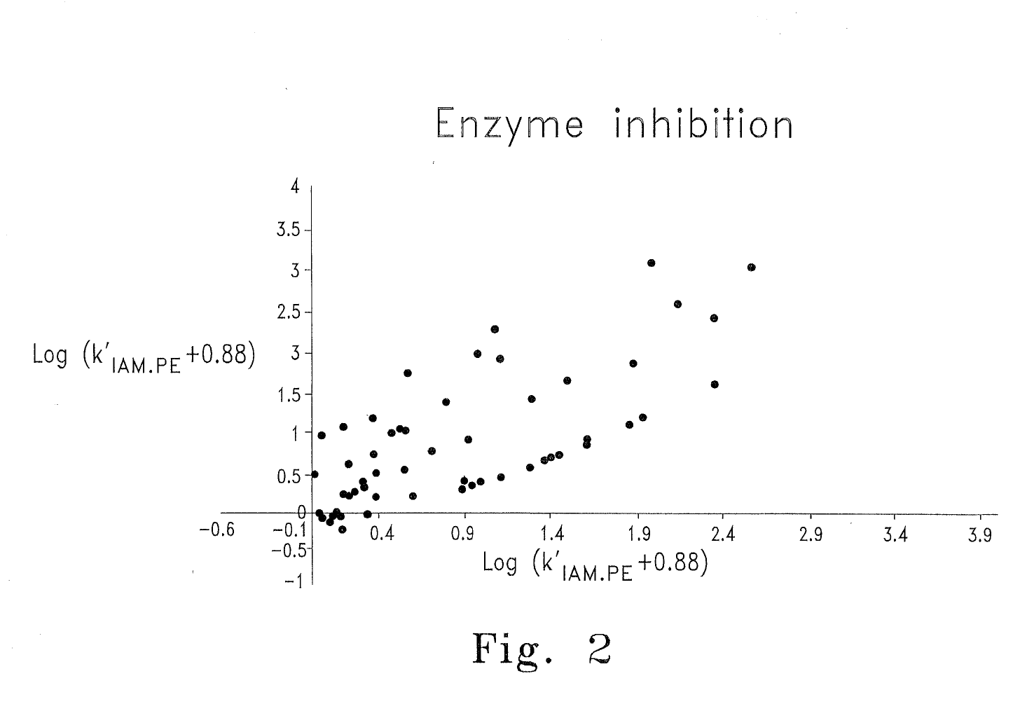

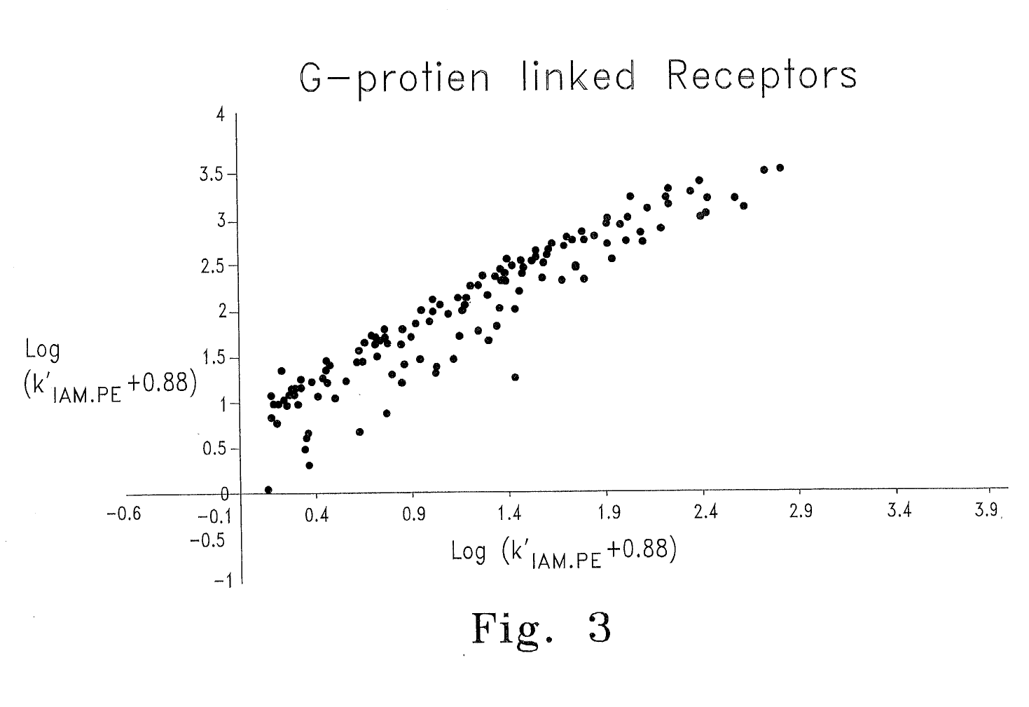

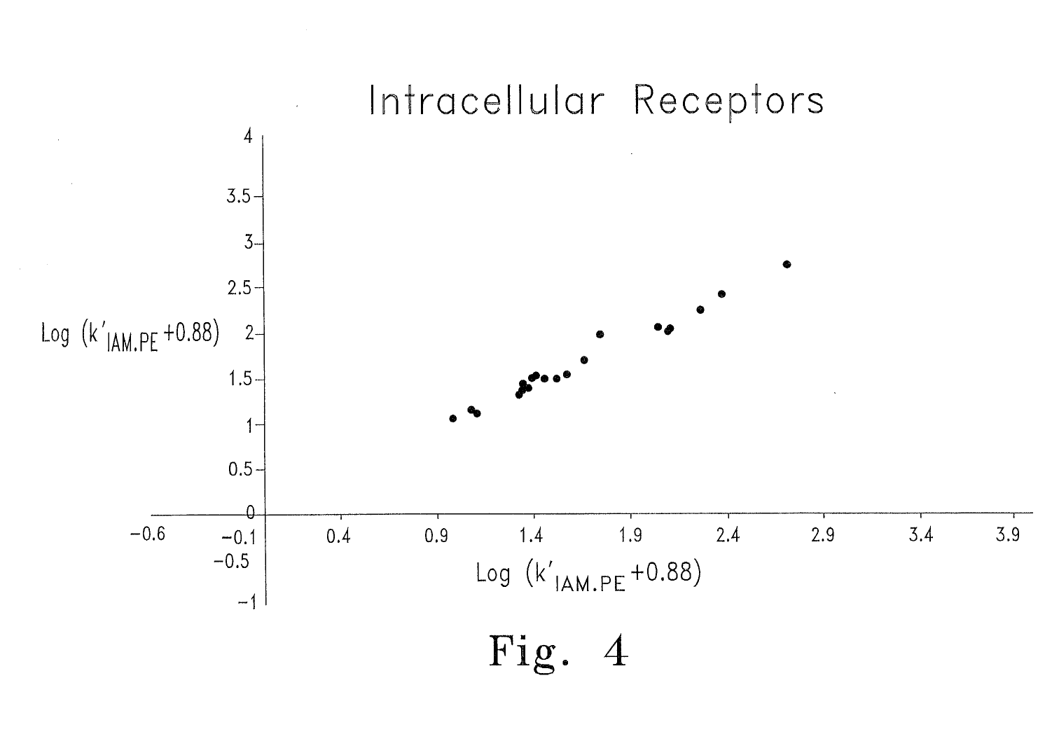

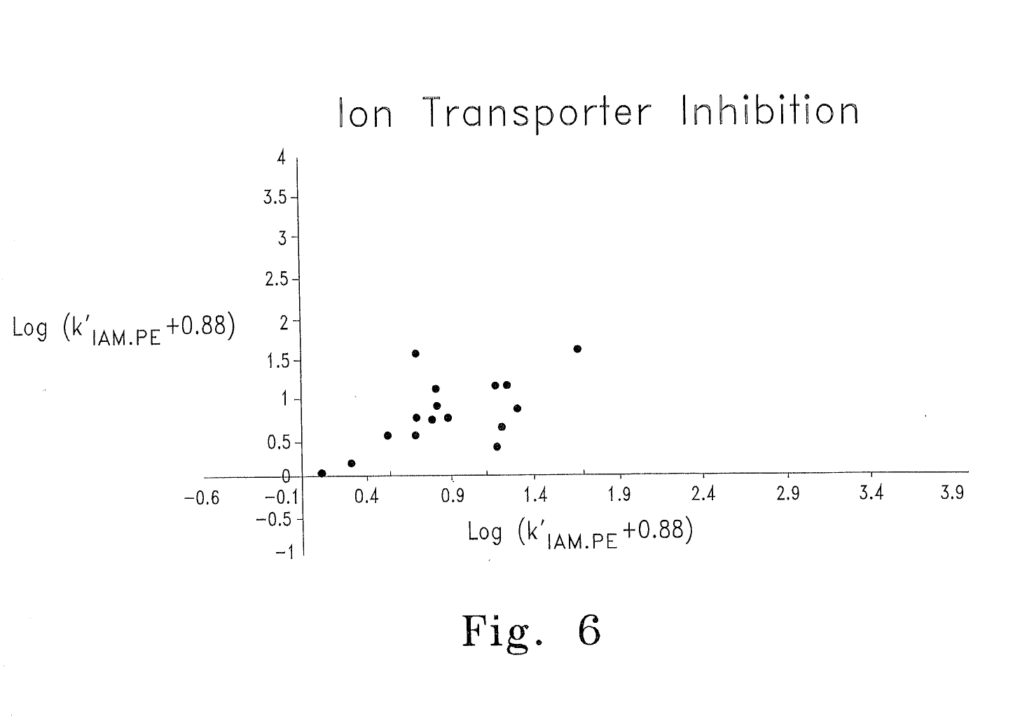

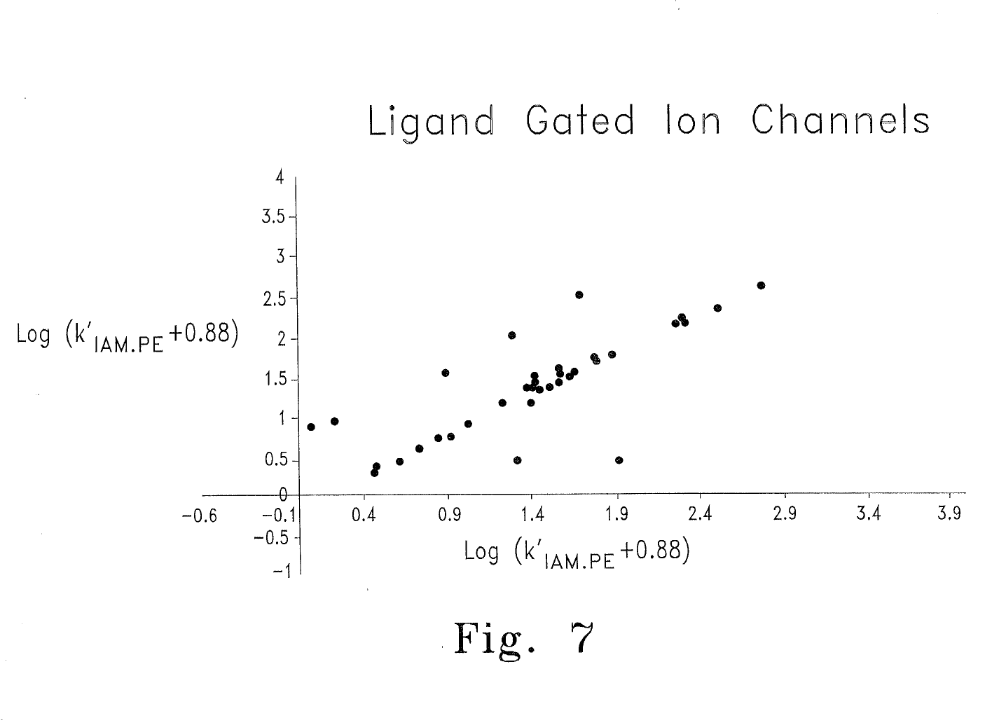

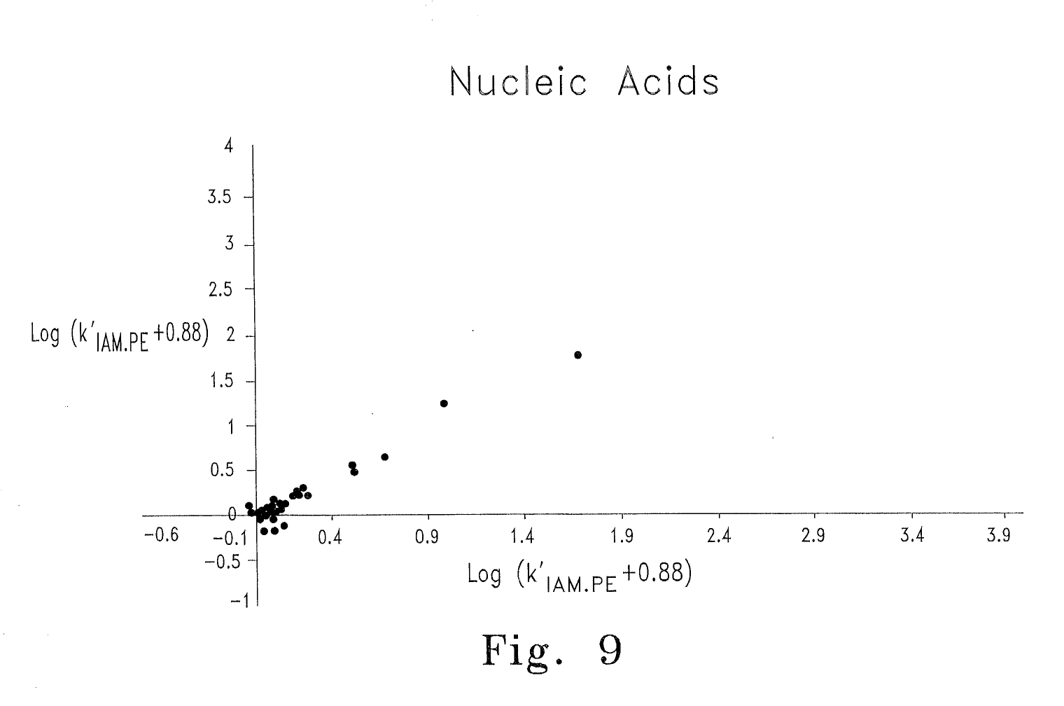

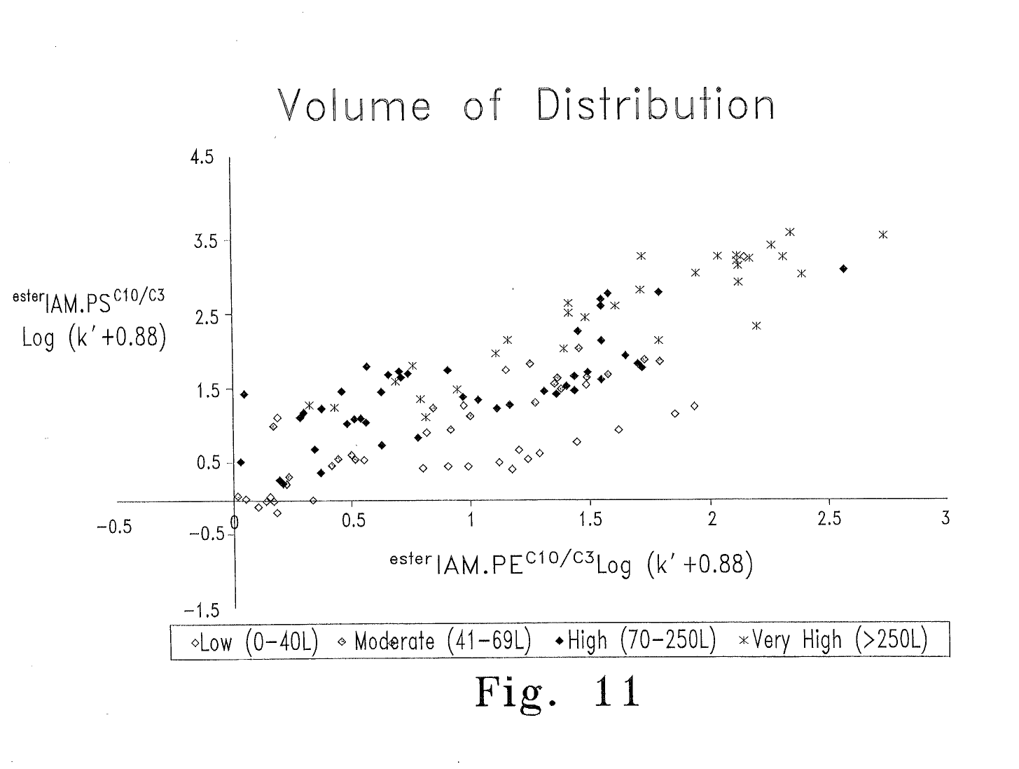

FIG. 1 is a graph presenting membrane affinity values forming at least a portion of a data set for use in drug discovery protocols in accordance with this invention. Each point on the graph represents the membrane affinity values for a known drug compound member of the data set used in accordance with this invention and represents the relative binding of said compound to a membrane mimetic surface comprising phosphatidyl ethanolamine (X-axis) and another membrane mimetic surface comprising phosphatidyl serine (Y-axis). It illustrates that all known drug substances exhibit a finite range of relative binding affinities for the respective membrane mimetic surfaces. FIGS. 2-10 illustrate relative membrane binding affinities for drugs interacting with specific classes of endogenous targets and show somewhat narrower ranges of relative binding affinities for drugs within each group. FIGS. 11-13 show the relationship between membrane binding affinities and levels of protein binding, volume of distribution and body clearance, respectively, and reveal patterns reflecting correlation between relative membrane binding affinities and those pharmacokinetic properties.

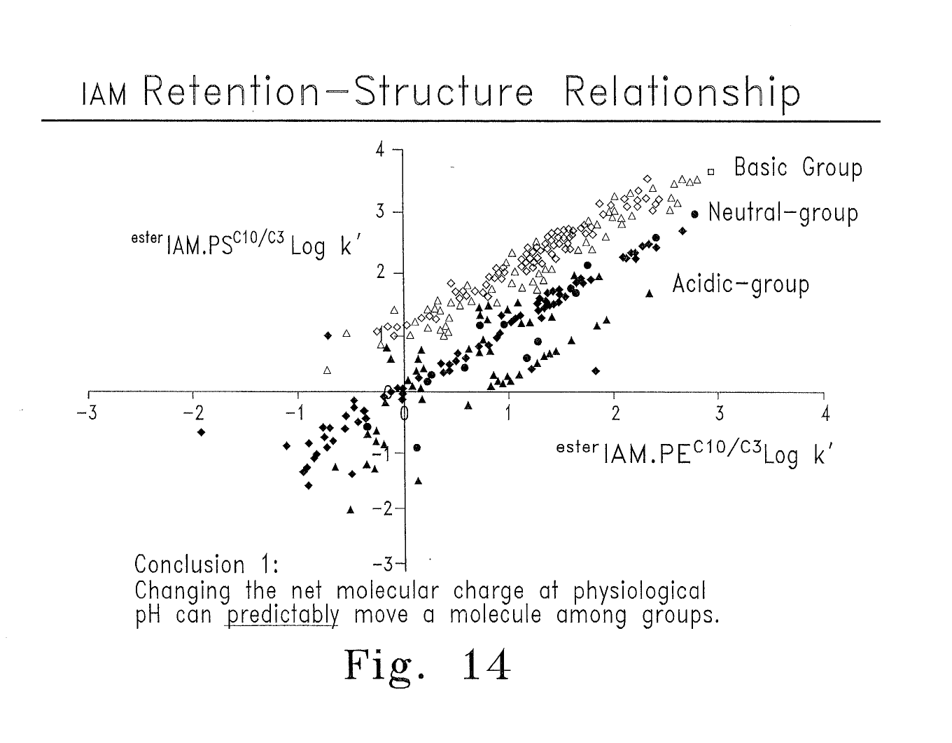

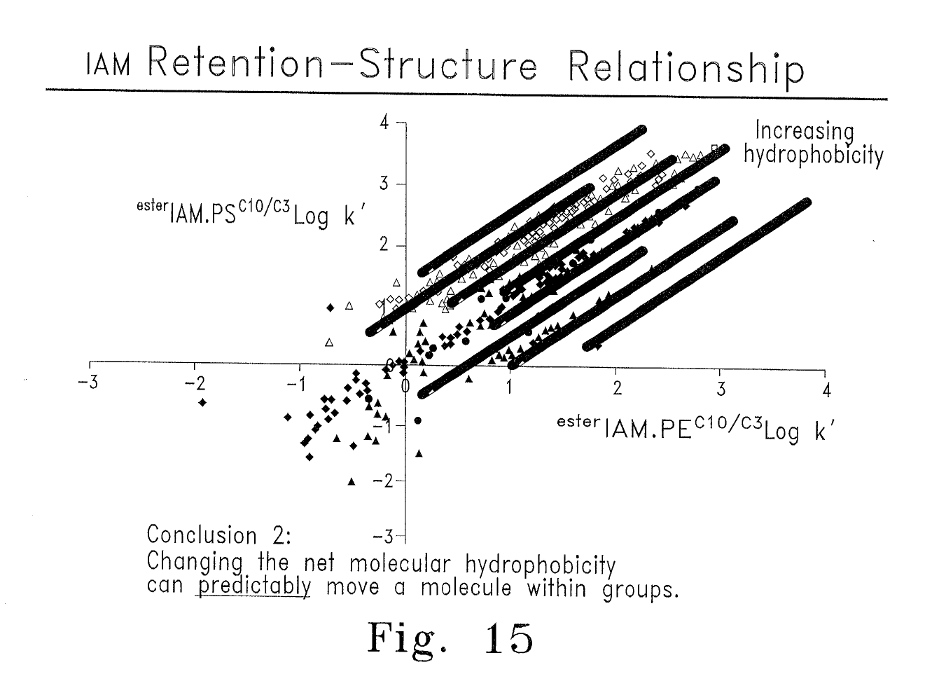

As illustrated in FIGS. 14 and 15, the chemical structure of the data set members can also be correlated with the respective values for affinity to membrane mimetic surfaces. Such correlations enable the prediction of changes in membrane affinity, and thus pharmacokinetic properties, effected by changes in chemical structure. Thus, the data set can provide basis for drug design to improve or modify pharmacokinetic properties. The data set, particularly when incorporated in a drug discovery system wherein the arrays of the data set are electronically stored in computer accessible format, can provide a powerful tool for the medicinal chemist seeking to design new drug substances with favorable pharmacokinetic properties.

Thus, in accordance with one embodiment of the invention there is provided a system for predicting pharmacokinetic properties of a proposed drug substance of known chemical structure based on correlation of the chemical structures of known drug substances, their known respective pharmacokinetic properties, and their empirically defined chemical structure/membrane affinity relationships. The system comprises a data storage device having in computer readable format a data set comprising chemical structures of a multiplicity of control compounds comprising drug substances and, for each compound, numeric values relating to the affinity of said compound to at least a negatively charged membrane mimetic surface and to a neutral membrane mimetic surface, and a numeric value relating to a known pharmacokinetic property, if any, of said compound. The system also includes a data entry device for entering the chemical structure or other search parameter in a computer readable format. The data entry device is in communication with a programmable microprocessor and the data storage device. The microprocessor is programmed to compare the chemical structure of the proposed drug substance entered into the data entry device with chemical structures in the data set to identify the chemical structures in the data set having a predetermined degree of similarity with the structure of the proposed drug substance. The microprocessor can also be programmed to compare, for example, membrane affinity values with those of the data set members to identify the data set members having membrane affinity values most similar to those of the proposed drug substance.

The system typically includes as well an output device in communication with the microprocessor capable of reporting, upon user request, the chemical structures or other identification of control compounds, if any, having the predetermined degree of similarity with the chemical structure of the proposed drug substance, or the membrane binding characteristics most similar to those of the proposed drug substance, and other data stored for said identified compound(s). Optionally the microprocessor can be programmed to report numeric values for the pharmacokinetic properties of the control compounds identified to meet the search criteria (structure similarity or membrane affinity similarity). Alternatively, the microprocessor can be programmed to identify other control compounds having membrane binding characteristics similar the membrane binding characteristics of the control compound(s) identified to have the predetermined degree of structural similarity or membrane binding similarity to that of the proposed drug substance. The identification of control compounds having similar membrane binding characteristics can be accomplished by calculating nearest neighbors to the point (Xₛ, Yₛ) in an array of points (X_{c}, Y_{c}) wherein Xₛ, Yₛ are the numeric values relating to the affinity of the control compound having the predetermined degree of structural similarity for a neutral membrane mimetic surface and a negatively charged membrane mimetic surface, respective, and X_{c} and Y_{c} are the numeric values relating to the affinity of the respective control compounds for a neutral membrane mimetic surface and a negatively charged membrane mimetic surface, respectively. The microprocessor can be programmed to report the numeric values for the pharmacokinetic properties of the control compounds having membrane binding characteristics similar to the membrane binding characteristics of the control compound(s) having the predetermined degree of chemical structure similarity.

In another embodiment the microprocessor is programmed to compare the membrane binding characteristics of the control compound(s) identified to have the predetermined degree of chemical structural similarity within empirically defined correlation between membrane binding characteristics and numeric values for each of several pharmacokinetic properties and to report the correlation predicted numeric values for each of the pharmacokinetic properties.

Another embodiment of the invention is a method for estimating the affinity of at test compound to membrane mimetic surface or a pharmacokinetic property of the test compound related to membrane affinity. The method comprises the steps of empirically defining a correlation between the structure of control compounds comprising drug substances and their respective affinities for membrane mimetic surfaces and preparing a data set comprising an array of control compound structures, values for their respective affinities for membrane mimetic surfaces, and values for their pharmacokinetic properties, if known, and conducting a search of the compound structures in the data set to identify control compounds having a structure with a predetermined degree of similarity with that of the test compound, and assigning the values for the membrane affinities and/or the pharmacokinetic properties of the identified control compound(s) as an estimate of the corresponding values and properties for the test compound.

The present invention also provides a system for predicting taxonomy of potential endogenous target macromolecules of a proposed drug substance of known chemical structure using a membrane affinity based correlation of the chemical structures of known drug substances, their endogenous target macromolecules, and their empirically defined chemical structure/membrane affinity relationships. The system comprises a data storage device having in computer readable format a data set comprising chemical structures of a multiplicity of control compounds comprising drug substances, and for each compound, numeric values relating to the affinity of said compounds to at least a negatively charged membrane mimetic surfaces and a neutral membrane mimetic surface, and a value relating to the identity or function of the known endogenous macromolecular target, if any, of said compound. It also includes a data entry device for entering the chemical structure or membrane binding data of the proposed drug substance in computer readable format and a programmable microprocessor in communication with said data entry device and said data storage device, said microprocessor programmed to compare the chemical structure of the proposed drug substance entered into the data entry device with chemical structures in the data set to identify the chemical structures in the data set having a predetermined degree of similarity with the structure of the proposed drug substance. Typically the system also includes an output device in communication with the microprocessor and capable of reporting, upon user request, the chemical structures or other identification of control compounds having the predetermined degree of similarity with the chemical structure of the proposed drug substance, and other data stored for said identified control compound(s).

The microprocessor can be programmed to report the numeric values for the pharmacokinetic properties of the control compound(s) identified to have the predetermined degree of structural similarity or to identify control compounds having membrane binding characteristics similar to the membrane binding characteristic of the control compound(s) identified to have the predetermined degree of structure similarity to that of the proposed drug substance. The identification of control compounds having similar membrane binding characteristics can be accomplished algorthmically by calculating nearest neighbors to the point (X_{S}, Y_{S}) in an array of points (X_{C}, Y_{C}) wherein X_{S}, Y_{S} are the numeric values relating to the affinity of a control compound having the predetermined degree of structural similarity for a neutral membrane mimetic surface and a negatively charged membrane mimetic surface, respectively, and X_{C}, Y_{C} are the numeric values relating to the affinity of the respective control compounds for a neutral membrane mimetic surface and a negatively charged membrane mimetic surface, respectively.

The microprocessor can be programmed to report the numeric values for the pharmacokinetic properties of the control compounds having membrane binding characteristics similar to the membrane binding characteristics of the control compound(s) having the predetermined degree of chemical structure similarity and/or to compare the membrane binding characteristics of the control compound(s) identified to have the predetermined degree of chemical structural similarity with an empirically defined correlation between membrane binding characteristics and numeric values for each of several pharmacokinetic properties and to report the correlation predicted numeric values for each of the pharmacokinetic properties.

Another aspect of the invention is a method for predicting pharmacokinetic properties for a proposed drug substance of known chemical structure. The method comprises the steps of selecting a database including, in computer readable format, chemical structures for a multiplicity of control compounds, said control compounds comprising known drug substances and for those known drug substances having known pharmacokinetic properties numeric values relating to at least a portion of said pharmacokinetic properties, searching the database for compounds having a chemical structure similar to that of the proposed drug substance and identifying those control compounds that have a predetermined degree of similarity to the proposed drug substance, identifying the pharmacokinetic properties of the control compounds having the predetermined degree of structural similarity, if any, to the proposed drug substance, and if no compounds are identified as having the predetermined degree of structural similarity, repeating the searching step using a lower predetermined degree of similarity until at least one control compound in the database is identified, and reporting the pharmacokinetic properties of the identified control compound(s) to predict the pharmacokinetic properties of the proposed drug substance.

In a related embodiment there is provided a method for predicting taxonomy of potential endogenous target macromolecules for a proposed drug substance of known chemical structure. The method comprises the steps of selecting a database including, in computer readable format, chemical structures for a multiplicity of control compounds, said control compounds comprising known drug substances each having a known endogenous target macromolecule, an for each known drug substance, a value corresponding to the taxonomy of its known endogenous target macromolecule, searching the database for compounds having a chemical structure similar to that of the proposed drug substance and identifying those control compounds that have a predetermined degree of similarity to the proposed drug substance, identifying the taxonomy of the endogenous target molecule of the control compounds having the predetermined degree of structural similarity, if any, to the proposed drug substance, and if no compounds are identified as having the predetermined degree of structural similarity, repeating the searching step using a lower predetermined degree of similarity until at least one compound in the database is identified, and using the taxonomy of the identified control compound(s) to predict the taxonomy of the target macromolecule of the proposed drug substance. Preferably the database further comprises in computer readable format, numeric values relating to the affinity of each control compound to at least a negatively charged membrane mimetic surface and/or numeric values relating to the affinity of each control compound to at least a negatively charged membrane mimetic surface and a neutral membrane mimetic surface.

The control compounds in the database can further comprise compounds not known to be drug substances but compounds for which is known and stored in the database numeric values relating to their respective relative affinities for at least a negatively charged membrane mimetic surface and a neutral membrane mimetic surface. It can occur in implementing the method that at least one of the control compounds identified to have the predetermined degree of structural similarity to the proposed drug substance is a compound for which the pharmacokinetic properties are not known. In that case the method further comprising the step of identifying the control compound or compounds in the database for which pharmacokinetic data is known and which has membrane binding properties most similar to the identified compound for which pharmacokinetic data is not known. In that regard the method can further comprise the step of displaying an array (X_{A}, Y_{B}) for each of at least a subset of the control compounds in the database, wherein X_{A} is the numeric value relating to the affinity of the control compound for one membrane mimetic surface and Y_{B} is the numeric value relating to the affinity of the control compound for a second membrane mimetic surface, where said subset of control compounds includes the compounds identified to have the predetermined degree of structural similarity to the proposed drug substance.

The present invention also enables a method for designing chemical structure modifications of a hit compound found to have target receptor binding activity in vitro to produce proposed chemical structures for test drug substances having improved pharmacokinetic properties relative to those of said hit compound. The method comprises the steps of selecting a database, including in computer readable format, structures for a multiplicity of control compounds, said compounds comprising known drug substances having predetermined pharmacokinetic properties, and for each compound numeric values relating to the predetermined pharmacokinetic properties, if known, and numeric values relating to the affinity of said compound to at least a neutral membrane mimetic surface and a negatively charged membrane mimetic surface, obtaining numeric values relating to the affinity of the hit compound to each of said membrane mimetic surfaces, correlating the respective known pharmacokinetic properties of the control compounds with their respective positions on a plot of an array of the points (X_{A}, Y_{B}) for at least a subset of the control compounds in the database wherein X_{A} is the numeric value relating to the affinity of the compound to one membrane mimetic surface and Y_{B} is the numeric value relating to the affinity of the compound to the other membrane mimetic surface, correlating the respective positions of the points (X_{A}, Y_{B}) in the array with chemical structural features of the respective control compounds represented by the respective points, identifying the position of the point (H_{A}, H_{B}) in the array of points (X_{A}, Y_{B}) wherein H_{A} is the numeric value relating to the relative affinity of the hit compound to one membrane mimetic surface and H_{B} is the numeric value relating to the relative affinity of the hit compound to the other membrane mimetic surface, identifying potential modifications of the chemical structure of the hit compound, with view of the respective correlations of chemical structure and array position and pharmacokinetic properties and array position to produce a predictable change in relative membrane binding affinities and pharmacokinetic values correlated therewith, and finally preparing the compound represented by the modified chemical structure and assaying same for target macromolecule binding activity, and, optionally, membrane binding affinities.

In another embodiment of this invention there is provided a method for estimating pharmacokinetic properties of a test drug substance. The method comprises the steps of identifying two or more membrane mimetic surfaces including a first membrane mimetic surface having a negatively charged surface and a second substantially neutral membrane mimetic surface, identifying a set of control compounds comprising drug substances having known pharmacokinetic properties and each pharmacokinetic property being quantified for each control compound by a numeric value within a range of numeric values relating to the pharmacokinetic property, defining for each control compound a numeric value related to its affinity for each membrane mimetic surface, defining for the test drug substance a numeric value related to its affinity for each membrane mimetic surface, for each pharmacokinetic property, identifying a subset of control compounds having similar pharmacokinetic property related numeric values and similar membrane affinity related values to establish a correlation between a subrange of pharmacokinetic property related values and a subrange of membrane affinity related values, comparing the membrane affinity related numeric values of the test drug substance with the correlated subranges of membrane affinity related values and pharmacokinetic property related values, and with respect to each pharmacokinetic property, identifying the subrange of numeric values related to that pharmacokinetic property correlated with the subrange of membrane affinity related numeric values for each membrane mimetic surface bracketing the respective membrane affinity related numeric values for the test drug substance, and selecting the subrange of pharmacokinetic property related numeric values for each pharmacokinetic property best matching the membrane affinity related numeric value for the test drug substance.

In a related aspect of the invention a method is provided for identifying the taxonomical classification and probable biological function of the endogenous target macromolecule of a test drug substance. The method comprises the steps of identifying two or more membrane mimetic surfaces including a first membrane mimetic surface having a negatively charged surface and a second substantially neutral membrane mimetic surface, identifying a set of control compounds comprising drug substances having known endogenous macromolecular targets, defining for each control compound a numeric value related to its affinity for each membrane mimetic surface, defining for the test drug substance a numeric value related to its affinity for each membrane mimetic surface, for each taxonomical target macromolecule classification, identifying a subset of control compounds having that same or similar target macromolecules and establishing a correlation between said taxonomical target macromolecule classification and membrane affinity values for the control compounds exhibiting said values, comparing the membrane affinity related numeric values for the test drug substance with the taxonomy correlated membrane affinity values for the control compounds, and selecting the taxonomical classification(s) that best match the membrane affinity values for the test drug substance.

Similarly a method is provided for estimating pharmacokinetic properties of a test drug substance. The method comprises the steps of identifying two or more membrane mimetic surfaces including a first membrane mimetic surface having a negatively charged surface and a second substantially neutral membrane mimetic surface, identifying a set of control compounds comprising drug substances having known pharmacokinetic properties and each pharmacokinetic property being quantified for each control compound by a numeric value within a range of numeric values relating to the pharmacokinetic property, defining for each control compound a numeric value related to its affinity for each membrane mimetic surface, defining for the test drug substance a numeric value related to its affinity for each membrane mimetic surface, and for each membrane mimetic surface or for a subset of the membrane mimetic surface, establishing, if possible, a mathematical correlation of membrane affinity related numeric values for the control compounds or a subset thereof with the numeric values relating to the respective pharmacokinetic properties using said correlation to calculate estimated numerical values for the respective pharmacokinetic properties for the test drug substance.

A similar method is enabled for identifying the taxonomical classification and probable biological function of the endogenous target macromolecule of a test drug substance. The method comprises the steps of identifying two or more membrane mimetic surfaces including a first membrane mimetic surface having a negatively charged surface and a second substantially neutral membrane mimetic surface, identifying a set of control compounds comprising drug substances having known endogenous macromolecular targets, defining for each control compound a numeric value related to its affinity for each membrane mimetic surface, defining for the test drug substance a numeric value related to its affinity for each membrane mimetic surface, comparing the membrane affinity related values for the test drug substance with those for the control compounds and identifying those control compounds having membrane affinity related values similar to those of the test compound, and identifying the taxonomical target macromolecule for those control compounds having membrane affinity values similar to those of the test drug substance.

In one other aspect of the invention there is provided a method for discovery of drug candidates having both potential for binding to a macromolecule recognized to be the endogenous target of a set of known drug substances and favorable pharmacokinetic properties, said method comprising the step of identifying chemical structural or substructural characteristics of said known drug substances, conducting a structure similarity search of a database comprising chemical structures of commercially available compounds to identify structures in the database of compounds having a threshold predetermined degree of structural similarity to at least one of the known drug substances, comparing the identified structures with those in a data set comprising the chemical structures of known drug substances and values corresponding to the relative affinity of said drug substances for at least two membrane mimetic surfaces and values corresponding to their pharmacokinetic properties, to eliminate, based on their probable lack of pharmacokinetic properties required for drug efficacy, at least a portion of those compounds identified to have the predetermined degree of structural similarity, and testing at least a subset of the compounds not eliminated by the correlated comparison for binding affinity to the macromolecular target.

The various embodiments of the invention are further illustrated in the following experimental examples.

General Experimental Procedures

A Bruker-Esquire LC/MS system (LC HP 1100 series) interfaced with an Gilson 235P autoinjector, and equipped with an orthogonal electrospray ionization (ESI) source and an ion trap mass analyzer was used for the collection of MS data. Single injection UV data collection was carried out on a HP 1100 series HPLC. All chromatographic data was collected with 15% acetonitrile in 0.01M PBS buffer (pH 7.4) as the mobile phase. Unless otherwise indicated, the mobile phase flow was programmed to follow a stepped gradient: constant flow rate of 0.5 mL/min for the first 10 minutes of data collection; the flow rate was then stepped to 4 mL/min over 20 minutes, after which time the mobile phase flow was held at a constant 4 mL/min. All IAM columns were subjected to a performance test prior to use, to ensure high quality and reproducibility in the chromatographic data. The column void volume (V₀) was also established for each column, as part of quality control.

All samples were obtained from commercial sources. All chemicals and solvents were of analytical grade and were used without further purification.

I. IAM columns

Membrane Affinity Fingerprints (MAFs) were determined on the following membrane mimetic surfaces: ^{ester}IAM.PC^{C10/C3}, ^{ester}IAM.PE^{C10/C3}, ^{ester}IAM.PS^{C10/C3}, IAM.SM^{C10/C3}, which were synthesized under strict QC according to known methods (see PCT/US98/17398 published as International Publication WO 99/10522, incorporated herein by reference). The stationary phase material (5 μm particle size, 80 Å pore size) was packed into 4.6×30 mm columns by Column Engineering, Ontario, Calif.

II. Phosphate buffer solution

The 0.01 M Phosphate Buffer Saline (PBS) solution was prepared by dissolving 0.2 g of potassium phosphate monobasic, 1.15 g of sodium phosphate dibasic, and 2.922 g of sodium chloride in 1 L of deionized water. The buffer was adjusted to pH=7.4 with an Accumet AP62 pH-meter, and filtered through a 0.2 μm nylon membrane (Millipore, type GN).

III. Sample handling and preparation

A stock solution of each compound was prepared in acetonitrile (or with an acetonitrile/methanol or DMSO mixture, depending on the solubility of the compound) at a concentration of 2 mg/mL. The samples were transferred onto a 96-well plate (one compound per well) on a TECAN Genesis RSP 150 robot, and diluted to 0.6 mg/mL by addition of 0.01 M PBS buffer. Typically, 30 μL of stock solution was pipetted into each well, and was diluted with 70 μL of PBS buffer. The resulting 96-well plate was used for the collection of flow injection data, and for generating the daughter plate to be used for the chromatographic data collection.

IV. Flow Injection

Mass spectral data was collected for each compound by flow injection. The resulting data constituted a reference database of mass spectral data, which was used as a reference file for deconvoluting chromatographic data produced during HPLC analysis of multi-compound mixtures (see below). In addition, the flow injection data allowed the identification of the compounds with low ionization efficiency, the chromatographic data of which needed to be generated by single injection UV detection.

The samples were delivered by autoinjection (injection volume 2 μL), and the UV data was collected at 220 nm, with 15% acetonitrile in 0.01 M PBS buffer eluting at 0.1 mL/min. The MS data was collected in positive and negative mode, with a low mass trap cutoff of 60 m/z, and a full scan range set from 100 to 700 m/z. The source parameters were configured as follows: Capillary −4000 V, Capillary exit −3500 V, Cap exit 80 V, skim 1 20 V, skim 2 6 V. Dry nitrogen gas (dry temperature 300° C.) was delivered at 10 L/min, and the nebulizer pressure was set to 30 psi. The data collected for each compound analyzed was saved into separate Bruker files.

The resulting Bruker-Esquire MS files were converted into a ChemStation format for data processing. The MS files were processed so as to generate for each compound its reference fragmentation peak list. The data processing included an average background subtraction and the exclusion of unwanted MS peaks (resulting from the mobile phase and other contaminants). In addition, the m/z peaks of relative intensity <3% of the parent ion were ignored. The peak list generated from each file/compound was saved into an integrated database (Examples of such integrated databases are SQL, ORACLE or other relational databases).

V. Compound Pooling

IAM chromatographic data was effected by analyzing mixtures of compounds (typically 20-50). The composition of the compound mixtures was determined by manually reading the reference compound flow injection data, and grouping compounds based non-overlapping m/z. Each compound mixture typically contained 20-50 compounds, at a total concentration of 0.6 mg/mL. The compounds with low ionization efficiency were each placed into individual wells for single injection analysis.

VI. Chromatographic Data Collection

The compound mixtures from section V were each transferred into the Gilson 235P autoinjector. The IAM columns were washed and equilibrated before and after each chromatographic run, and their performance and degradation was monitored from run to run. A column was discarded if it showed more than 5% degradation.

The samples were delivered by autoinjection (injection volume 20-30 μL, loading 12-18 μg of material), and the UV data was collected at 220 nm. The mobile phase (15% acetonitrile in 0.01 M PBS) was delivered at 0.5 mL/min for the first 10 min of data collection. The flow rate was then stepped from 0.5 mL/min to 4 mL/min over 20 minutes, after which time the flow rate was kept at 4 mL/min. The parameters for the MS instrument were identical as those used during flow injection data collection. The data collected for each chromatographic run was saved into a separate Bruker file.

The resulting Bruker-Esquire MS files were converted into a ChemStation format for data processing. The MS files were processed so as to detect, identify, and characterize the chromatographic peaks generated during each run. For each chromatographic file, the flow injection MS data of the compounds included in the chromatographic run was retrieved from the integrated database. The N m/z peaks (typically 2 or 3) of highest intensity were retained as the MS fingerprint of each compound and used as reference for peak identification in the HPLC chromatographic data obtained for the multicompound mixtures. Briefly, an algorithm is used to extract the chromatographic peaks from the profile data file, and match them with the MS fingerprint of a compound that was included in the run. A set of 2 (or 3) Single Ion Chromatograms (SIC) is returned for each compound, and each of the retention time, capacity factor (κ′), peak width, skewness, kurtosis, and other peak statistical parameters are calculated. To take into account the flow gradient profile used for the collection of the data, the retention volume and associated κ′ and peak statistical parameters were also calculated. Both the time and volume chromatographic information was stored in the integrated database.

Membrane Affinity Fingerprints (MAFs) and associated chromatographic parameters were determined for over 400 chemical entities with known pharmacological activities on up to four membrane mimetic surfaces (^{ester}IAM.PC^{C10/C3}, ^{ester}IAM.PE^{C10/C3}, ^{ester}IAM.PC^{C10/C3}, ^{ester}IAM.PC^{C10/C3}). The collection of this information is herein referred to as MAF database. Nearly 110 of these compounds have known ADME properties, and these were used to build an ADME prediction database/models for % protein binding, volume of distribution, and clearance.

VII. Data Analysis

The structure search routines were performed with CS ChemFinder 4.0, commercially available software capable of performing chemical structure/substructure similarity searches. Briefly, some of the compound information stored in the integrated database was retrieved (including, but not limited to, 2D structure, molecular weight, compound name) and stored in a file format searchable by ChemFinder. Substructure searches and/or complete structure similarity searches were conducted to identify compounds in the database that had structural similarity with the test compound, the ADME properties of which needed to be assessed. Other commercially available software for structure similarity searching can be substituted for ChemFinder for use in identifying compounds in the database having some predetermined degree of structural similarity with a test compound.

ChemFinder can perform exact structure/substructure searches and complete structure/substructure similarity searches. An exact search is based on atom connectivity comparison. The program compares the types of atoms and the order and way (bond type) in which they are connected, in the query and target molecules. If some atoms or bonds are missing, added, or different, the query structure and the target structure do not match. An exact search may be conducted with a complete compound structure or a substructure as query.

Similarity searches on the other hand rely instead on the notion of molecular descriptors. Each molecule, or portion of molecule (substructure), can be represented as a collection of molecular descriptors. ChemFinder uses a large number of descriptors. In the case of a complete structure similarity, the algorithm compares the number of descriptors the query and target molecules have in common to the number of descriptors they have in total. The ratio of these two values is called the Tanimoto coefficient, i.e., the similarity ratio. For substructure similarity searches, the concept is similar: the algorithm determines what percentage of descriptors in the query molecule are also present in the target. This value is the substructure similarity ratio.

ChemFinder structure searches were conducted in the MAF database using a compound of unknown ADME properties as query structure. The types of searches included exact substructure searches and structure similarity searches. For each test compound, the hits generated by the various ChemFinder structure searches were evaluated. A group of structures (typically 1-3), which showed the highest degree of similarity with the query compound were selected from the resulting structure search hit list. The structure selection was conducted according to structural and chemical criteria. These criteria included (but are not limited to) acid/base chemistry, number of heteroatoms, polarity, number of rings, lipophilicity, topology and size of the molecules.

The selected MAF database compounds were displayed on 4 graphs generated with MathSoft's S-plus 2000 graphics software. The graphs represented the Log κ′_{IAM PE} versus Log κ′_{IAM.PS} plots of 1) the entire MAF database, 2) the protein binding (% PB) standard curve, 3) the volume of distribution (Vd) standard curve, and 4) the clearance (CL) standard curve. The overlay of the compounds selected from the structure searches on the MAF database graph, gave insight into the MAF chemical space where the test compound might fall. The position (^{ester}IAM.PE^{C10/C3} and ^{ester}IAM.PS^{C10/C3} retention) of the test compound was estimated by comparison of its structure with that of the selected structure search hit compounds. The criteria used for the structure-retention correlation included (but are not limited to) acid/base chemistry, number of hydrogen bond donors, number of hydrogen bond acceptors, polarity, lipophilicity, ability to form intra- and intermolecular hydrogen bonds. Once the retention of the test compound was estimated, its predicted position was overlaid on the % PB, Vd, and CL standard curves, and its ADME properties were estimated by visually assessing the area where the compound was positioned.

Example of Membrane Affinity Based Array

The κ′ values described in the examples below characterize membrane binding constants of solutes measured as described above on immobilized artificial membrane (IAM) chromatography surfaces. For the purpose of the invention, membrane binding constant and κ′ are considered synonymous. Membrane binding constants are only one group of parameters that characterize membrane-solute interactions. Other parameters include the interfacial pKa, membrane enthalpy, the on-off kinetics of solutes from membrane surfaces, etc. Thus, although κ′ values are described in the examples, the invention is not limited to only parameters that characterize equilibrium binding between solutes and membranes.

Four κ′ values were experimentally obtained of approximately 400 commercial drug substances. (See Appendix.) A few non drug-substances were also evaluated, but most compounds in the database used to enable the present invention are commercial drugs. The IAM surfaces used for obtaining the κ′ values of the ˜400 compounds include: ^{ester}IAM.PC^{C10/C3}, ^{ester}IAM.PE^{C10/C3}, ^{ester}IAM.PS^{C10/C3}, IAM.SM^{C10/C3}.

A database containing four membrane binding parameters for ˜400 compounds is a 400×4 matrix. Because space is 3 dimensions, four parameters can not be plotted without a reduction in the number of variables. Principle component analysis (PCA) is an established method for viewing N-dimensional data when the data exceeds 3 parameters, i.e., when the number of parameters exceeds a 3 dimensional {x, y, z} coordinate system. Briefly, PCA calculates a new coordinate system that is mean centered to the data being analyzed. Most important, PCA plots of N-dimensional data show a coordinate system that maximizes the variance in the data. In other words, PCA is an established method for viewing the maximum separation of individual N-dimensional data points in 3 or less dimensions. In essence, PCA provides a new coordinate system, derived from the data itself, such that when the data is plotted in PCA coordinate space a maximum separation of the data is obtained. The dimensions (or axes) are sequentially denoted as principle component 1, principle component 2, and principle component 3. Complete details for preparing PCA plots are available.

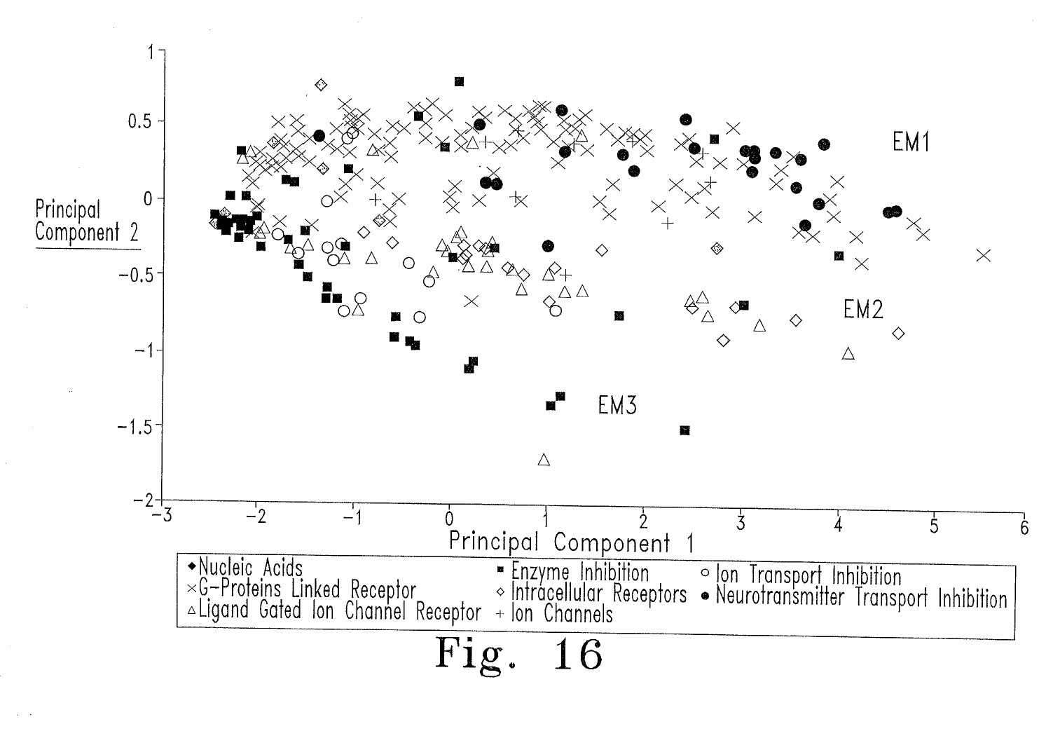

FIG. 16 is a 2-dimensional PCA plot of ˜400 commercial drugs using 4 membrane binding constants. The graph has three general regions labeled EM-1, EM-2 and EM-3 (EM denotes efficacy mechanisms). Although there are compounds with efficacy mechanisms in a region on the graph that differs from other compounds eliciting the same efficacy mechanisms, this does change the overall trends shown in FIG. 16. As shown in FIG. 16 (1) The EM-1 region has compounds that elicit therapeutic activity using G-coupled receptor proteins, ion channels, and neurotransmitter transport inhibition, (2) The EM-2 region contains compounds that act at intracellular receptors, ligand-gated ion channel receptors, and ion transport inhibitors, and (3) The EM-3 region has compounds that act predominantly as enzyme inhibitors. The three EM regions converge where nucleic acid type compounds reside on the graph. Thus, nucleic acids cannot be assigned to any particular region. In accordance with the present invention, FIG. 16 demonstrates that membrane-binding constants can be used to group compounds according to their efficacy mechanism.

An interesting aspect of FIG. 16 is the overlap among compounds with different efficacy mechanisms. For instance, the EM-1 curve includes both G-protein receptors and neurotransmitter transporters. This means that compounds can elicit different efficacy mechanisms yet have identical membrane binding properties. In other words, compounds with identical membrane binding constants may act at either G-protein receptors or neurotransmitter transporters. Virtually all classification schemes exhibit some level of overlap of the features being characterized and efficacy mechanism is thus no exception. The problem of overlapping features is exemplified in Table 1, which compares different features that have been used to classify drug discovery compounds; these include structure, therapeutic use, receptor, and efficacy mechanisms. With the exception of efficacy mechanisms, statistical methods to classify compounds into each of the other groups have previously been described in the art.

It is useful and routine in drug discovery to group compounds with similar features as described in Table 1. This allows drug discovery compounds to be sorted and pursued as drug leads according to favorable pharmacological features. Historically, there is always overlap among the features, probably because virtually all commercial drugs have side effects, which implies that they act at multiple sites. Consider the following 4 pharmacological features: structure, in vivo activity, receptor, and efficacy mechanism. It may be expected that compounds with similar structures have similar in vivo activity. However, as shown in Table 1, mescaline and amphetamine are both structural analogs of phenyethylamine, but mescaline causes hallucinations where amphetamines are stimulants, which are clearly different in vivo activities. Similarly, grouping compounds by therapeutic use also does not guarantee that the compounds within a group will have the same efficacy mechanism. For instance, chlorpromazine and scopolamine are both antiemetic drugs. Chlorpromazine acts at dopamine receptors whereas scopolamine acts upon muscarine receptors. Both are structurally different yet produce the same therapeutic result. These examples illustrate that there is no single method or process for grouping compounds that shows a unique relationship among structure, receptor, therapeutic use, and efficacy mechanism.

TABLE 1
Representative Methods to Classify Drugs.

| Drug | Receptor | Chemical Class | Therapeutic Class |
|---|---|---|---|
| 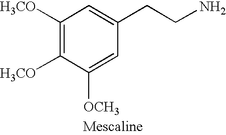 | Serotonin | phenyethylamine | hallucinogen |
| 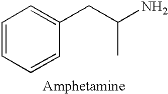 | multiple | phenethylamine | psychostimulant |
| 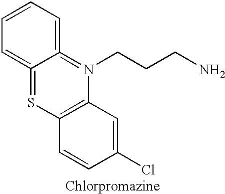 | dopamine | tricyclic | antiemetic |
| 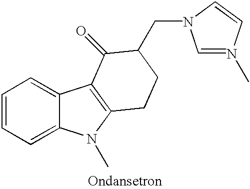 | serotonin | indole | antiemetic |
| 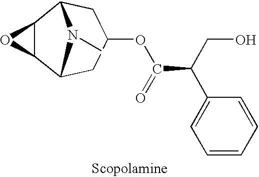 | muscarine | tropine | antiemetic |

The present invention shows that membrane-binding constants can be used to group compounds according to their efficacy mechanism. Equally important is that previous work has demonstrated that membrane-binding constants can group compounds according to their therapeutic use and receptor. Membrane binding is regulated by each compound's structure, as is the intrinsic pharmacological properties of compounds. Thus, the present invention supports the concept that all compound properties are regulated by compound structure, regardless of whether they have been measured or not. The correlation of membrane binding properties with all of the feature groups shown in Table 1 demonstrates the significance of the membrane binding properties of compounds in choosing which compounds to pursue in drug discovery.

Although overlapping features may frequently be undesirable in the classification of compounds, there are situations where characterizing overlap is particularly important in drug discovery. Overlapping features are thus not necessarily a disadvantage of classification methods.

Medicinal chemists search for compounds with commercially favorable pharmacological properties. Virtually always, the initial compound identified with activity does not elicit the complete pharmacological profile of a commercial drug and structural modifications are necessary. Thus, the most important attribute of any classification strategy is to be able to infer how to modify compounds to obtain desired activities. The PCA plot shown in FIG. 16 can not conveniently be used for the purpose of determining what structural modifications medicinal chemists may perform to obtain certain activities.

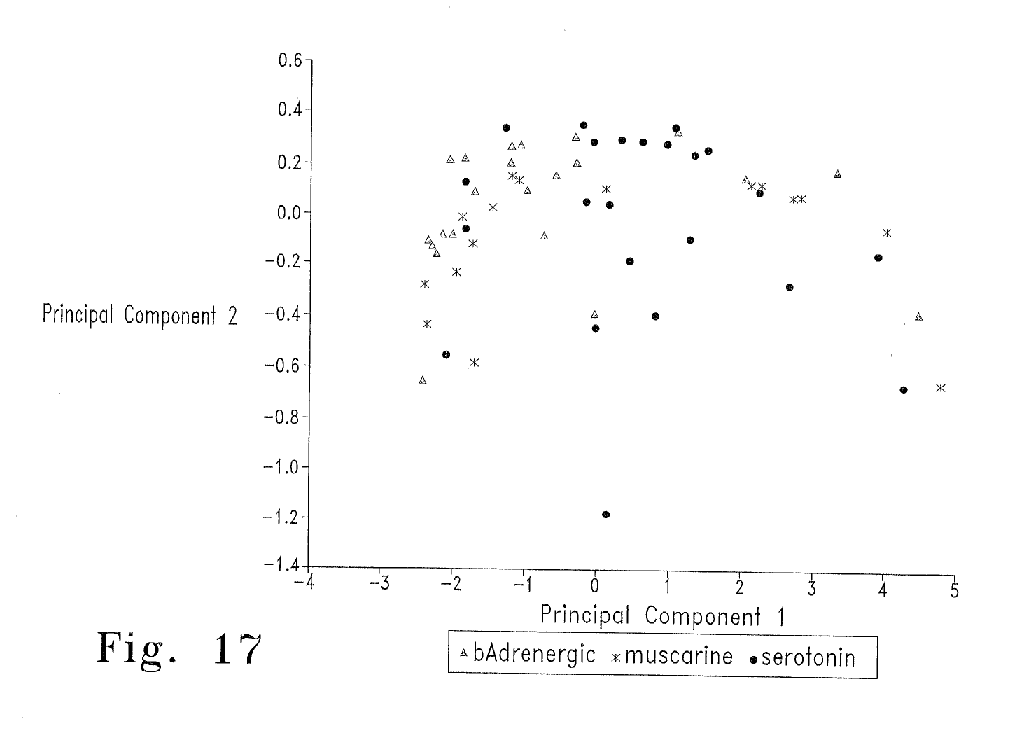

The PCA plot in FIG. 16 was obtained from using k′ values from four IAM columns: ^{ester}IAM.PC^{C10/C3}, ^{ester}IAM.PE^{C10/C3}, ^{ester}IAM.PS^{C10/C3}, IAM.SM^{C10/C3}. These IAM columns emulate the membrane lipid environment of cell membranes. Cell membranes contain proteins and lipids, and both of the cell membrane constituents can contain charges. Virtually all cells have negatively charged surfaces and the contribution of the surface charge density from proteins and lipids varies among cells. Some of the most abundant cellular lipids forming cell membranes include phosphatidylcholine, phosphatidylethanolamine, phosphatidylserine and sphingomyelin and the IAM columns used to obtain FIG. 16 were prepared from analogs of each of these membrane lipids. The PCA plot in FIG. 16 demonstrates a link membrane binding to several membrane lipids and efficacy mechanisms of commercial compounds. FIG. 17 is a PCA plot of four IAM κ′ values showing the intra-compound relationship within a receptor class.

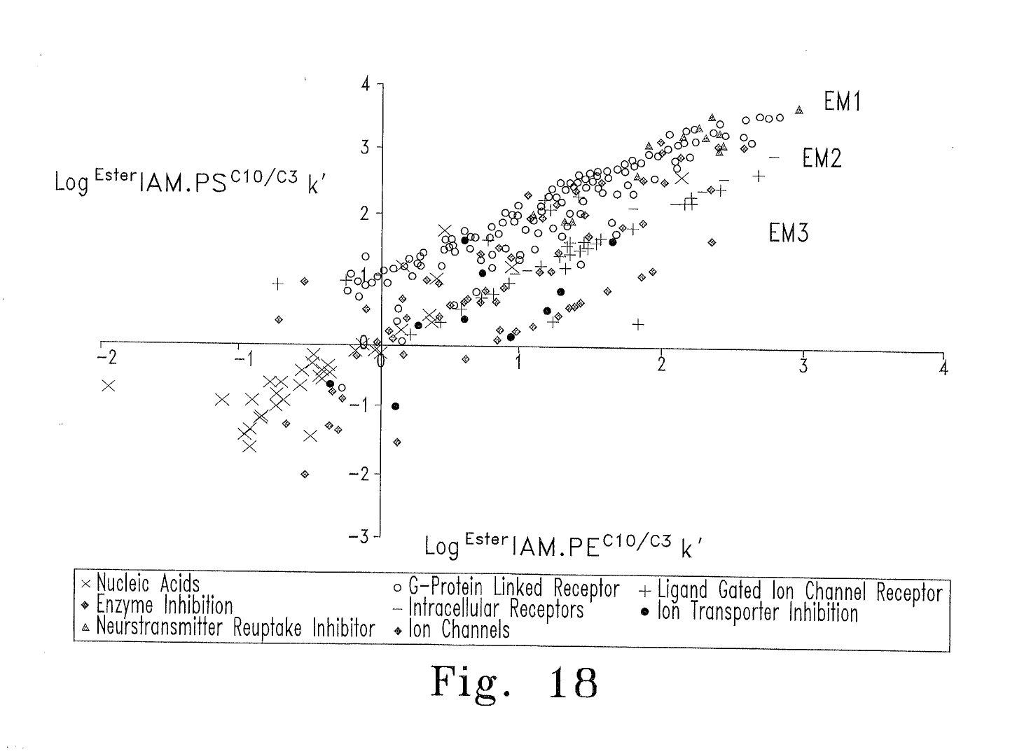

FIG. 18 is a plot of the efficacy mechanisms whereby actual membrane binding data is plotted for two IAM surfaces. The two IAM surfaces were ^{ester}IAM.PE^{C10/C3}, and ^{ester}IAM.PS^{C10/C3}. Similar to the PCA plot FIG. 16, FIG. 18 shows three EM regions. However, unlike the PCA plot, the three EM regions are approximately linear, albeit there are a few compounds that visually reside between the EM 1 and EM 2 lines.

The EM plot shown in FIG. 18 was obtained by plotting, on a logarithmic scale, the raw membrane binding data and consequently, the numerical values of the {x,y} coordinate system have physical significance. The physical significance is that the x coordinate provides the number of column volumes needed to elute the compound from an ^{ester}IAM.PE^{C10/C3} column, and the y coordinate provides the same information about the compound for an ^{ester}IAM.PS^{C10/C3} column. It is recognized that ^{ester}IAM.PS^{C10/C3} is a negatively charged chromatographic surface whereas ^{ester}IAM.PE^{C10/C3} is approximately a neutral surface. Thus, basic compounds will tend to reside near the EM-1 line, neutral compounds near the EM-2 line and acidic compounds near the EM-3 line shown in FIG. 16. The Lewis Acidic-base theory of molecules is useful in characterizing molecules that reside in different regions of the graph. In this regard, a few structures from each EM region are shown in the insert to FIG. 18 that shows the acid-base concept.

FIG. 18 allows the structure-dependent predictions of retention-times to be used to predict how to modify drug discovery compounds so that they reside in membrane space. Membrane space merely denotes where drug discovery compound should reside for eliciting the desired in vivo efficacy: the feature can be a therapeutic group, receptor, or EM in FIG. 16. Chromatographic retention mechanisms are well known in the art and numerous predictions of changes in retention times with structural changes have been described in the art. In the broadest sense the present invention utilizes 2 or more chromatography surfaces to obtain compound retention data that when plotted results in separation of at least a few groups among the bio-properties under study. The example used to demonstrate the present invention involves using the separated bio-properties in FIG. 16 and the chromatography retention data in FIG. 18. Clearly there is significant separation of bio-properties.

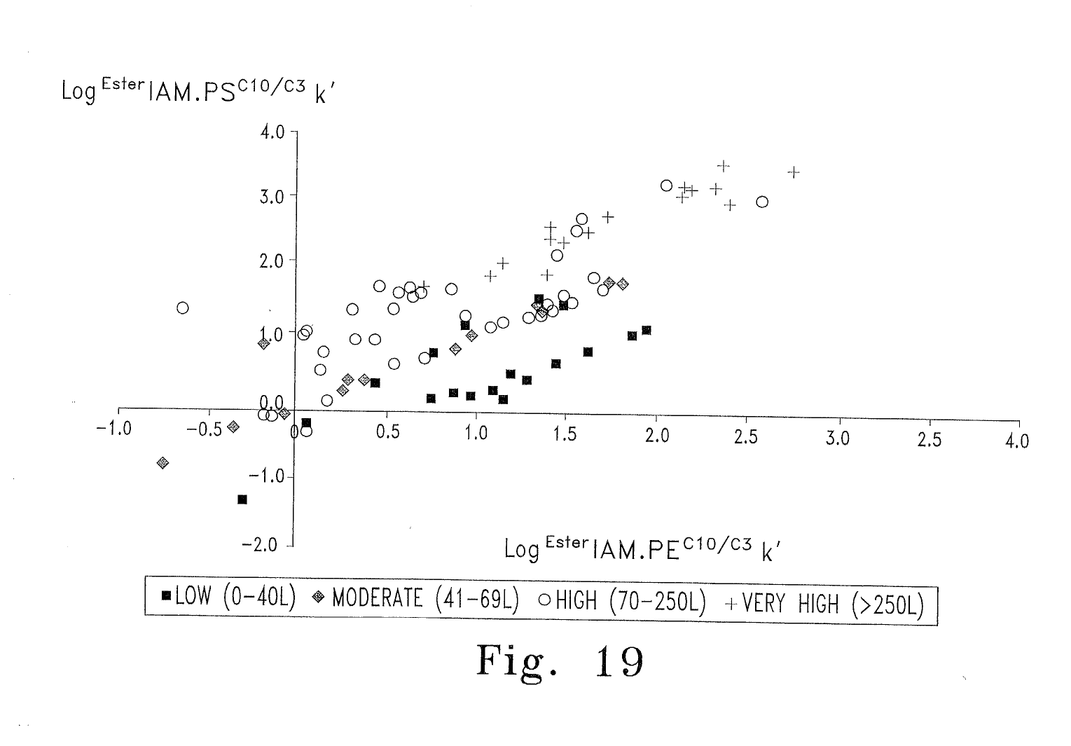

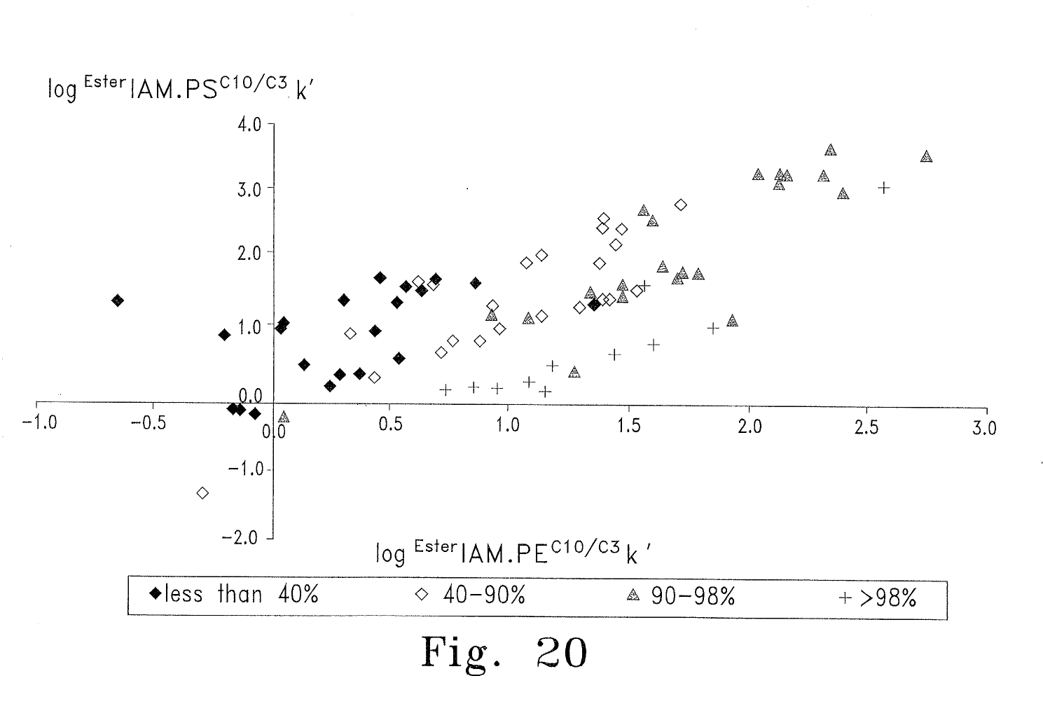

The bio-properties in FIG. 16 represent mechanistic and responsive pharmacodynamic properties of compounds. Similar relationships exists for the pharmacokinetic properties of commercial drugs and it was quite unexpected that the same two IAM surfaces used to forecast mechanistic and responsive pharmacodynamic properties shown in FIG. 18 can be used to model the pharmacokinetic properties of the same compounds. For instance FIGS. 19 and 20 respectively, show that volume of distribution and protein binding can be forecasted from chromatography retention time data obtained on IAM columns. The graphs shown in FIGS. 19 and 20 were used to predict the bio-properties described in each graph. Tables 2, 3, and 4 indicate that virtually all of the unknown compounds were correctly predicted.

TABLE 2
Examples of Vd predicted for six compounds using IAM k′ data.

| | Log | | | | |
|---|---|---|---|---|---|
| | PS | LOG PE | Predicted | Observed | Drug Name |
| 1 | 0.96 | 0.82 | HIGH (70-250 L) | 91 | Lamotrigine |
| 2 | 1.42 | 0.57 | VERY HIGH (>250 L) | 525 | Ropinirole |
| 3 | 3.37 | 2.33 | VERY HIGH (>250 L) | 1190 | Paroxetine |
| 4 | −0.90 | −1.17 | LOW (0-40 L) | 48.3 | Acyclovir |
| 5 | 2.54 | 1.87 | VERY HIGH (>250 L) | 217 | Diltiazem |
| 6 | 0.83 | 0.15 | HIGH (70-250 L) | 91 | Ranitidine |

TABLE 3
Examples of Protein Binding for Six Compounds Predicted Using Iam
K′ Data.

| | Log PS | LOG PE | Predicted | Observed | Drug Name |
|---|---|---|---|---|---|
| 1 | 0.96 | 0.82 | 40-90 | 55 | Lamotrigine |
| 2 | 1.42 | 0.57 | 40-90 | 68 | Ropinirole |
| 3 | 3.37 | 2.33 | Greater than 90 | 95 | Paroxetine |
| 4 | −0.90 | −1.17 | Less than 40 | 15 | Acyclovir |
| 5 | 2.54 | 1.87 | 40-90 | 78 | Diltiazem |
| 6 | 0.83 | 0.15 | Less than 40 | 15 | Ranitidine |

TABLE 4
Examples of total body clearance for six compounds being predicted
using IAM k′ data.

| | Log PS | LOG PE | Predicted | Observed | Drug Name |
|---|---|---|---|---|---|
| 1 | 0.96 | 0.82 | LOW < 0.327 | NA | Lamotrigine |
| 2 | 1.42 | 0.57 | HIGH > 0.327 | NA | Ropinirole |
| 3 | 3.37 | 2.33 | HIGH > 0.327 | 0.602 | Paroxetine |
| 4 | −0.90 | −1.17 | LOW < 0.327 | 0.235 | Acyclovir |
| 5 | 2.54 | 1.87 | HIGH > 0.327 | 0.84 | Diltiazem |
| 6 | 0.83 | 0.15 | HIGH > 0.327 | 0.728 | Ranitidine |

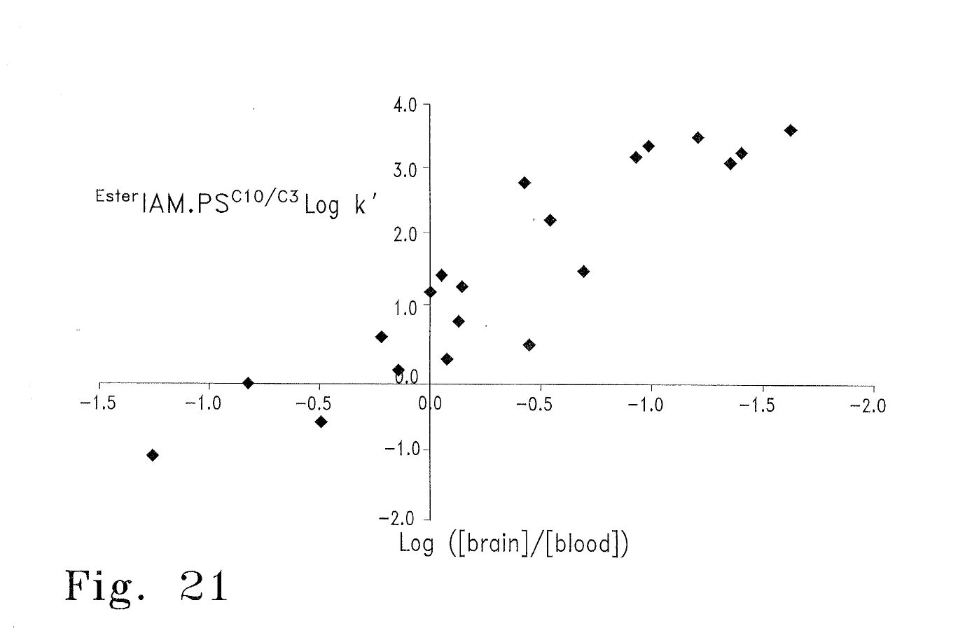

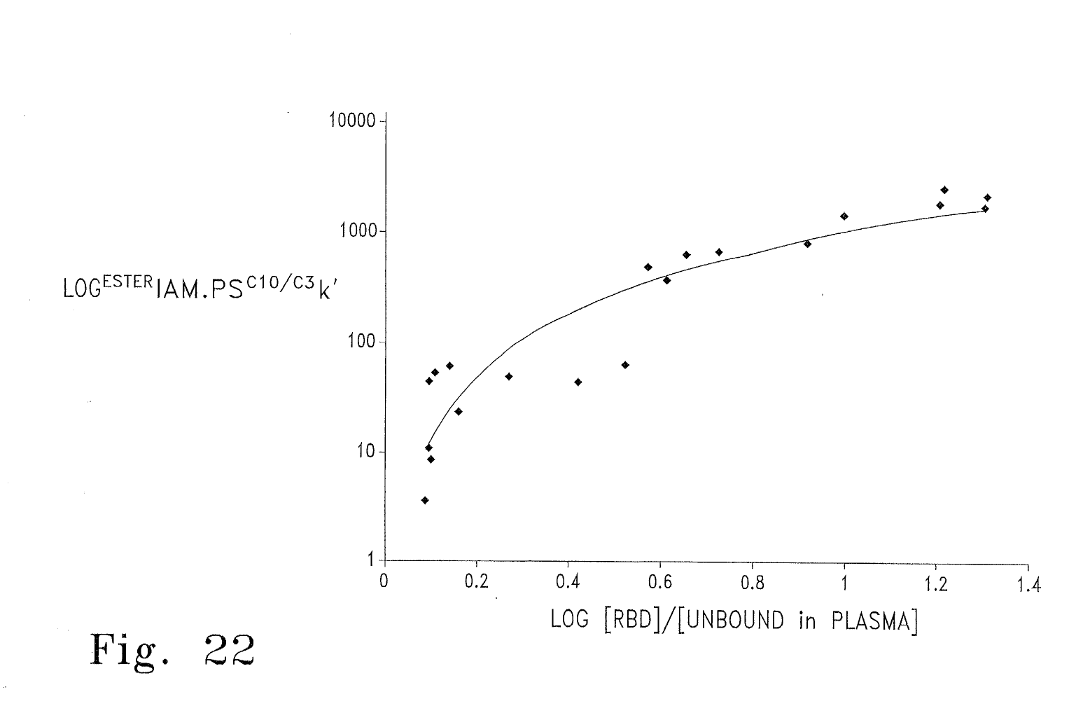

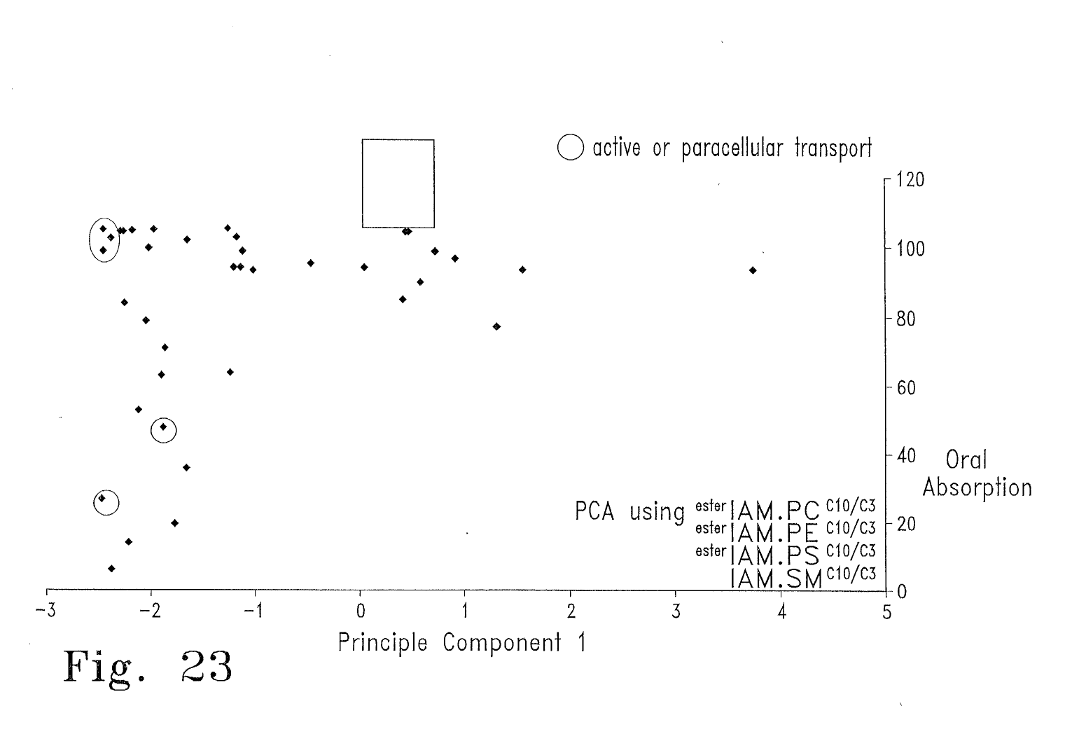

There is no restriction of the number of chromatography surfaces that can be used. The essential ingredients are to have (1) a set of bio-properties separated on a chromatography plot. Thus, FIGS. 21 and 22 shows that the equilibrium concentration of compounds in the CNS and peripheral blood/plasma are forecasted using only an ^{ester}IAM.PS^{C10/C3} column was used for this forecasting. An example of multiple columns is the forecasting of the oral absorption of compounds using 4 IAM surfaces as shown in FIG. 23. FIG. 23 is a PCA plot and is less intuitive in predicting the bio-properties but is potentially more useful because more than three dimensions can be evaluated. Nevertheless, FIGS. 19 through 23 clearly demonstrate that chromatographic systems can be used to forecast bio-properties, and in particular the information obtained from multiple chromatography surfaces have particular useful regions for predicting favorable properties of drug discovery compounds.

The present invention thus can be practiced, by predicting retention-times of drug discovery compounds. Chromatographic retention times depend on solute structure, the chromatographic surface, and the mobile phase. For discussion, only solute structural changes will be discussed, i.e., the mobile phase and chromatography columns are fixed. The two chromatography columns are ^{ester}IAM.PS^{C10/C3} (a negatively charged cationic surface) ^{ester}IAM.PE^{C10/C3} (a neutral surface). Fundamental chemistry is used to make predictions of the changes in retention times and the examples below are grouped according to the chemical principle used to predict changes in retention time.

For all examples described below, the experimental data was obtained on ^{ester}IAM.PE^{C10/C3} and ^{ester}IAM.PS^{C10/C3} columns and it is recognized that the only difference between these columns is the immobilized ligand. These columns have been prepared from the same silica, and packed under identical conditions. Thus they have the same particle size, porosity, void volume and other chromatographic parameters. The only difference between the columns is the ligand that was immobilized and therefore when comparing data obtained from ^{ster}IAM.PE^{C10/C3} columns to ^{ester}IAM.PS^{C10/C3} columns the chemist is comparing the chemical difference between the surfaces because the other column parameters are the same.

This provides physical significance to the κ′ values. For instance, trimethoprim has a κ′ of 10 on the ^{ester}IAM.PS^{C10/C3} column and 2 on the ^{ester}IAM.PE^{C10/C3} column. This indicates that (1) 10 column volumes of mobile phase are needed to elute [drug] from the ^{ester}IAM.PS^{C10/C3} column and only 2 column volumes from the ^{ester}IAM.PE^{C10/C3} column and (2) the compound has ˜5 times the affinity for the ^{ester}IAM.PS^{C10/C3} column compared to the other column. Thus, in the examples below a quantitative comparison between κ′ values obtain on different chromatography columns is possible. Furthermore, the only difference between the surfaces is the polar head group region on the surface. Both columns have approximately the same bonded phase thickness, volume of immobilized lipid, and hydrophobic region etc. The chemical difference between these surfaces resides in the first few angstroms of the surface where different polar headgroups resides; underneath the polar headgroup resides the same hydrophobic thickness and content.

Several chemical mechanisms or factors determine the retention of solutes on IAM surfaces. Solute binding to IAM surfaces is determined not only by acid-base chemistry but also the solute's hydrophobicity. It is common to compare solute binding on new chromatographic surfaces to the binding on hydrophobic chromatography surfaces prepared from immobilizing linear alky chains (e.g., C6, C8, C10, C18 chromatography columns). Hydrophobic columns obviously retain compounds predominantly by a hydrophobic mechanism and therefore a hydrophobic index for a new column can be defined. In this regard, neutral ^{ester}IAM.PC^{C10/C3} surface retains compounds similar to C6-C8 columns. The key point is that in addition to acid-base mechanisms, hydrophobicity is a significant factor determining the retention of compounds on IAM surfaces.

### EXAMPLE 1

### The Chemical Principle: Opposite Charges Attract

The first example uses the established chemical principle that opposite charges between two molecules or a molecule and a surface result in attraction between the two chemical systems. Briefly, cations exhibit different levels of affinity for different anions.

Under the pH 7.1 mobile phase conditions used to measure membrane binding data described in the present invention, the ^{ester}IAM.PE^{C10/C3} surface is neutral. However, at this mobile phase pH the ^{ester}IAM.PS^{C10/C3} surface has two negative charges and one positive charge; the net charge on the surface is negative and therefore ^{ester}IAM.PS^{C10/C3} is an anionic surface. The EM-1 region reflects the chemical fact that basic compounds are expected to have higher affinity for negatively charged surfaces compared to neutral surface. A basic compound may be either neutral or cationic when protonated. Consider the mobile phase condition whereby the mobile phase pKa is well below the pKa of the compound and therefore the compound is virtually completely ionized, i.e. the compound bears a full positive charge. Further, consider a set of compounds with one full positive charge. The set of cations may have the same molecular charge, however, the polar surface area, molecular surface area, molecular volume, hydrophobicity etc. will vary among the cation set. Chromatographic generalizations that can be attributed to the set of cations include the following:

1. All compounds in the set will have higher affinity for the ^{ester}IAM.PS^{C10/C3} surface because of the cation charge each molecule.

2. Proportional increase in hydrophobicity will have a proportional increase in retention time on both columns.

3. Proportional increases in the fractional polar surface area will have a proportional decrease in retention on both surfaces.

4. Proportional increase in the numbers of hydrogen bond donors or acceptors will decrease retention on both surfaces.

5. Compounds with phenolic groups will have reduced affinity for both columns relative to those without phenolic groups.

6. Compounds with aromatic ether groups will have decreased affinity for both columns relative to those without aromatic ether groups.

7. Compounds with both acid and basic functionality (ampholites) will have decreased affinity for ^{ester}IAM.PS^{C10/C3} relative to compounds that are only cationic.

Collectively, one would expect the set of cation κ′ values for both IAM columns,

{^{cpd1}κ′_{PE}, ^{cpd1}κ′_{PS}}, {^{cpd2}κ′_{PE}, ^{cpd2}κ′_{PS}}, . . . ,{^{cpd-n}κ′_{PE}, ^{cpd-n}κ′_{PS}}}

to have a linear relationship, which intercepts the y-axes above zero and has a positive slope. This is the EM-1 curve (FIG. 18). Thus, the intercept of the EM-1 line represent higher affinity of cations for negatively charged surfaces, and the positive slope of the EM-1 line shows the direction of increasing compound hydrophobicity and decreasing fractional polar surface area. As indicated above, these generalizations reside from chemical intuition of the scientist and provide general guidelines for predicting the change in elution of compounds for changes in molecular structure.

### EXAMPLE 2

### The Chemical Principle: Similar Charges Repel

The second example uses the established chemical principle that similar charges between two molecules or a molecule and a surface result in repulsion between the two chemical systems. Briefly, anions exhibit different levels of repulsion for different anions.

A monoprotic acidic compound may be either neutral or anionic depending on the solvent or mobile phase. Consider the mobile phase condition whereby the mobile phase pKa is well above the pKa of the compound and therefore the compound is virtually completely ionized, i.e. the compound bears a full negative charge. Further, consider a set of compounds with one fall negative charge. The set of anions may have the same molecular charge, however, the polar surface area, molecular surface area, molecular volume, hydrophobicity etc. will vary among the anion set. Chromatographic generalizations that can be attributed to the set of anions include the following:

1. All compounds in the set will have lower affinity for the ^{ester}IAM.PS^{C10/C3} surface because of the anion-anion repulsion between the chemical systems

2. A proportional increase in hydrophobicity will have a proportional increase in retention time on both columns.

3. A proportional increase in the fractional polar surface area will have a proportional decrease in retention on both surfaces.

4. Increasing the numbers of hydrogen bond donors or acceptors will decrease retention on both surfaces.

5. Compounds with phenolic groups will have reduced affinity for both columns relative to those without phenolic groups.

6. Compounds with aromatic ether groups will have decreased affinity for both columns relative to those without aromatic ether groups.

Collectively, one would expect the set of anion κ′ values

{{^{cpd1}κ′_{PE}, ^{cpd1}κ′_{PS}}, {^{cpd2}κ′_{PE}, ^{cpd2}κ′_{PS}}, . . . {^{cpd-n}κ′_{PE}, ^{cpd-n}κ′_{PS}}}

to have a linear relationship, which intercepts the y-axes below zero and have a positive slope. This is the EM-3 curve. Thus, the intercept of the EM-3 line represent lower affinity of anions for negatively charged surfaces (e.g., ^{ester}IAM.PS^{C10/C3} surfaces), and the positive slope of the EM-3 line shows the direction of increasing compound hydrophobicity, decreasing fractional polar surface area, etc.

### EXAMPLE 3

### The Chemical Principle: Neutral Compounds Are Not Attracted or Repelled from Negative or Positive Charged Surfaces

The third example uses the established chemical principle that neutral compounds are not attracted or repelled from another chemical system that has a charge.

Neutral compounds are any compounds whose total ionic charge is zero. This includes all compounds that lack ionizing groups and those which have equal number of acidic and basic ionizing group (ampholytes). Compounds which are basic or acidic but are not ionized at pH=7.1 are included, as well.

Such compounds lack ionic interactions with the IAM surface so should lack repulsion or attractive mechanisms associated with their membrane interactions. As such, a set of neutral compounds should have the following attributes:

1. All compounds in the set will have little affinity difference for the ^{ester}IAM.PS^{C10/C3} surface over ^{ester}IAM.PE^{C10/C3} or other lipid surfaces.

2. Compounds with a proportional increase in hydrophobicity will have a proportional increase in retention time on both columns.

3. Compounds with a proportional increase in the fractional polar surface area will have a proportional decrease in retention on both surfaces.

4. Compounds with increased numbers of hydrogen bond donors or acceptors will have a decreased retention on both surfaces.

5. Compounds with chemically fused rings will have increased retention on all surfaces over compounds that have equal numbers of rings systems but not fused linearly.

Collectively, one would expect the set of neutral κ′ values

{{^{cpd1}κ′_{PE}, ^{cpd1}κ′_{PS}}, {^{cpd2}κ′_{PE}, ^{cpd2}κ′_{PS}}, . . . {^{cpd-n}κ′_{PE}, ^{cpd-n}κ′_{PS}}}

to form a linear relationship, which intercepts the y-axes at approximately, zero and have a positive slope. This is the EM-2 curve in FIG. 18.

### EXAMPLE 4

### Hydrophobicity

Hydrophobicity is a function of a compound's preference to partition into a hydrophobic environment over a hydrophilic environment. IAM surfaces are excellent models of a hydrophobic environment. Chromatographic generalizations are then possible such that:

1. All compounds will have increased affinity on IAM surfaces as a proportional increase in hydrophobicity is made.

2. Increasing hydrophobicity by increasing the number of fused aromatic ring systems will increase affinity for all IAM-type columns versus non-fused systems.

### EXAMPLE 5

### Polar Surface Area

Polar surface area is the amount of chemical space occupied on the surface of a molecule that is considered to be non-hydrophobic. Electronic composition and three dimensional structure are variables which effect polar surface area. Various mathematical approaches are used to determine polar surface area. Chemically, ionizing groups will greatly increase polar surface area, as will heteroatoms. Hydrocarbons decrease polar surface area.

Chromatographic generalizations based on polar surface area are readily made for IAM columns, such that increasing polar surface area on a molecule leads to a proportional decrease of affinity for IAM columns. This phenomenon should hold true for all surfaces and molecule types.

### EXAMPLE 6

### Bulk pKa Regulates Retention

Another retention factor on IAM columns involves the pKa of the compound. As discussed, the EM-1 group in FIG. 18 is predominantly comprised of basic compounds. Consider two compounds with the same amount of molecular hydrophobicity; the compound with the higher bulk pKa will have a larger fraction ionized in the mobile phase (i.e., more positively charged solute molecules) and therefore have higher affinity for the ^{ester}IAM.PS^{C10/C3} surface.

The present invention allows medicinal chemists to use chemical intuition to design, or modify molecules, so that they reside in different EM regions, therapeutic groups, or receptor groups. The general guidelines for the chemical intuition are to preparing/modifying compounds such that the compounds elution on 2 or more chromatography columns is consistent with compound elution of training set compounds. Training set compounds are compounds with a desired bio-property. It is expected, than typically basic compounds will reside on the EM-1 curve, neutral compounds on the EM-2 curve and (3) acidic compounds on the EM-3. It is well recognized that these general principles may not always true because of many of the subtle chemical mechanisms controlling the binding of solutes to membranes. Nevertheless, this is a clear starting point for medicinal chemists to use acid/base chemistry (1) predict the membrane binding properties that are needed for moving a compound within or between EM groups, and (2) experimentally determining if the structural modification(s) resulted in the desired change in membrane binding properties by measuring the retention times of the modified compound on the IAM surface.

One principle of the invention is that the retention mechanisms associated with IAM columns can be used as basis for modification of molecules to occupy predetermined regions of membrane space and therefore EM space, receptor space, therapeutic use space, etc. FIGS. 14 and 15 summarize the general principles of the present invention regarding the design of compounds for the purpose of drug discovery.

### EXAMPLE 7

### In Silico ADME Predictions

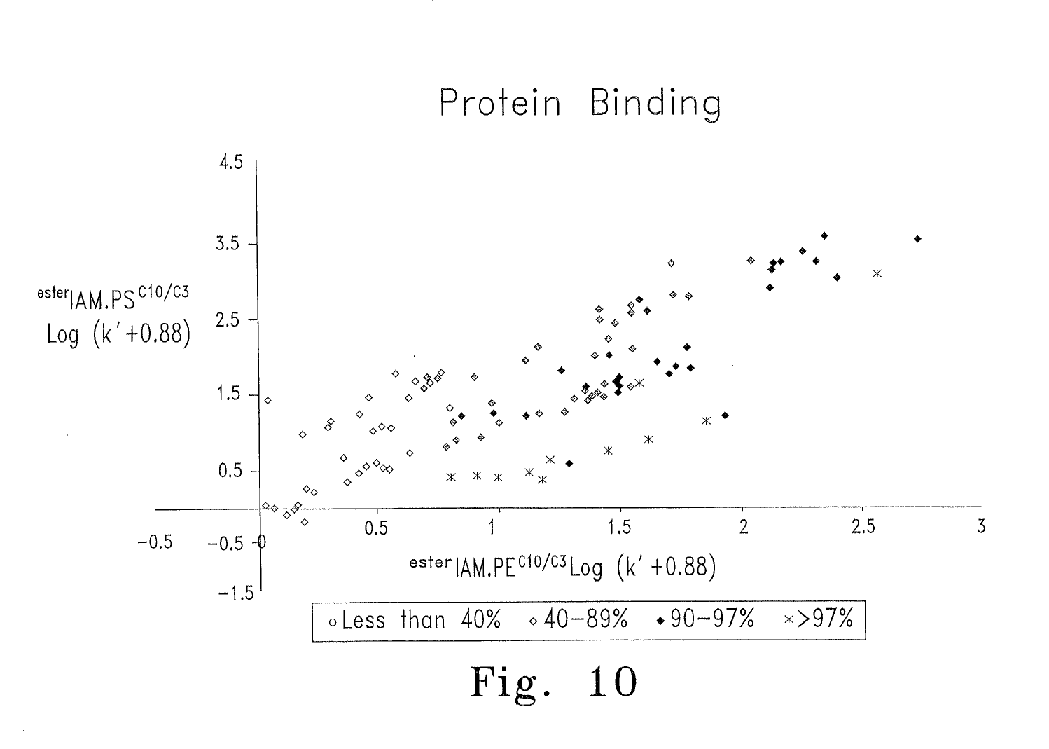

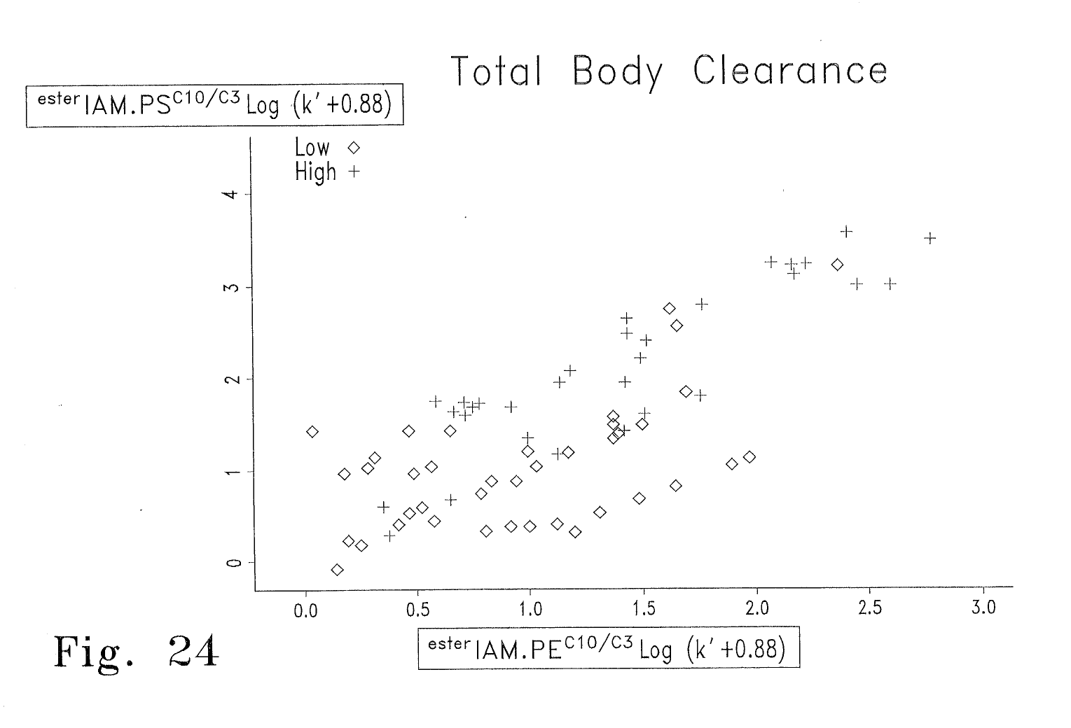

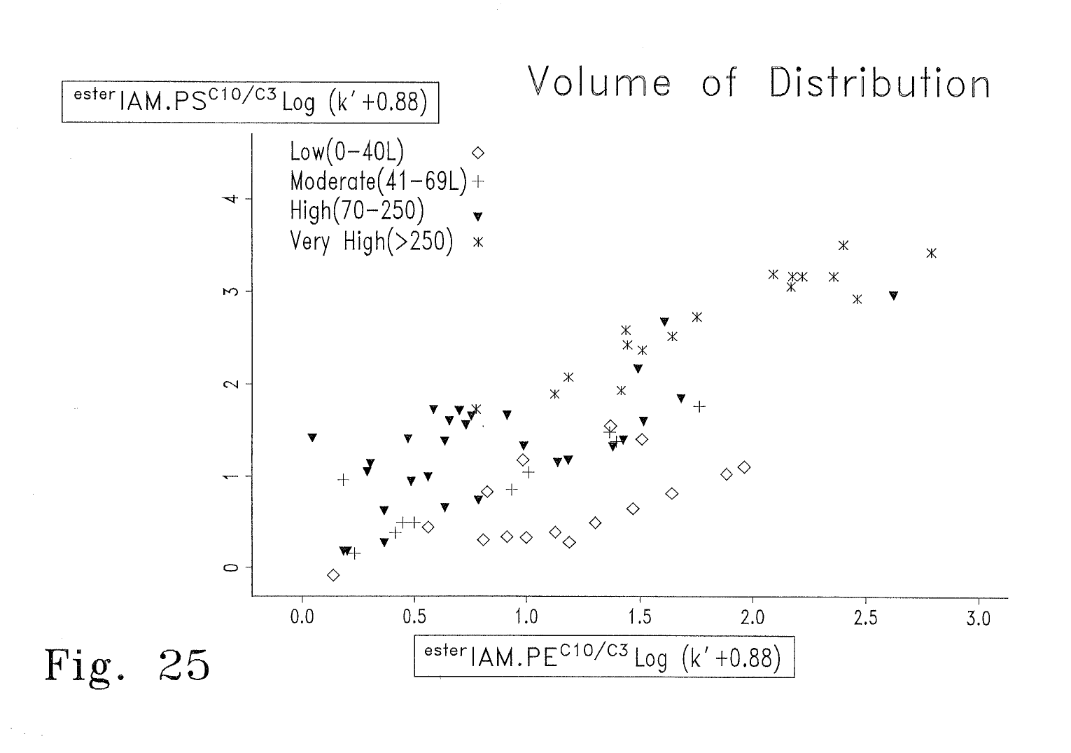

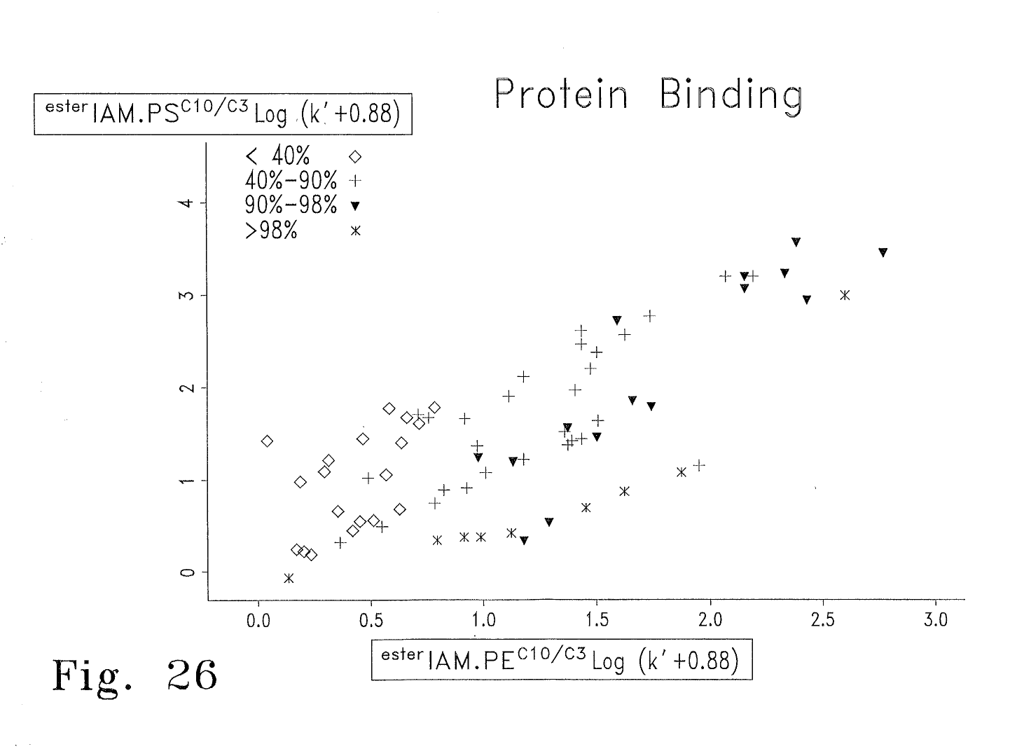

The objective of the experiment was to use the standard curves generated for volume of distribution, protein binding and clearance as a reference for in silico predictions of ADME. A non conventional approach to in silico predictions was used. A conventional approach would choose parameters (calculated or experimental) to try to calculate the membrane binding properties. In contrast to this approach, a simple topological search method was combined with a set of standard curves that were generated using compounds with known ADME data. FIGS. 24-26 are the standard curves that were generated with ˜80 known drugs. Each data point on the graphs is a drug with known ADME properties. The standard curves shown in FIGS. 24-26 were used for the in silico ADME predictions in Study 1. Later on, the standard curves for protein binding, volume of distribution, and clearance were completed to include ˜100 drugs with known ADME properties (FIGS. 10, 11 and 12). These curves were used in Study 2.

Two independent studies were carried out to test the aforementioned non-conventional in silico prediction method, and evaluate its scope of use.

Study 1

A group of test compounds was generated from a similarity search based on a parent compound (Clonazepam). Clonazepam belongs to a group of compounds (benzodiazepines) that acts at the GABA receptor. The benzodiazepine group comprises several structurally related drugs. The purpose of selecting a member of the Benzodiazepine family as the parent compound was to test the method with a pool of compounds with high structural similarity, as a first study test. The objective was then to expand the study to chemically diverse compounds to test the limitations of the method, and its scope of use (Study 2).

The Membrane Affinity Fingerprint (MAF) on two IAM surfaces (^{ester}IAM.PE^{C10/C3} and ^{ester}IAM.PS^{C10/C3}) and ADME properties of Clonazepam were known. The objective was to estimate the MAFs of the structurally similar compounds based on their structural and chemical features, and predict their ADME properties using the three aforementioned standard curves (Vd, CL, % protein binding). The ADME prediction consisted of assigning each hit compound to the correct category for each ADME property:

| Clearance (CL): | Low | | High | |
|---|---|---|---|---|
| | (<0.378 L/min) | | (>0.378 L/min) | |
| Volume of Distribution (Vd): | 0-40 L | 41-69 L | 70-250 L | >250 L |
| Protein binding: | <40% | 40-89% | 90-97% | >97% |

The similarity search was conducted with ChemFinder on a library of about 80 chemically diverse compounds. ChemFinder uses the Tanimoto equation to determine if compounds are similar. You can specify the desired degree of similarity in the Searching tab of the Preferences dialog of the program. The search relies on the notion of molecular descriptors. Each compound can be represented by a collection of qualitative terms (descriptors) that describe general aspects of the structure. The Tanimoto similarity test is commutative: it compares the number of descriptors chemical structures have in common to the number of descriptors they have in total. The ratio of these two values is the similarity ratio.

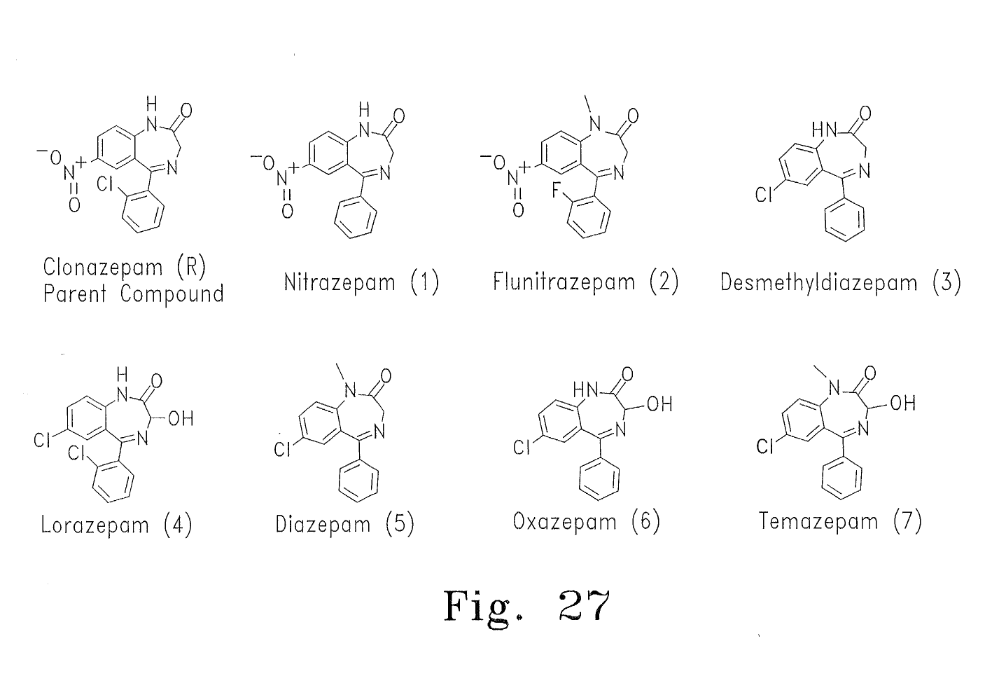

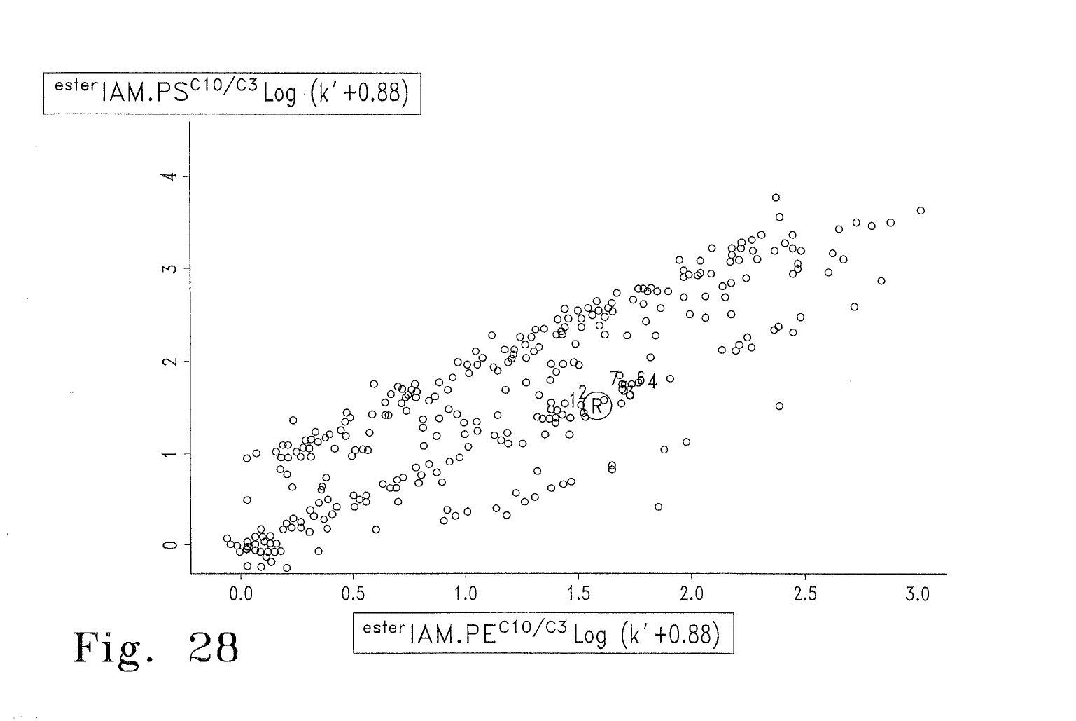

The ChemFinder search was set at 80% similarity, and identified 7 highly structurally similar hit compounds, all of which belonging to the Benzodiazepine family, as expected: Nitrazepam (1), Flunitrazepam (2), Desmethyldiazepam (3), Lorazepam (4), Diazepam (5), Oxazepam (6), and Temazepam (7). The chemical structures of the compounds are shown on FIG. 27.

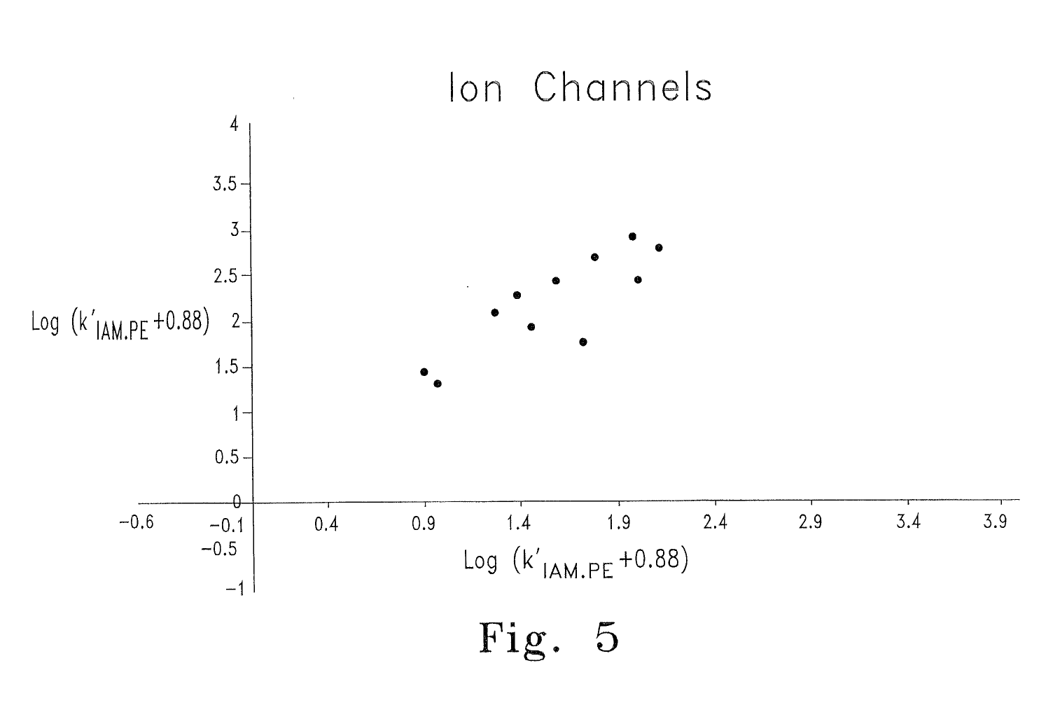

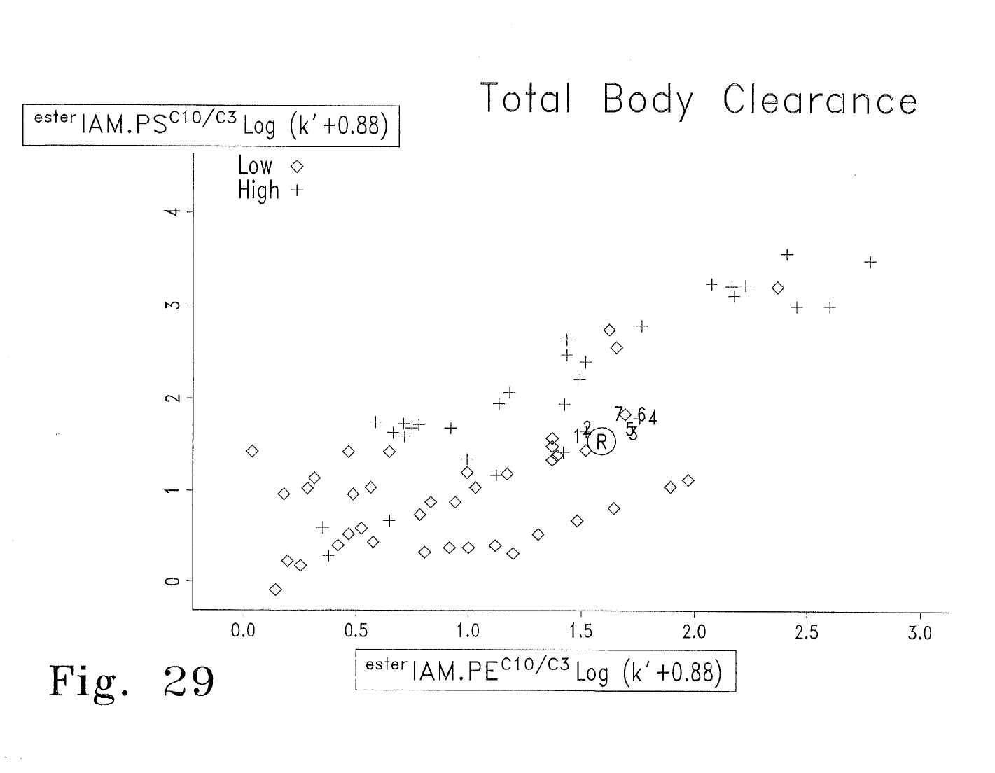

The coordinates of the reference compound Clonazepam on the general graph were known (see FIG. 28, data point labeled “R”): its capacity factors (κ′) on ^{ester}IAM.PE^{C10/C3} and ^{ester}IAM.PS^{C10/C3} had been determined previously. Each data point on the graph shown on FIG. 5 represents a drugs with a known MAF on ^{ester}IAM.PE^{C10/C3} and ^{ester}IAM.PS^{C10/C3}. This graph represents the chemical space covered by approximately 400 drugs. The ADME properties of 80 of these 400 drugs were known. These 80 compounds were used to generate the standard curves for volume of distribution, protein binding, and clearance. The ADME properties of Clonazepam were also known: CL=0.109 L/min, Vd=224 L, and Protein binding=86%. Each of the 7 hit compounds was positioned on the general graph, relative to Clonazepam and to one another, according to structural and chemical criteria.

Clonazepam and the 7 test compounds are all neutral compounds, therefore they were expected to all lie along the same lane (middle lane) as the parent compound Clonazepam. Since the chemical and structural variation among the group was minimal, the 7 test compounds were expected to cluster within the same chemical space. The 11 test compounds were divided into two subsets according to whether the core benzodiazepine ring structure had a nitro (NO₂) or chloro (Cl) substituent. The rest of the analysis consisted in comparing the other structural and chemical features of the compounds, and evaluating qualitatively and quantitatively their effect on IAM retention of the compounds, and thus their position on the graph.

Subset A: Nitro Substituent

Nitrazepam (1). The chemical features that distinguish Clonazepam from Nitrazepam is the additional Cl moiety in ortho position on the phenyl group. The electon donating character (resonance participation) of the chloro group imparts a slight basicity to the molecule. Therefore, Nitazepam, lacking the chloro substituent on the phenyl group, was predicted to have slightly lower retention on both ^{ester}IAM.PE^{C10/C3} and ^{ester}IAM.PS^{C10/C3} than Clonazepam.

Flunitrazepam (2). The chemical features of Flunitrazepam are close to that of Clonazepam, the additional CH₃ group being the main difference; and it should line up along with Nitrazepam.

Subset B: Chloro Substituent

The compounds in subset B were expected to shift to the right (toward a region of slightly higher IAM retention on the graph), relative to compounds in subset A, due to the electron donating (resonance) character of Cl.

Desmethyldiazepam (3) was expected to have a slight increase in basicity due to the electron donating group Cl, which replaced NO₂ on the Benzodiazepine bicyclic unit. Therefore it was predicted to have longer retention time on ^{ester}IAM.PS^{C10/C3} and ^{ester}IAM.PE^{C10/C3}. Compounds Diazepam (5) and Oxazepam (6) were also expected to have a slight increase in basicity due to the donating groups CH₃ and OH respectively. Therefore both of them were predicted to have longer retention times than Clonazepam on ^{ester}IAM.PS^{C10/C3} and ^{ester}IAM.PE^{C10/C3}. Similarly, Lorazepam (4) and Temazepam (7) were expected to fall in the same region as Diazepam (5) and Oxazepam (6), due to the presence of electron donating groups, (Cl and OH for Lorazepam, and OH and CH₃ for Temazepam).

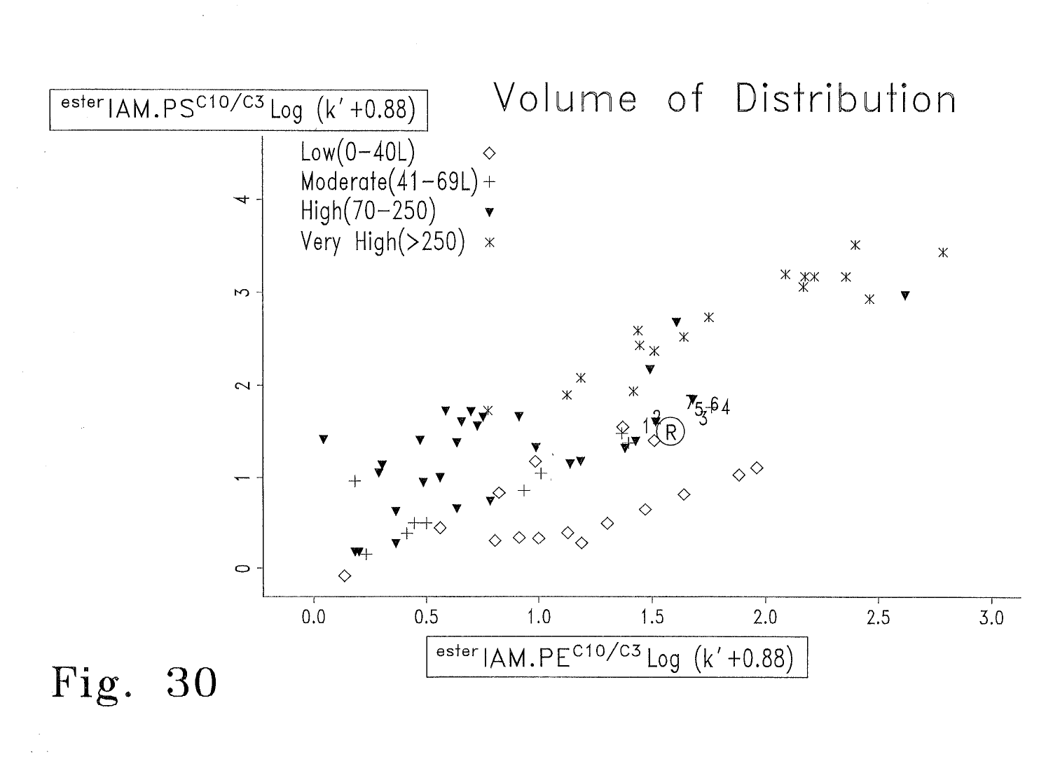

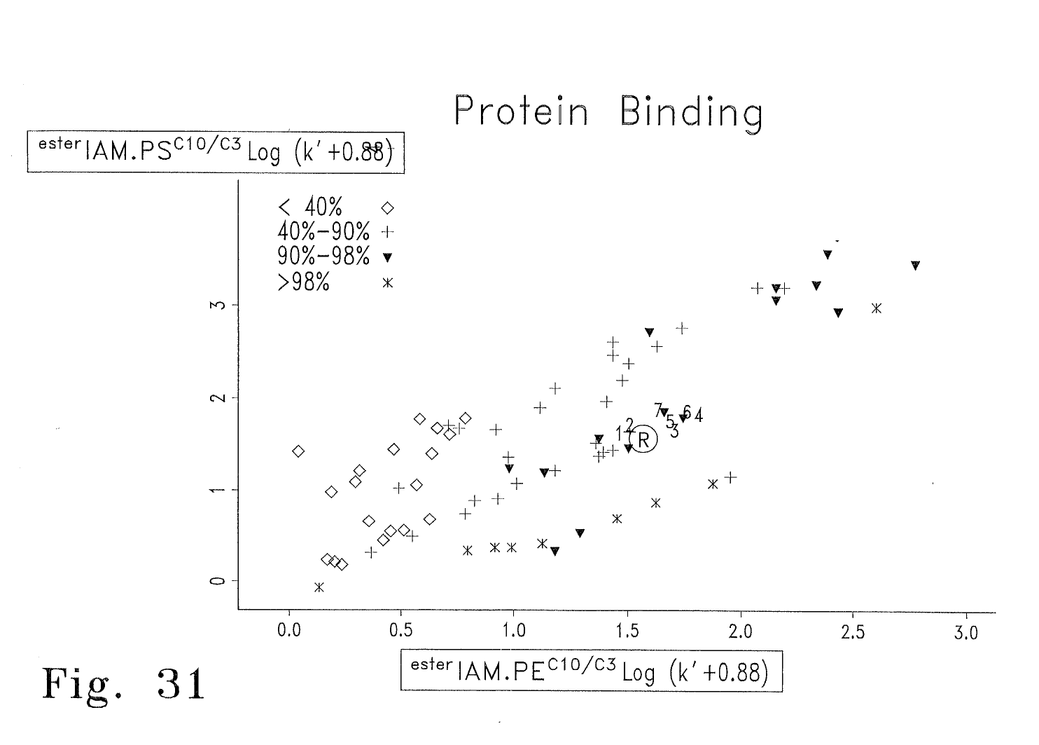

Once the positions of the 7 test compounds had been predicted on the general graph, the information was overlaid onto the three ADME standard curves, as depicted on FIGS. 29-31. The ADME properties of each compound were predicted according to the area on the three standard ADME curves where each compound fell. The results are summarized in Table 5.

TABLE 5

| | | | | Volume of | | % Protein | |
|---|---|---|---|---|---|---|---|
| Compound | | Clearance | | Distribution (L) | | Binding | |
| # | Compound Name | (Ref.) | Prediction | (Ref.) | Prediction | (Ref.) | Prediction |
| R | clonazepam | low | — | 210 | — | 86 | — |
| | (Ref.) | | | | | | |
| 1 | nitrazepam | low | low | 133 | high | 87 | 40-89 |
| | | | | (high) | | | |
| 2 | flunitrazepam | low | low | 231 | high | 77 | 40-89 |
| | | | | (high) | | | |
| 3 | desmethyldiazepam | low | low | 54.6 | high | 97 | 90-97 |
| | | | | (moderate) | | | |
| 4 | lorazepam | low | low | 91 | high | 91 | 90-97 |
| | | | | (high) | | | |
| 5 | diazepam | low | low | 77 | high | 98 | 90-97 |
| | | | | (high) | | | |
| 6 | oxazepam | low | low | 42 | high | 98 | 90-97 |
| | | | | (moderate) | | | |
| 7 | temazepam | low | low | 66.5 | high | 97 | 90-97 |
| | | | | (moderate) | | | |

| | | | |
|---|---|---|---|
| Prediction success rate | 100% (7/7) | 57% (4/7) | 100% (7/7) |
| (Strict) | | | |
| Prediction success rate | | 100% (7/7) | |
| (Close calls) | | | |

The experimental error associated with the determination of ADME data is significant. ADME data from one individual to the next vary greatly, thus the reference values given in Table 5 are not really hard numbers. The ADME data should be expressed as a range, reflecting the fact that the data varies from one individual to the next, and depends on the conditions under which it was collected. Thus reference ADME data for each compound is a range around the reference values given in Table 5. Based on these considerations, it is reasonable to deem the predictions in lightly shaded boxes acceptable.

The prediction success rates were very encouraging. However, the above study only illustrated the method for a group of structural analogs. The method was tried on a chemically diverse group in Study 2.

Study 2

A group of test compounds was generated from a similarity search based on a parent compound (Atenolol). The Membrane Affinity Fingerprint (MAF) on two IAM surfaces (^{ester}IAM.PE^{C10/C3} and ^{ester}IAM.PS^{C10/C3}) and ADME properties of Atenolol were known. The objective was to estimate the MAFs of the structurally similar compounds based on their structural and chemical features, and predict their ADME properties using the three aforementioned standard curves for clearance, volume of distribution and protein binding. In contrast to Study 1, the standard curves (FIGS. 10-12) were established with 116, 122, and 109 drugs of known for clearance, volume of distribution, and protein binding, respectively. The ADME prediction consisted of assigning each hit compound to the correct category for each ADME property:

| Clearance (CL): | Low | | High | |
|---|---|---|---|---|
| | (<0.378 L/min) | | (>0.378 L/min) | |
| Volume of Distribution (Vd): | 0-40 L | 41-69 L | 70-250 L | >250 L |
| Protein binding: | <40% | 40-89% | 90-97% | >97% |

The similarity search was conducted with ChemFinder on a library of about 80 chemically diverse compounds.

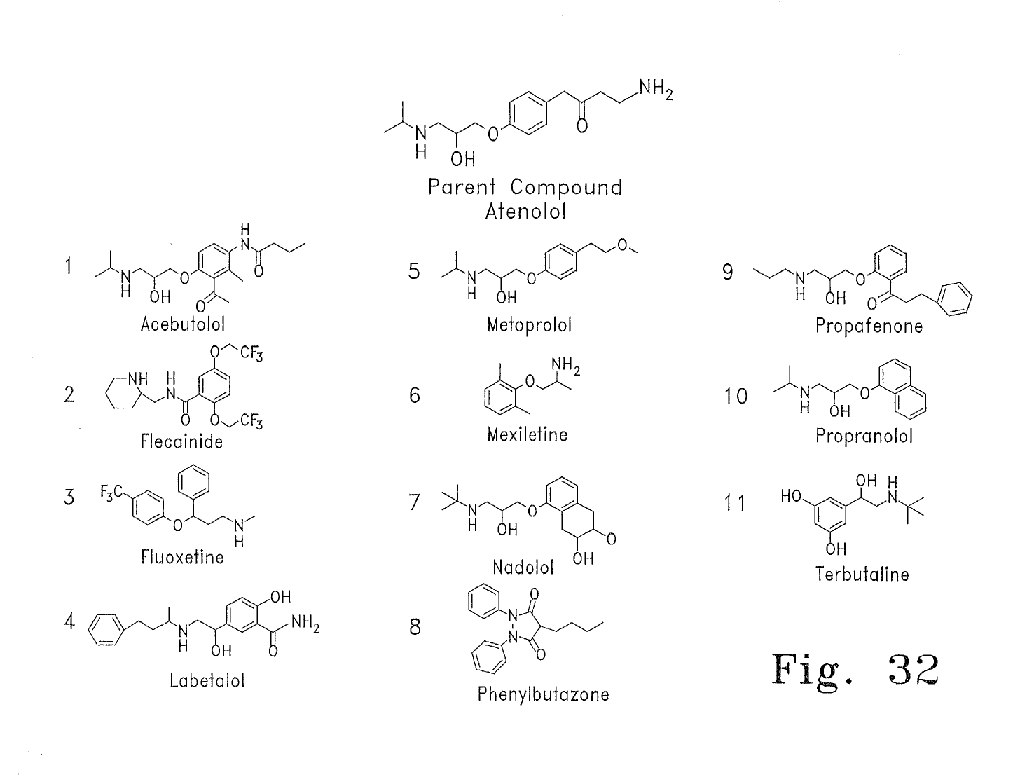

The search identified 11 hit compounds structurally related to Atenolol with various degrees of similarity (65% and above): Acetutolol, Flecainide, Fluoxetine, Labetalol, Metoprolol, Mexiletine, Nadolol, Phenylbutazone, Propafenone, Propranolol, and terbutaline. The chemical structures of the compounds are shown on FIG. 32.

The group of compounds comprised mostly basic mono- and di-basic derivatives, and one neutral compound. The size and shape of the molecules were diverse: from monocyclic to tricyclic, linear or Y-shaped. The structural diversity of the group was such that it covered a wide range in lipophilicity (from significantly polar to fairly greasy).

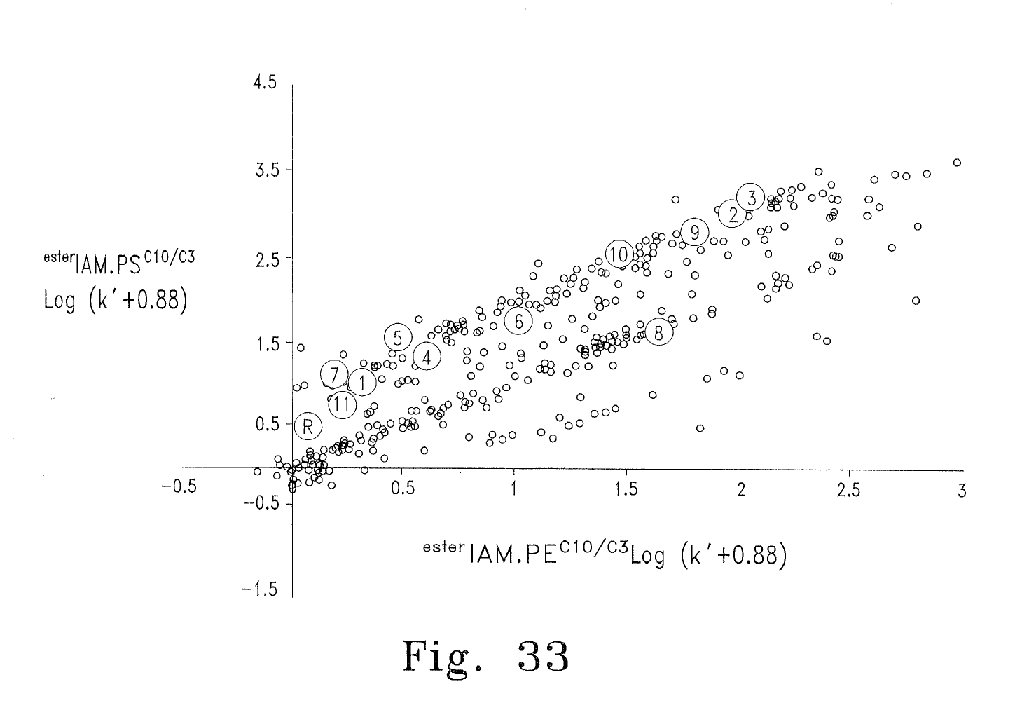

The coordinates of the reference compound Atenolol on the general graph (see FIG. 13, data point labeled “R”) were known: its capacity factors on ^{ester}IAM.PE^{C10/C3} and ^{ester}IAM.PS^{C10/C3} had been determined previously. Each data point on the graph shown on FIG. 13 represents a drug with a known MAF on ^{ester}IAM.PE^{C10/C3} and ^{ester}IAM.PS^{C10/C3}. This graph represents the chemical space covered by approximately 400 drugs. The ADME properties of ˜100 of these 400 drugs were known. These 100 compounds were used to generate the standard curves for volume of distribution, protein binding, and clearance (FIGS. 10-12). The ADME properties of the parent compound were also known: CL=0.140 L/min, Vd=67 L, and Protein binding=5%. Each of the 11 hit compounds was positioned on the general graph, relative to Atenolol and to one another, according to structural and chemical criteria (FIG. 33). These criteria included (but are not limited to) Acid/Base chemistry, number of heteroatoms, polarity, number of rings, lipophilicity, ability to form intra- and intermolecular hydrogen bonds, number of unsaturations, topology and size of the molecules.

With the exception of Phenylbutazone, which is neutral, all the compounds were basic (like Atenolol). Therefore they were all predicted to fall on the same lane as Atenolol on the graph (upper lane). The rest of the analysis consisted in comparing the other structural and chemical features of the compounds, and evaluating qualitatively and quantitatively their effect on JAM retention of the compounds, and thus their position on the general graph.

Detailed Analysis

The chemical features that distinguish Acebutolol from Atenolol are its amide and acyl groups, which are not nearly as polar as the aminocarnonyl group of Atenolol. Therefore, Acebutolol was predicted to have slightly higher retention on both ^{ester}IAM.PE^{C10/C3} and ^{ester}IAM.PS^{C10/C3} than Atenolol, as shown on FIG. 33.

Flecainide is a secondary amine, like Acebutolol and Atenolol, but it features two trifluoromethyl group and a piperidinyl group which should impart significant lipophilicity to the compound. Consequently, Flecainide should have a significantly higher affinity for both IAM surfaces. Based on these observations, Flecainide was predicted to be pushed way up the graph, relative to the parent compound Atenolol, and was positioned as shown on FIG. 33.

Because of its two phenyl groups and its trifluoromethyl group, Fluoxetine should be fairly greasy. Like Flecainide, it also has a secondary amine. However, in the case of Fluoxetine, intramolecular hydrogen bonding between the amino group and the ether group is possible, making the compound less polar than a free amine like Flecainide. Therefore, the IAM retention of Fluoxetine was estimated to be slightly higher than Flecainide.

Labetalol is expected to be more lipophilic than Acebutolol and Atenolol, due to its phenylbutyl side chain. However the presence of polar groups such as the two hydroxyls, the secondary amine and the aminocarbonyl impart a certain degree of polarity to the molecule, so that the shift toward the lipophilic region on the graph will not be as significant as propafenone (see discussion below) which lack the polar groups of Labetalol.

Metoprolol features the same hydroxy-N-isopropyl-propyloxy side chain as Atenolol and Acebutolol. However, due to its less polar methyl-ethyl ether side chain, Metoprolol is expected to be relatively more lipophilic than Atenolol but slightly less polar than Acebutolol. Based on these considerations, the compound was estimated to move slightly up the upper lane relative to Acebutolol.

Mexiletine is a relatively small molecule with a primary amine, and an ether oxygen shielded by two ortho methyl groups. The molecule is expected to fall in the region of moderate lipophilicity as shown on the graph on FIG. 33.

Nadolol, with a total of three hydroxyl groups, appears to be fairly polar and is expected to remain in the chemical space neighboring that of Acebutolol and Atenolol.

Phenylbutazone is a neutral compound, therefore it is predicted to fall in the “neutral lane” (middle lane), as shown on FIG. 33. The polarity of the molecule relies solely on the presence of two very deshielded amide groups (N-phenyl amides) with very poor basicity and nucleophilicity. On the other hand, its two phenyl groups and butyl side chain impart a fair amount of lipophilicity to the compound. The molecule is expected to be fairly greasy, and is predicted to fall in the region with moderate to high IAM retention, as shown on FIG. 33.

The phenylpropanone side chain of Propafenone imparts a considerable amount of lipophilic character to the molecule. In contrast to Labetalol, Propafenone does not have additional polar substituents such as a hydroxy or aminocarbonyl group, which would counteract the effect of the phenylpropanone group. Therefore Propafenone was predicted to significantly be pushed up the lane, relative to more polar compounds such as Acebutolol, Atenolol, and Labetalol.

The naphthalene moiety of Propranolol will impart some degree of lipophilicity to the molecule, but the increase in lipophilicity is expected to be less significant than that of two phenyl groups (such as in the case as Propafenone). The aromaticity in Propranolol is “localized” in a single naphthalene unit, as opposed to being spread over the molecule into two independent aromatic units (phenyl groups). Furthermore, Propranolol has 5 carbons less than Propafenone. Overall, the compound was rated significantly more greasy than Acebutolol, Atenolol, and Labetalol, but slightly less lipophilic than Propafenone. Based on these considerations, Propranolol was predicted to fall in a region of fairly high lipophilicity.

Terbutaline is a monoaromatic compound with three hydroxy groups and a secondary amine group. Despite the presence of a tert-butyl substituent on the amine, the molecule is overall very polar and is expected to reside in the polar region of the graph. Terbutaline is predicted to fall in the neighboring region of Atenolol.

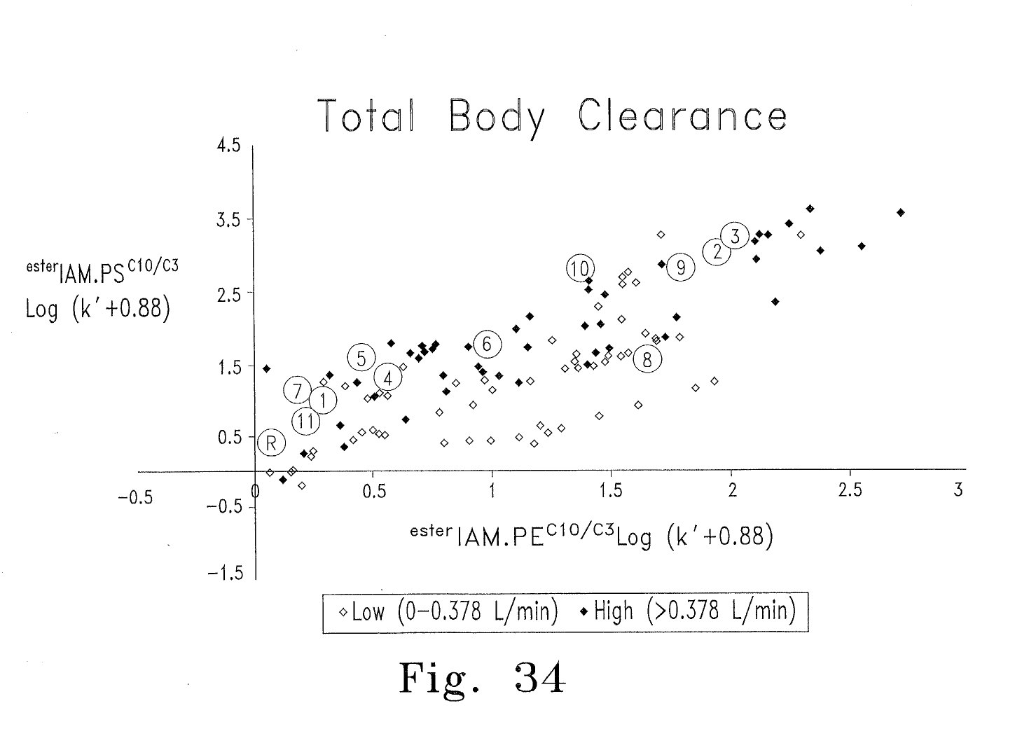

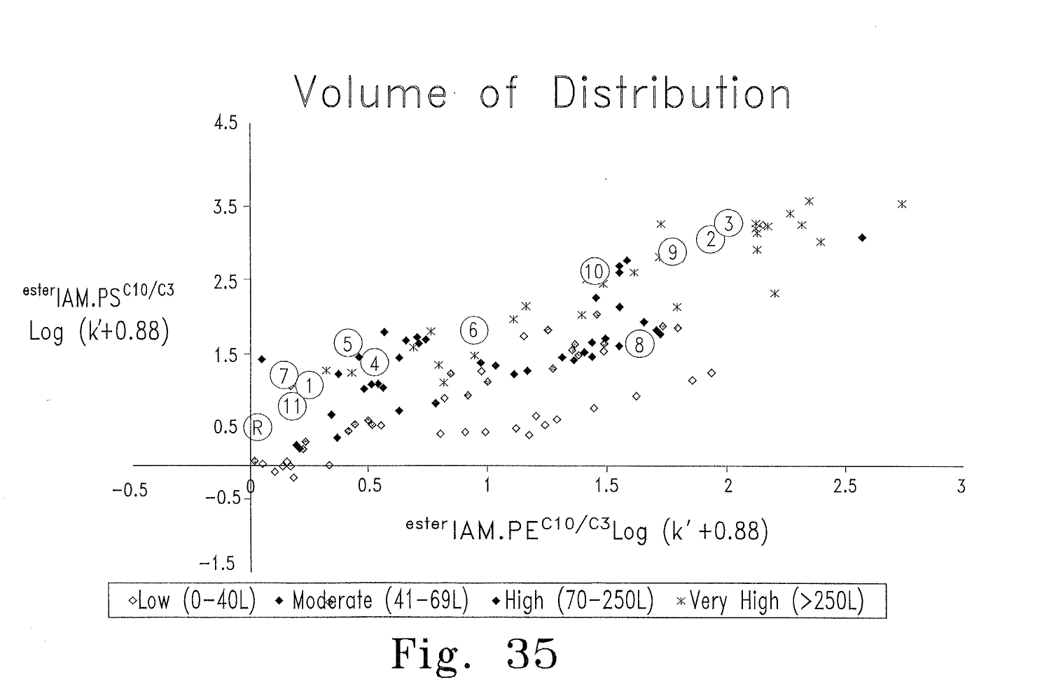

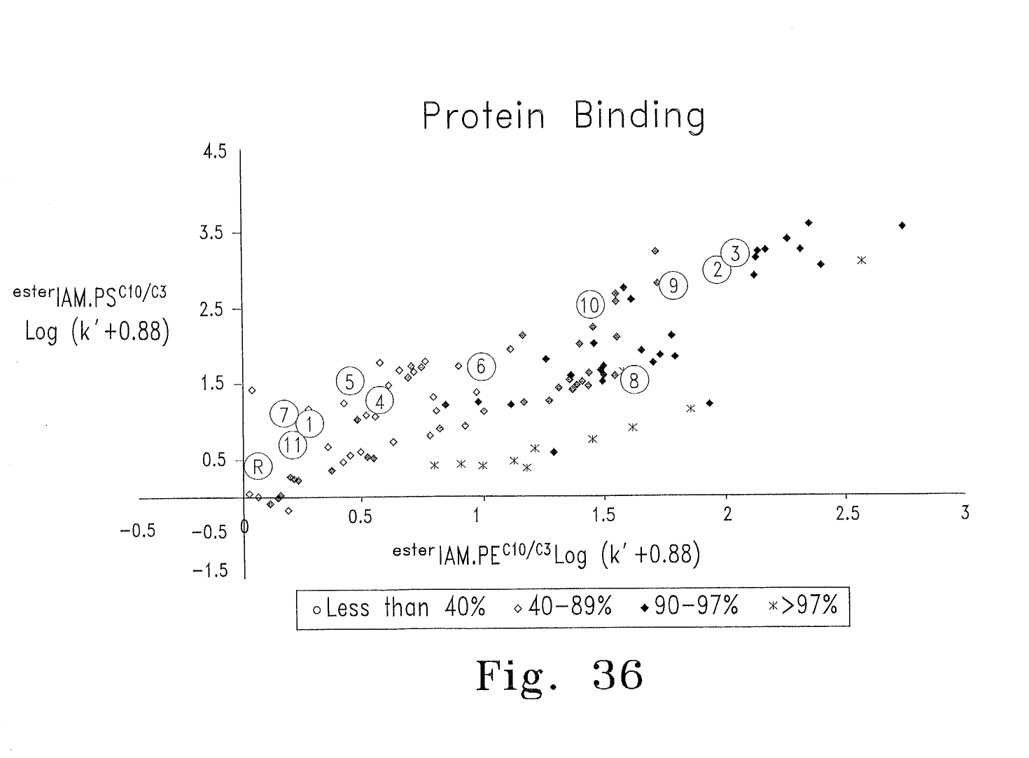

Once the positions of the 11 test compounds had been predicted on the general graph, the information was overlaid onto the three ADME standard curves, as depicted on FIGS. 34-36. The ADME properties of each compound were predicted according to the area on the three standard ADME curves where each compound fell. The results for Protein Binding and Volume of Distribution are summarized in Table 6.

TABLE 6

| | | Volume of | | % Protein | |
|---|---|---|---|---|---|
| Compound | | Distribution (L) | | Binding | |
| # | Compound Name | (Ref.) | Prediction | (Ref.) | Prediction |
| R | atenolol (parent | 67 | | 5 | |
| | compound | | | | |
| 1 | acebutolol | 84 | 70-250 | 26 | <40 |
| 2 | flucainide | 343 | /250 | 61 | ˜90 |
| 3 | fluoxetine | 2450 | /250 | 94 | 90-98 |
| 4 | labetalol | 658 | 70-250 | 50 | ˜40 |
| 5 | metoprolol | 294 | 70-250 | 11 | <40 |
| 6 | mexiletine | 343 | 70-250 | 63 | 40-90 |
| 7 | nadolol | 133 | 70-250 | 20 | <40 |
| 8 | phenylbutazone | 7 | 41-69 | 96 | >90 |
| 9 | propafenone | 252 | /250 | 87 | ˜90 |
| 10 | propranolol | 301 | 70-250 | 87 | 40-90 |
| 11 | terbutaline | 126 | 70-250 | 20 | <40 |

| | | |
|---|---|---|
| Prediction success rate | 55% (6/11) | 82% (9/11) |
| (Strict) | | |
| Prediction success rate | 100% (11/11) | 100% (11/11) |
| (Close calls) | | |

Although the prediction models have not been optimized, the prediction rates still turned out fairly high for the ADME properties under consideration. These results are very encouraging, and are evidence that the unconventional approach to in silico predictions described above shows great promise in the field of pharmaceutical development and drug discovery.

### EXAMPLE 8

### Chemical Principles Applied to Calibration Curves

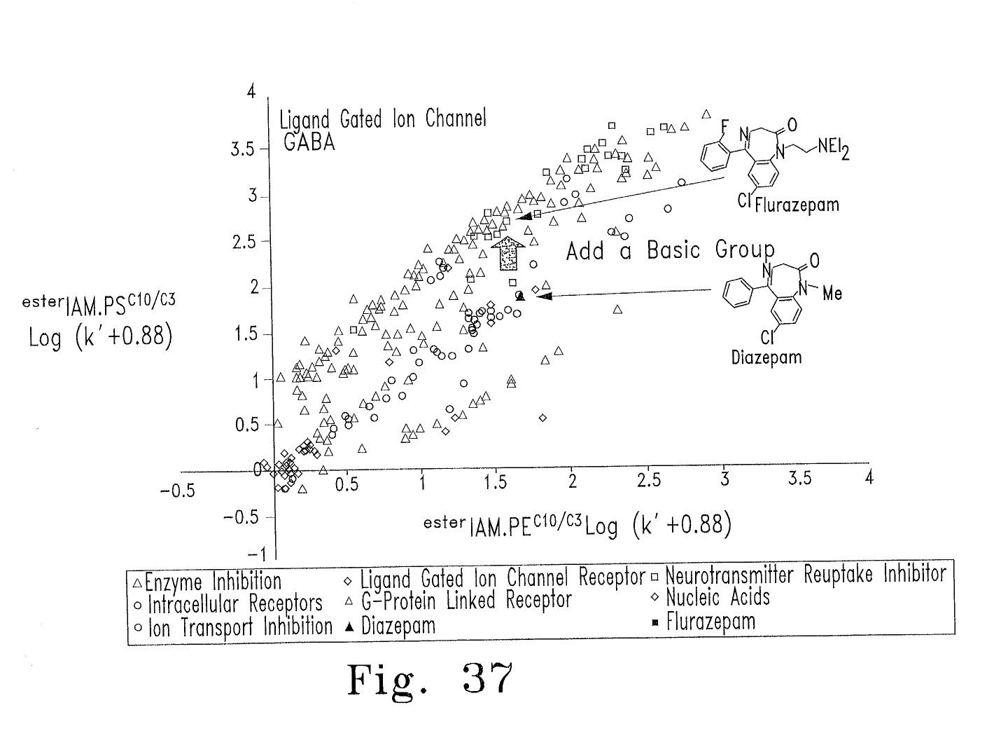

FIG. 18 is a plot of approximately 400 drug substances with membrane binding properties measured on ^{ester}IAM.PE^{C10/C3} (x-axis) and ^{ester}IAM.PE^{C10/C3} (y-axis). This Taxonomy plot represents the membrane binding constants of virtually all compounds in the MAF database. FIG. 1 shows three approximately linear lanes. The upper most lane constitutes basic compounds and the lowest lane acidic compounds, and the middle lane neutral compounds. Thus one can predict that changing a neutral compound into a basic compound by structural modification will shift the position of the compound. FIG. 37 demonstrates this for Diazepam, a neutral compound, and Flurazepam, which is a basic compound. Furthermore, since there was little change in the hydrophilicity or lipophilicity of the test compounds, a vertical displacement from the middle lane to the basic lane was predicted. It is appreciated that commercial and non commercial algorithms that calculate molecular descriptors including lipophilicity, hydrophobicity, polarizability, etc. can be used to obtain estimates of molecular properties that can be used to predict where test compounds will reside on the graph.

### EXAMPLE 9

### Chemical Principles Applied to Calibration Curves

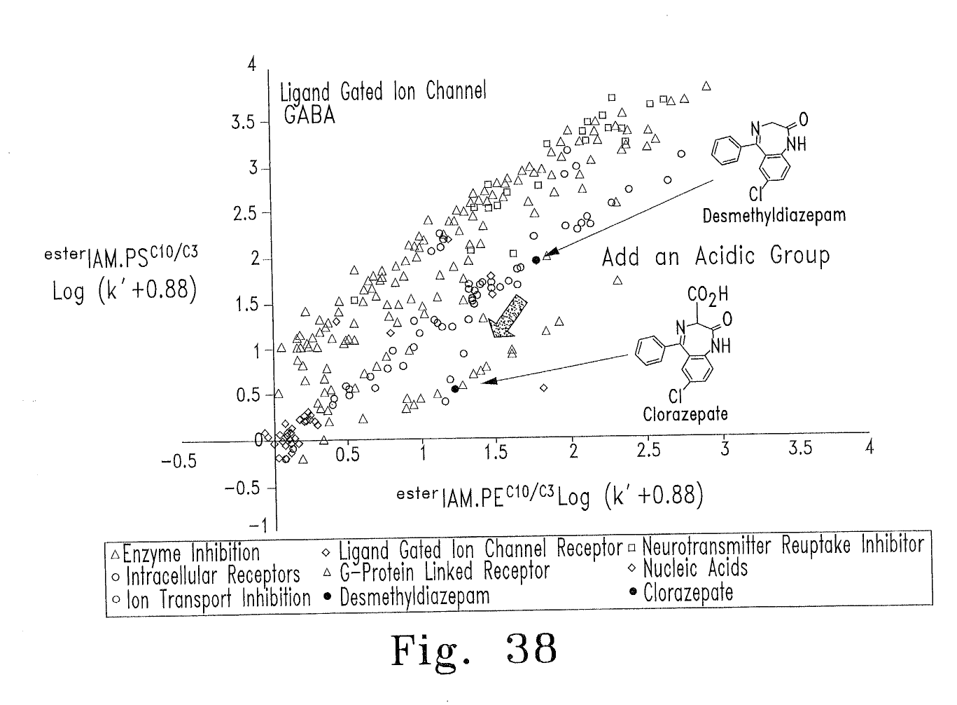

FIG. 38 is conceptually testing the same principle as FIG. 37, i.e., that changing the charge on a molecule shifts position of the molecule on calibration curves prepared from membrane binding and/or related data. However in this case the polarity of the molecules significantly differ, and it affects the predictions. Thus the neutral compound Desmethyldiazepam is in the neutral lane whereas adding a carboxyl group to form Clorazepate imparts not only a net negative charge to the molecule at physiological pH, but also increases the hydrophilicity. The binding of Clorazepate to ^{ester}IAM.PS^{C10/C3} surfaces (y-axis) is thus expected to decrease for two reasons: (1) negatively charged solute molecules will be repelled by the negatively charged IAM surface, and (2) the molecule is also more polar and therefore more soluble in the mobile phase. Similarly, the binding of Clorazepate to ^{ester}IAM.PE^{C10/C3} surfaces (x-axis) is thus expected to decrease because of the increased polarity. In general, under the test conditions used to generate the data set, increasing the hydrophobicity increases retention on all surfaces and increasing the polarity tends to decrease the retention of compounds on all surfaces.

### EXAMPLE 10

### Chemical Principles Applied to Calibration Curves

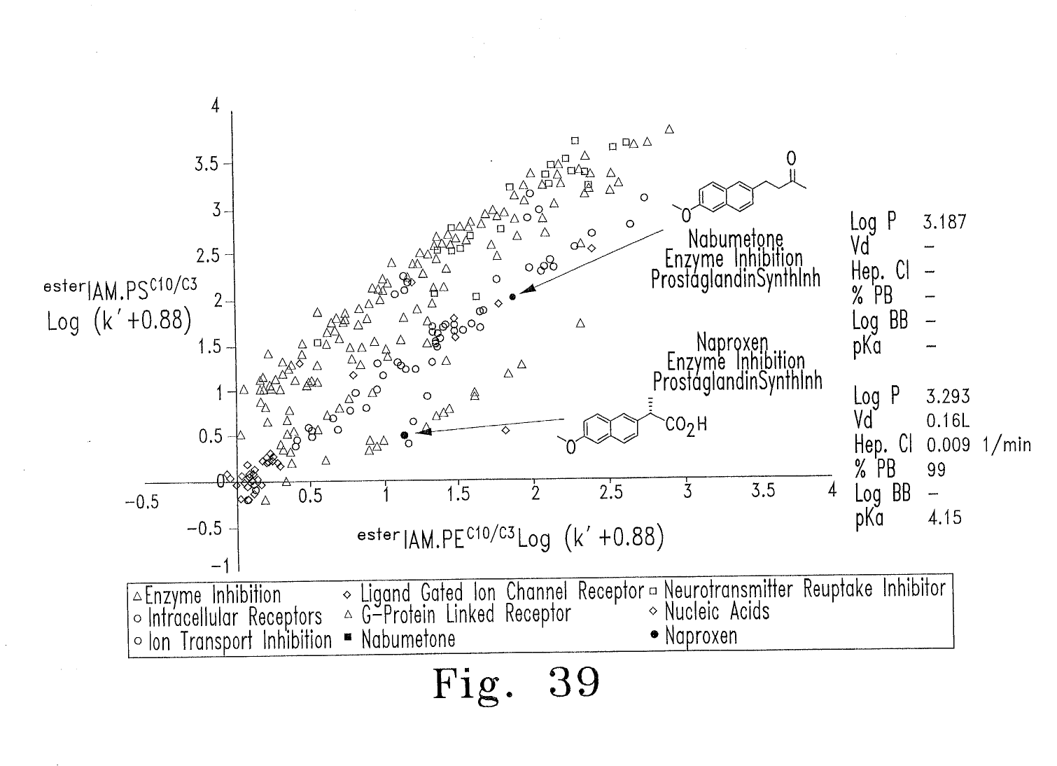

This is another example demonstrating the chemical principles described in Example 9 with the exception that it utilizes different test compounds. Nabumetone is a neutral hydrophobic molecule, residing in the neutral lane of the calibration curve shown in FIG. 39, whereas the highly structurally similar molecule Naproxen is in the acidic lane closer to the origin, which reflects its decreased affinity to both IAM surfaces that were used to generate the calibration plot.

### EXAMPLE 11

### Protein Binding Calibration Curve

FIG. 10 is a plot of approximately 80 drug substances with membrane binding properties measured on ^{ester}IAM.PE^{C10/C3} (x-axis) and ^{ester}IAM.PS^{C10/C3} (y-axis). As shown in FIG. 10, the human protein binding data can be grouped into 4 subgroups with some overlap among the groups. Molecules on this graph can be grouped into one of the following categories: (1)<40%, (2) 40-89%, (3) 90-97%, and (4)>97%. Because the molecules cluster into 4 groups this graph can be used as a calibration curve to predict the protein binding of compounds according to the principles described in Examples 8-10. Similar to the calibration curve shown in FIG. 10, there are three discernible lanes in the calibration curve and these lanes cluster basic compounds (upper lane), neutral compounds (middle lane) and acidic compounds (lower lane).

### EXAMPLE 12

### Predicting Protein Binding using Chemical Principles

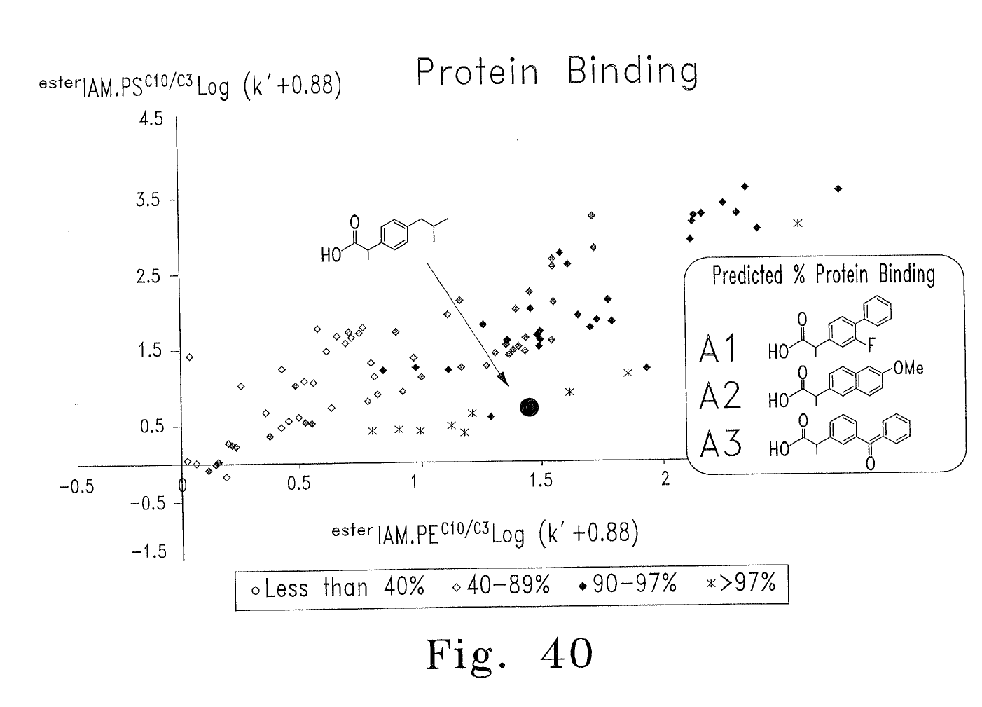

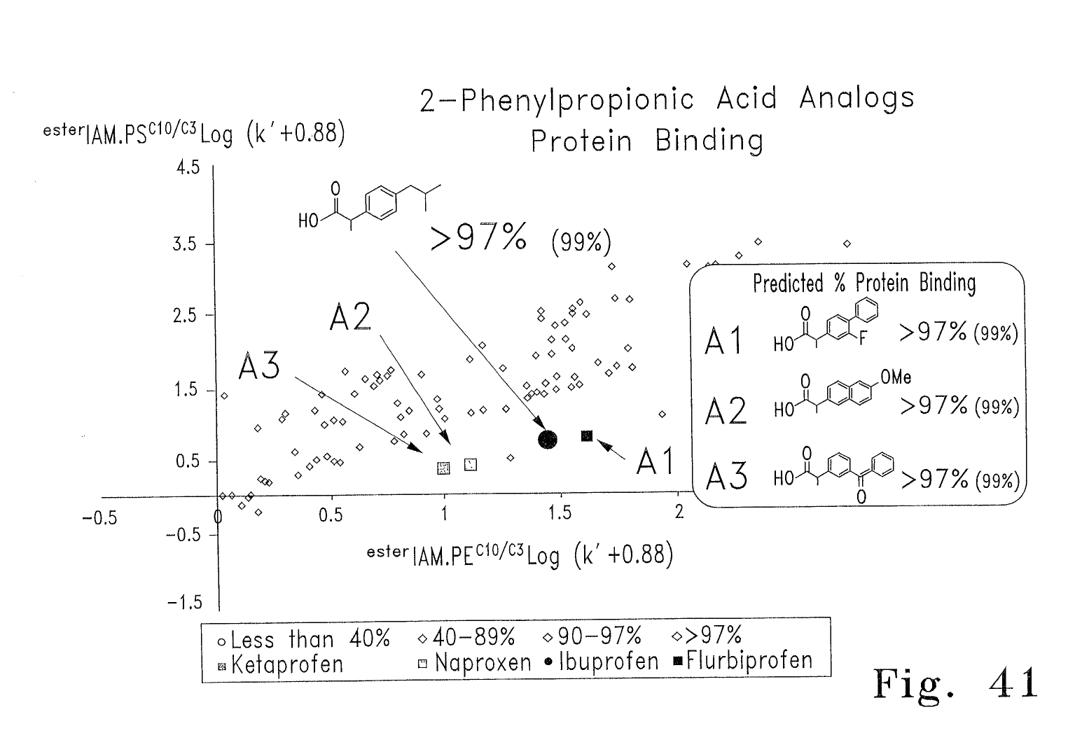

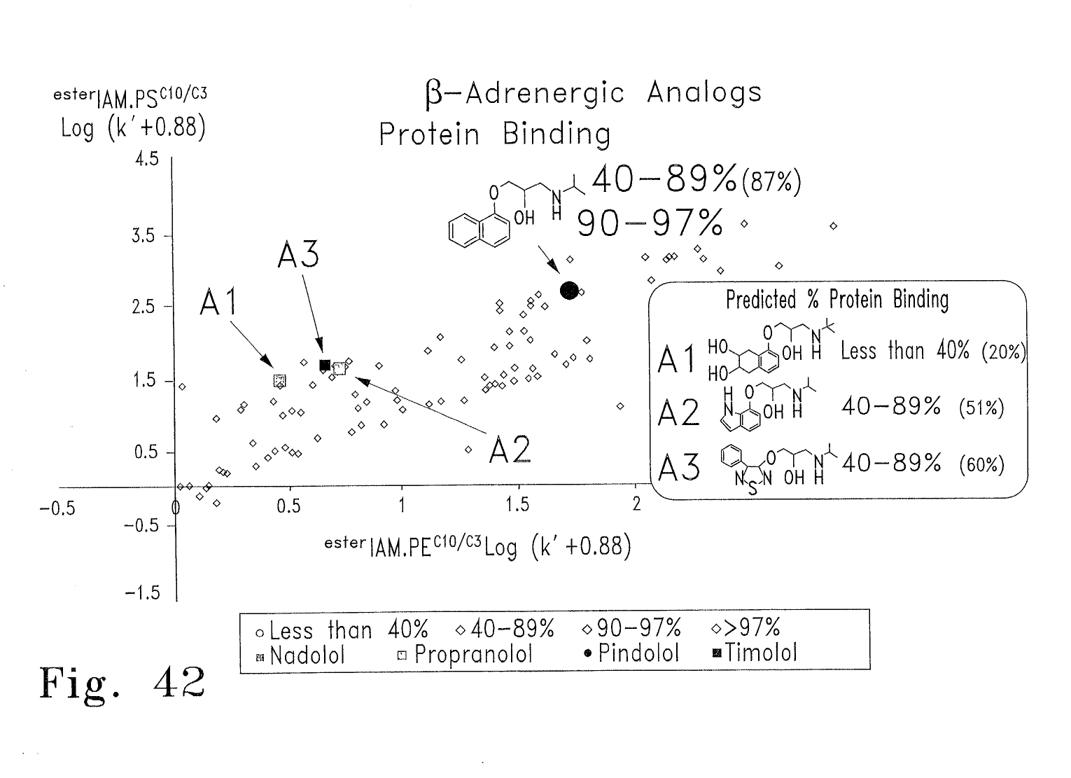

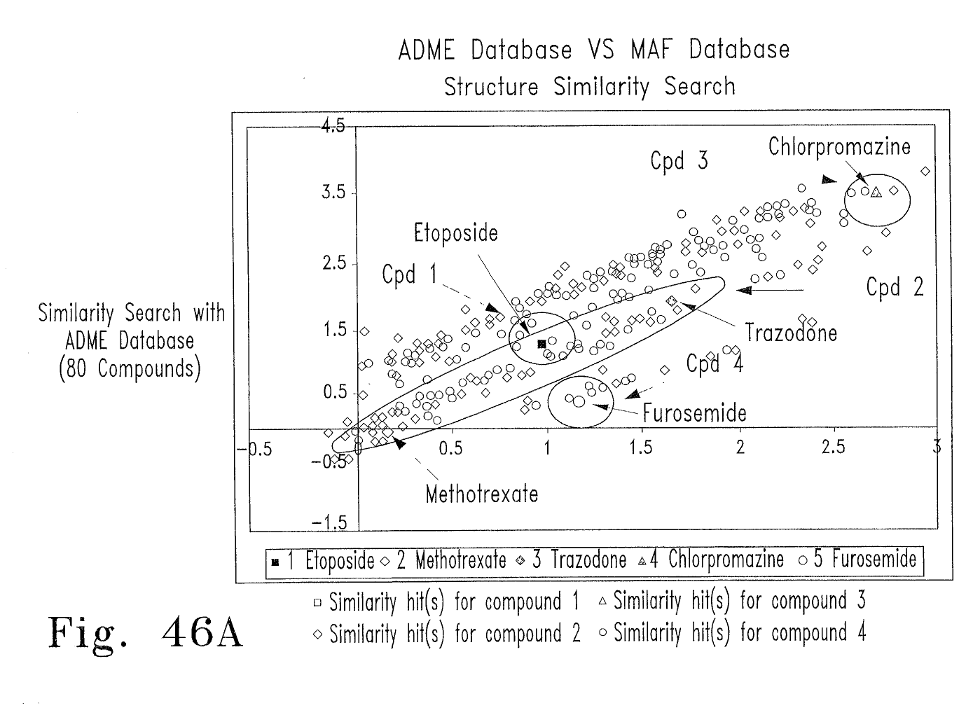

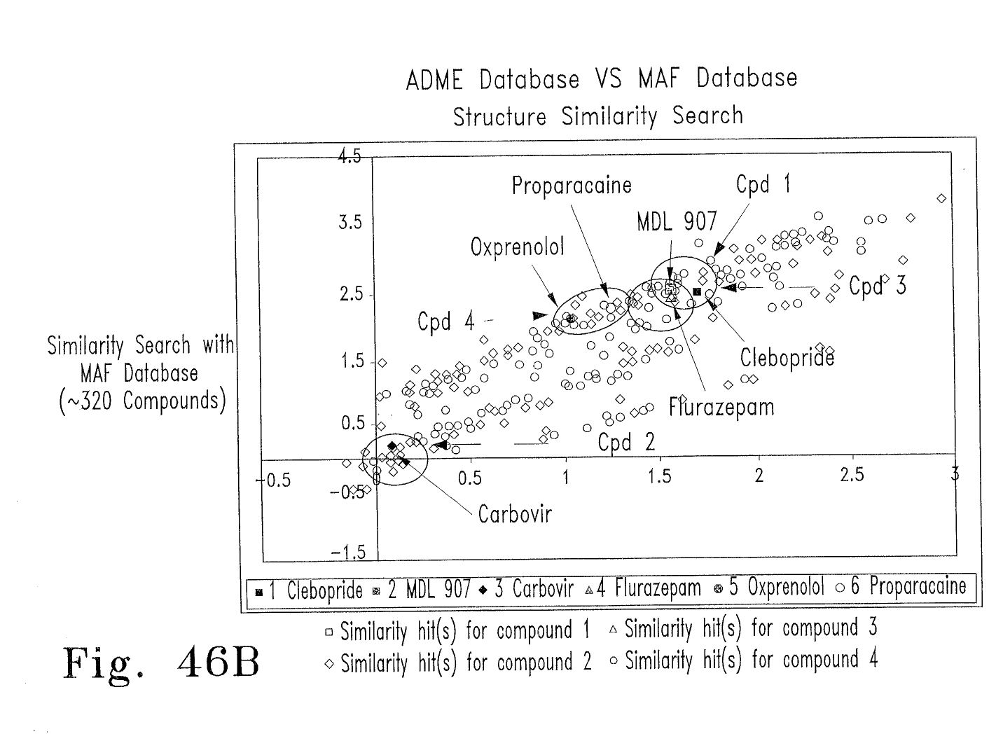

One embodiment of the present invention is to measure the membrane binding properties of a compound or scaffold and design analogs of the scaffold that have preferred pharmacokinetic properties, or at least the pharmacokinetic properties are known to a good approximation prior to making the molecules. This is exemplified in FIG. 40 and FIG. 41 for phenylproprionic acid analogs of Ibuprofen. FIG. 46 places Ibuprofen (a putative scaffold) on the protein binding calibration curve based on empirically derived membrane binding data. Three analogs in FIG. 40 are shown and the objective of the study was to predict where the analogs would fall based on the membrane binding properties of Ibuprofen. All compounds shown in FIG. 40 are acidic and the only difference between the molecules is the degree of polarity. Thus it is expected that all molecules will reside in the acidic lane. Furthermore, the most hydrophobic molecule is A1, which is Flubiprofen which should be placed higher than Ibuprofen in the acidic lane. Fluorinated molecules tend to have increased hydrophobicity based on empirical studies. The other two test molecules, A2 and A3 are slightly more hydrophilic than Ibuprofen and are placed near each other but closer to the origin in the acidic lane. FIG. 41 shows the predicted results of protein binding in the inset. Briefly, since all compounds remained in the acidic lane they all have very high protein binding.

### EXAMPLE 13

### Predicting Protein Binding of B Adrenergics

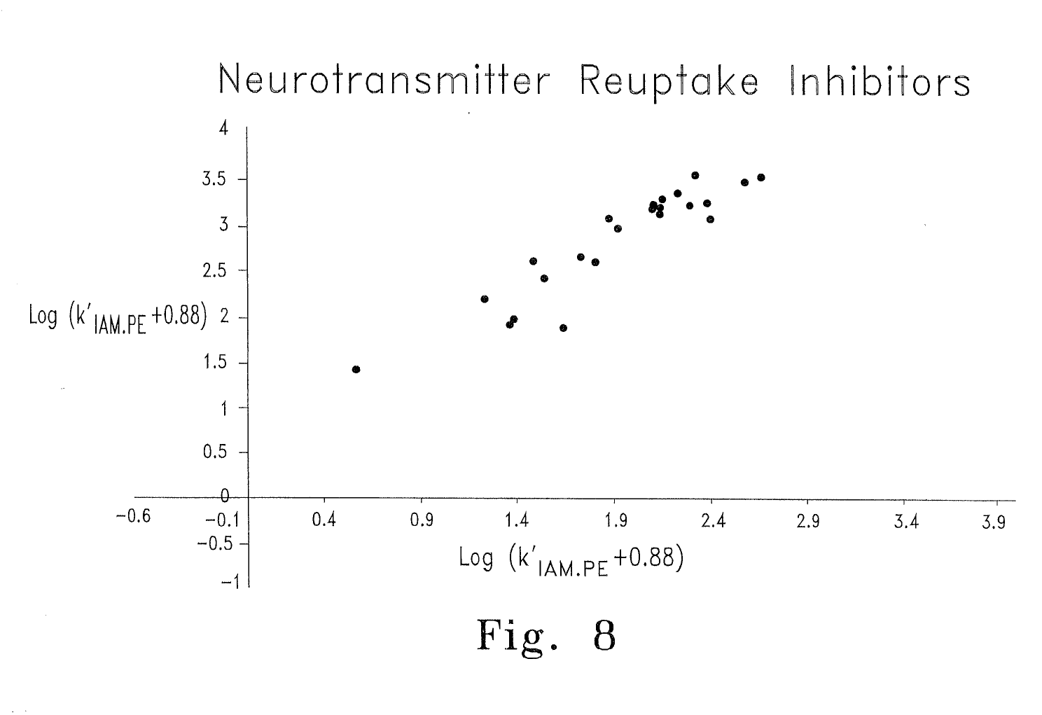

Example 13 is the same prediction-experiment as described in Example 6 with the exception that Propranolol was used as the test compound or scaffold and the membrane binding data of Nadolol, pindolol, and timerol were predicted and used to place these compounds on the graph as shown in FIG. 8. The predicted values are within 10% of the membrane binding data and the protein binding data was correct for all compounds.

### EXAMPLE 14

### Volume of Distribution Calibration Curve

FIG. 14 is a plot of approximately 80 drug substances with membrane binding properties measured on ^{ester}IAM.PE^{C10/C3} (x-axis) and ^{ester}IAM.PS^{C10/C3} (y-axis). As shown in FIG. 11, the human volume of distribution data can be grouped into 4 subgroups with some overlap among the groups. Molecules on this graph can be grouped into one of the following categories: (1) low (0-40 L), (2) moderate (41-69 L), (3) high (70-250 L), and (4) very high (>250 L). Because the molecules cluster into 4 groups this graph can be used as a calibration curve to predict the volume of distribution of compounds according to the principles described above. Similar to the calibration curve shown in FIG. 18, and FIG. 10 there are three lanes in the calibration curve and these lanes cluster basic compounds (upper lane), neutral compounds (middle lane) and acidic compounds (lower lane). Thus the same chemical intuition, used to predict the position of molecules on other calibration curves, apply to the volume of distribution calibration curve shown as FIG. 11.

### EXAMPLE 15

### Predicting Volume of Distribution of B-Adrenergics

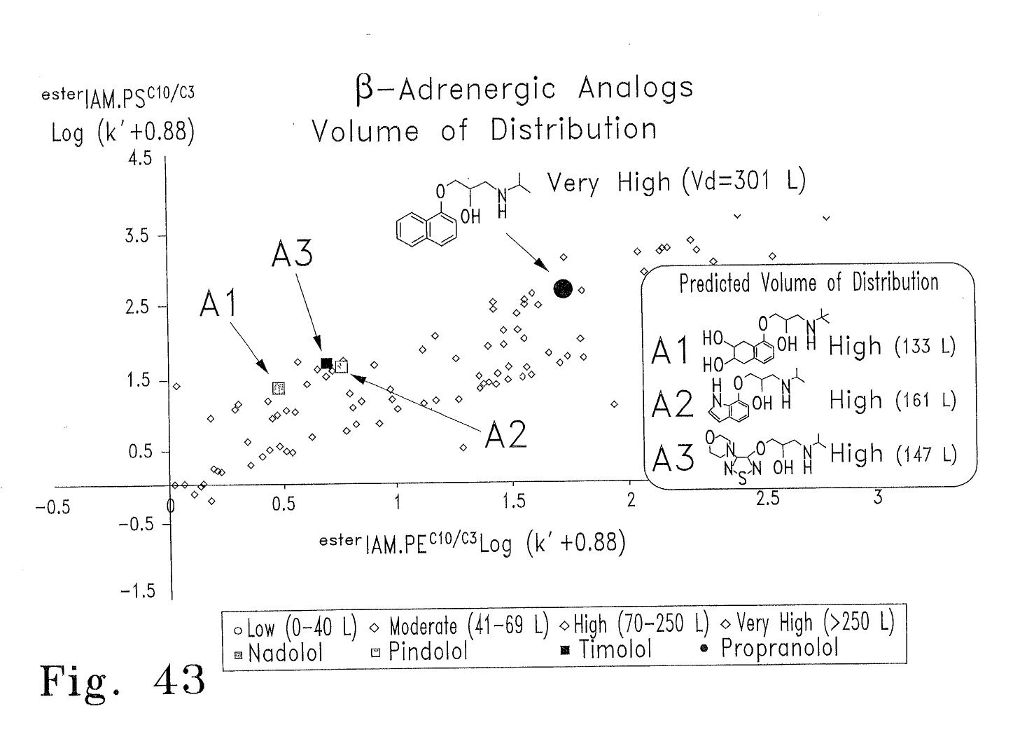

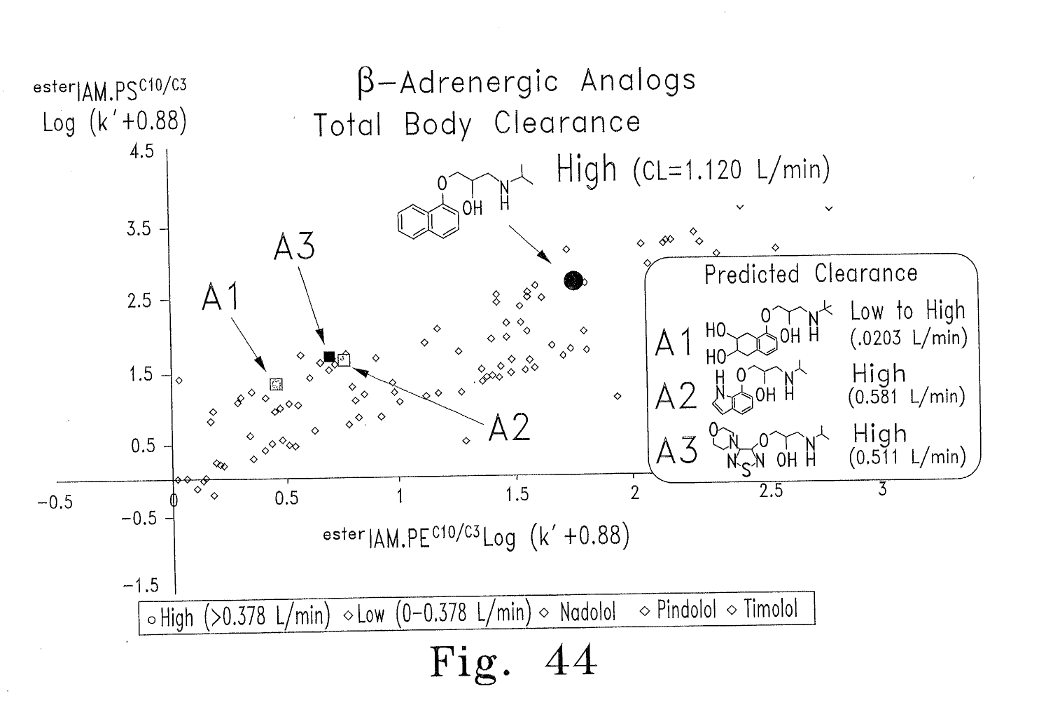

FIG. 43 illustrates the results of an experiment that was performed identical to that described in Example 12 and Example 13 with the exception that volume of distribution is being predicted instead of protein binding. The reference compound, with known membrane binding data was Propranolol, a basic compound. Initially, the membrane binding data of propranolol was placed on the graph and it was predicted to have a very high volume of distribution which was verified from the literature. Next the chemistry of 3 analogs was inspected for changes in charge (there were none, all compounds were basic), increases in hydrophobicity (there were none), and increases in hydrophilicity. One analog (Nadolol) had a gem diol which makes the molecule the most hydrophilic in the series and it was therefore place low in the basic lane. The indole ring of A2 and heterocyclic ring of A3 provide similar polarity and were placed near each other in the graph. All compounds were predicted to have the correct volume of distribution in this example. They were also correctly rank ordered.

### EXAMPLE 16

### Human Body Clearance Calibration Curve

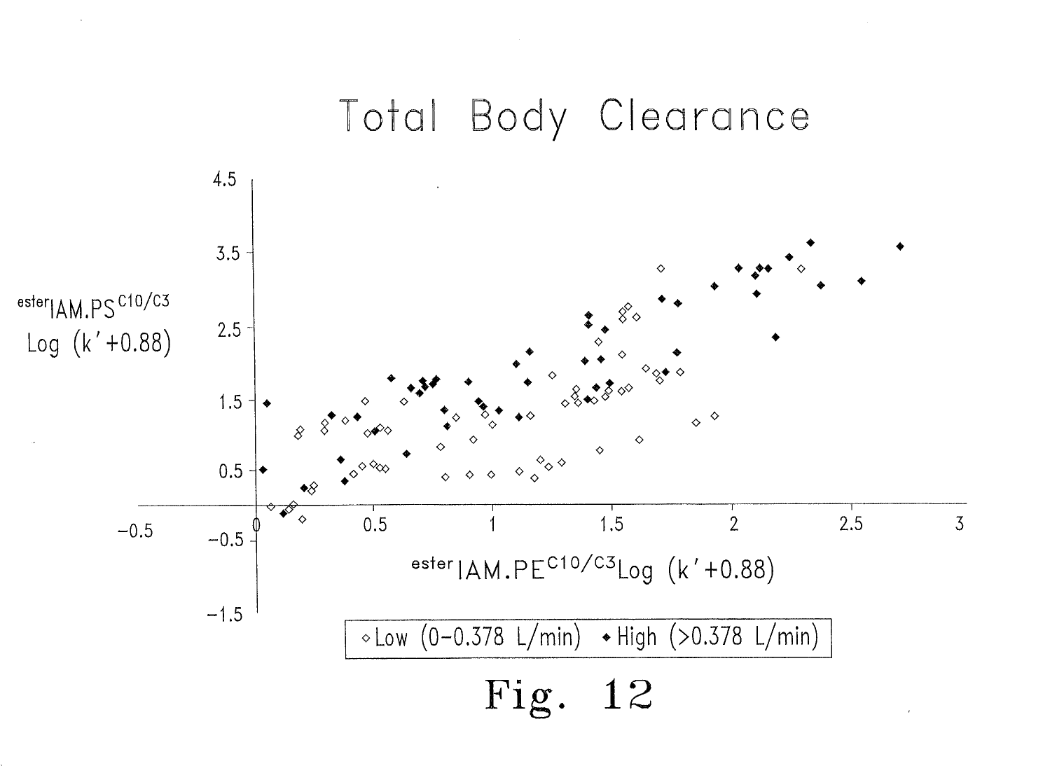

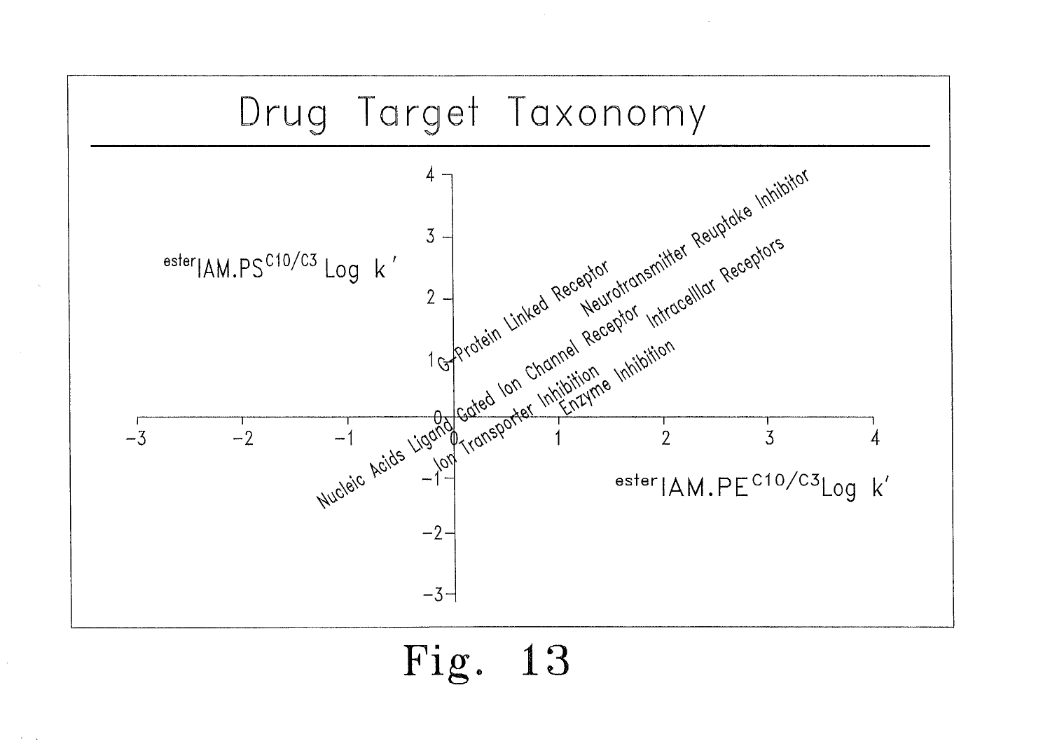

FIG. 12 is a plot of approximately 80 drug substances with membrane binding properties measured on ^{ester}IAM.PE^{C10/C3} (x-axis) and ^{ester}IAM.PE^{C10/C3} (y-axis). As shown in FIG. 12, the human clearance data can be grouped into 2 subgroups with some overlap among the groups. Molecules on this graph can be grouped into one of the following categories: (1) low (0-0.38 L/Min) and (3) high (>0.38L/Min). Because the molecules cluster into 2 groups this graph can be used as a calibration curve to predict the clearance of compounds according to the principles described above. Similar to the calibration curve shown in FIG. 18, FIG. 10, and FIG. 17 there are three lanes in the calibration curve and these lanes cluster basic compounds (upper lane), neutral compounds (middle lane) and acidic compounds (lower lane). Thus the same chemical intuition, used to predict the position of molecules discussed above for the volume of distribution calibration curve and the protein binding calibration curve, can be used to place molecules on the human body clearance calibration curve.

### EXAMPLE 17

### Predicting Clearance of B-Adrenergic Compounds

This is the same experiment as described in EXAMPLE 15, except using the body clearance calibration curve. The same chemical intuition was used to place the B-Adrenergic compounds on the clearance calibration curve relative to propranolol.

### EXAMPLE 18

### Structure Searching ADME Databases and MAF Database

FIG. 18 represents the MAF database whereby the membrane binding of several hundred compounds is plotted. In contrast FIG. 10, FIG. 11, and FIG. 12 are ADME calibration curves used for predicting protein binding, volume of distribution and clearance from chemical intuition. There are only about 80 compounds in each of the ADME calibration plots. Each of the ADME calibration curves were prepared from compounds with known pharmacokinetic properties. Nevertheless, according to the present invention, the key event in predicting ADME properties is to first predict the membrane binding properties. Since the membrane binding properties correlate with ADME properties, the ADME properties can be predicted once the membrane binding properties are known.

Since the primary activity of the present invention, at least with regard to predicting membrane binding data from structures, the best calibration curve for predicting membrane binding is the calibration curve prepared from the entire database, i.e., FIG. 18. In other words, if the initial goal is to predict the membrane binding constants it should be done with the largest dataset, and once the membrane binding constants are obtained, then the ADME can be predicted from known calibration curves that correlate ADME properties with membrane binding properties.

Furthermore, each user of the present invention will have a unique sense/chemical intuition, and prediction results are expected to vary from user to user depending on their sense of how chemical changes in a molecule effect retention on IAM surfaces. To eliminate this problem, it is optimum to use chemical structure searching routines to find molecules on the calibration curves that are closest to the test compounds with unknown membrane binding properties. In other words, an optimum or preferred method for predicting ADME is to (1) use the MAF database as the initial calibration curve for finding membrane binding constants, (2) perform substructure searches of the entire database for each test compound to determine where the test compound should reside on the MAF database calibration curve, (3) adjust the position of the unknown compound if chemical intuition suggests based on structural differences, (4) determine the membrane binding constants of the unknown compound, and (5) predict the ADME from the calibration plots representative of protein binding, volume of distribution, and clearance and any other ADME property or pharmacological property that correlates with membrane binding data. Thus it is a primary objective of the invention to automate, through software, the process of comparing chemicals and predicting ADME.

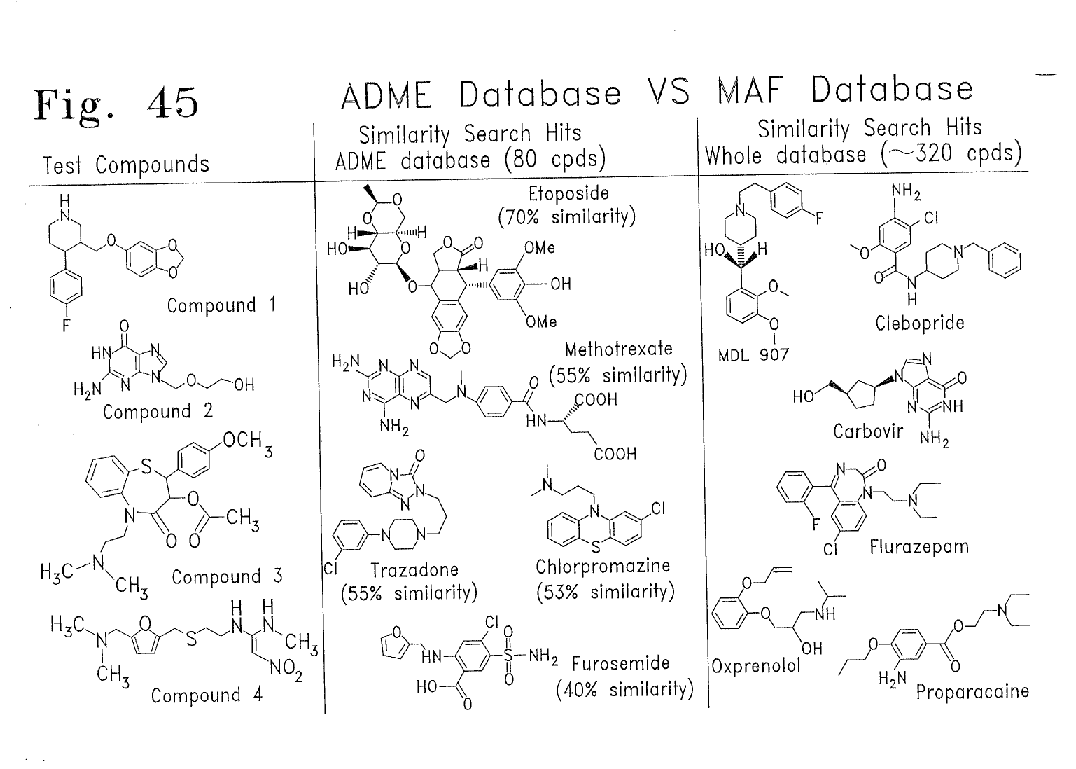

To demonstrate the use of substructure searching and its effect on predicting ADME properties four highly structurally diverse test compounds were chosen to be used as query molecules using CS ChemFinder 4.0 structure search engine. CS ChemFinder 4.0 is commercial software. These query molecules are shown in the left column of FIG. 45. The objective was to compare the membrane binding constants predicted from using only the ADME database or the entire MAF database. The most similar structures to each test compound when searching the ADME database is shown in the middle column of FIG. 45, and the most similar structures to each test compound when searching the MAF database are in the right column of FIG. 45. It is apparent from the FIG. 45 that the right column contains molecules that are more structurally similar to the test compounds than the middle column indicating that it is likely the membrane binding constants predicted from the entire database will be closer to the true values of the test compounds than the compounds in the middle column of FIG. 45.

The results of the query search of the ADME database is shown in the upper graph of FIG. 46. Compound 1 had a closest match to Etoposide which is structurally very different from Compound 1 (FIG. 45) Etoposide is so structurally different compared to Compound 1 that the large ring in FIG. 46 demonstrates where compound 1 may be positioned if only the ADME database is searched for similar structures. Similarly, Compound 2 matched equally well Trazodone and Methotrexate. However Methotrexate was not considered relevant because of the diacid functional group. The position of Trazodone on the MAF database is shown in the upper graph of FIG. 46 and compound 2 would be predicted to be near this compound. The closest structure to compound 3 in the ADME database was chlorpromazine and the upper graph of FIG. 46 shows where compound 3 would be predicted to reside on the MAF database calibration curve. Similarly compound 4 matched furosemide and the upper graph of FIG. 46 shows where compound 4 would reside on the MAF calibration curve. FIG. 46b shows where the 4 test compounds would reside using the structures in the right column of FIG. 45 which are much closer structurally to the test compounds. Note that the entire process was performed using commercial software that searched structure for similar functional groups.

Comparing the FIG. 46a to FIG. 46b, the size of the rings indicate approximately the position of where the test compounds would be predicted to reside if the ADME database was searched (FIG. 46a) versus the MAF database (FIG. 46b). Inspection of the graphs clearly demonstrate that much different results are apparent for each compound. The predictions are reported in Table 7 below. All of the clearance values were predicted correctly when the entire MAF database was used, whereas only 2 of the compounds were correctly predicted when the ADME database was searched. Similarly all of the volume of distributions of the test compounds, were predicted correctly when the MAF database was searched, but only 2 out 4 compounds were correct when the ADME database was searched. Finally, protein-binding results were also better when the membrane binding constants of the entire database were used as the calibration curve.

In conclusion, ADME predictions were better when similarity searches were conducted over the larger pool of compounds in the MAF database with unknown ADME compared to a more restricted search of the ADME database with known ADME properties. This indicates that estimating the membrane binding properties of the compounds is paramount before estimating the ADME properties, at least with regard to automated structure searching routines.

TABLE 7
ADME Database vs. MAF Database

| Clearance | Vd | | | Protein Binding | |
|---|---|---|---|---|---|
| Predicted | | | | | |
| (ADME Database) | | | | | |
| 1 Low | 1 | 40-70 | L | 1 | 40-90% |
| 2 High | 2 | 70-250 | L | 2 | 0-40% |
| 3 High | 3 | 250 | L | 3 | 90-97% |
| 4 High | 4 | 70-250 | L | 4 | 40-90% |
| Observed | | | | | |
| 1 High (0.602 L/min) | 1 | >250 | L (1.190 L) | 1 | 90-97% (95%) |
| 2 Low (0.235/L/min) | 2 | 40-70 | L (48 L) | 2 | 0-40% (15%) |
| 3 High (0.840 L/min) | 3 | >250 | L (217 L) | 3 | 40-90% (78%) |
| 4 High (0.728 L/min) | 4 | 70-250 | L (91 L) | 4 | 0-40% (15%) |
| Predicted | | | | | |
| (MAF Database) | | | | | |
| 1 High | 1 | >250 | L | 1 | 90-97% |
| 2 Low | 2 | <40 | L | 2 | 0-40% |
| 3 High | 3 | >250 | L | 3 | 90-97% |
| 4 High | 4 | 70-250 | L | 4 | 40-90% |

### EXAMPLE 19

### Identifying Compounds with Possible Activity by Combining MAF Data and Structure Similarity Data

All of the above examples that predicted ADME from MAF utilized a single chemical test compound to predict the membrane binding properties from structure comparisons. Thus finding a molecule with known retention on an IAM column that is structurally similar to a compound with unknown retention can be used to predict the membrane binding properties (or retention) of the unknown compound and subsequently the ADME properties of the compound.

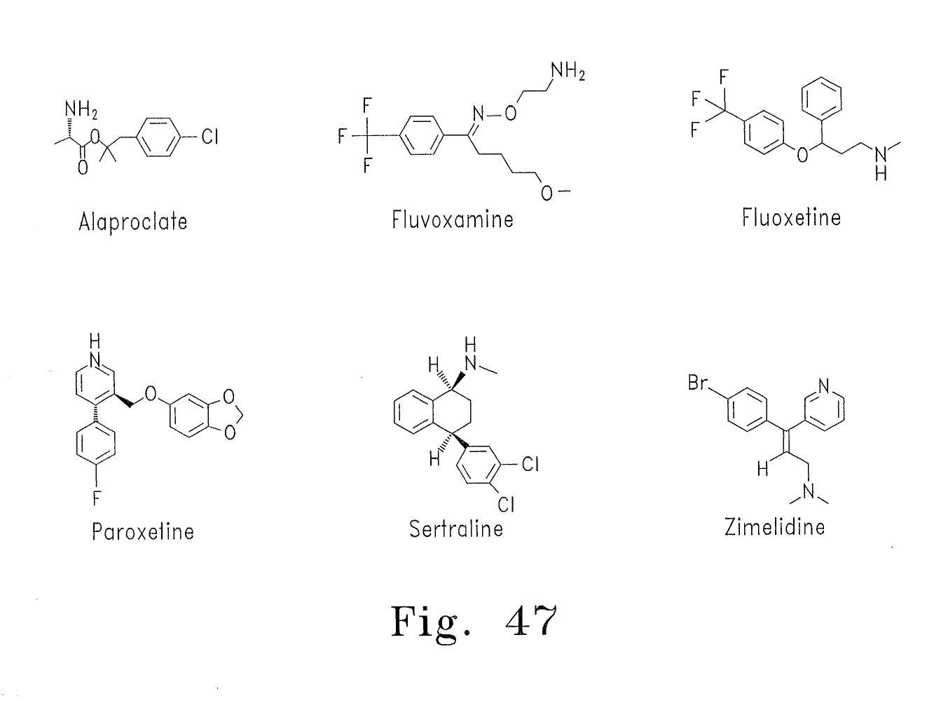

However, it has been shown several times in the art that different compounds which bind to the same receptor frequently have similar membrane binding properties in spite of significant differences in their structures which is exemplified in the six serotonin reuptake inhibitors shown in FIG. 47. As shown in FIG. 47 these compounds have 1-2 aromatic rings, one of the rings is halogenated, there is an amino group (1°, 2°, or 3°) 3-5 carbons away from an aromatic ring and the compound topology significantly varies. For instance both alaproclate and fluvoxamine are relative linear or can adopt a linear conformation, whereas fluoxetine and zimelidine are Y-shaped and paroxetine is L-shaped. Also, sertraline have a compact fused ring system.

In spite of the structural difference among the molecules depicted in FIG. 47, these molecules bind to the same receptor site and therefore are expected to share, at least partially, receptor topology binding sites in the active site of this therapeutically important target.

One aspect of the present invention focuses on predicting membrane-binding data from structure/substructure searches and this data is used to predict ADME or other biological properties. The invention also has roots in predicting receptor binding for compounds that have an improved chance of having good ADME properties. However, predicting both receptor binding and ADME requires combining many aspects of the present invention. For instance, a set of molecules with similar membrane binding on an IAM surface may be highly diverse from a structural perspective but exhibit similar ADME. Sets of molecules with similar biological properties are special groups of compounds that can be used to discovery new attributes of biological phenomena. A set of molecules with similar membrane binding is not less important than a set of molecules with similar receptor binding. Molecules which bind to the same receptor site can be used to probe of the receptor-topology that allows compound binding. Sets of molecules that bind to the same receptor are thus valuable probes of receptor binding potential and they are even better probes, if the ADME properties of compounds can be predicted from structures.

The set of compounds shown in FIG. 47 can be considered a training set of molecules which bind to the SSRI therapeutic receptor, whereby each molecule and its surface, volume, chemical properties, etc., can be considered as probes of the active site SSRI topology when each of the ligand-molecules are bound. This suggests that substructure searching chemical libraries using each of the 6 compounds shown in FIG. 47 as query molecules will produce pools (i.e., 6 pools) of compounds with potential binding to the SSRI receptor. It is obvious that using each molecule in FIG. 47 as a query molecule for searching large databases will produce overlap between the pools, i.e., the same compound may be found by using different query molecules that bind to the same receptor. The present invention assumes that overlapping molecules or compounds that are found in multiple hit-pools obtained from using multiple query compounds in the training set (FIG. 47) have the most likely chance of binding to the SSRI receptor. Thus the objective of substructure searching with regard to finding compounds active against a receptor differs from the goal of substructure searching with regard to predicting the membrane binding properties of compounds as described above. Structure similarity searches were acceptable for predicting compound MAF values, which could then be used to predict compound ADME properties as described in Example 12, but substructure searches appeared to provide better results when trying to find a compound with both receptor binding properties and acceptable ADME properties.

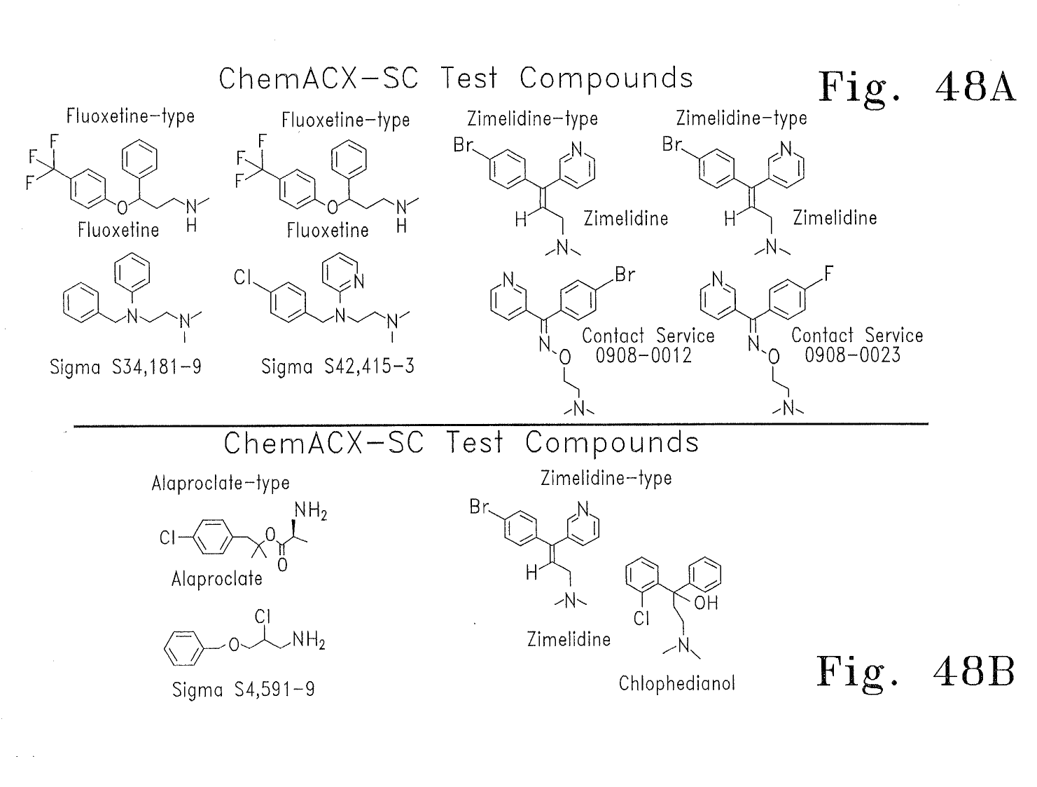

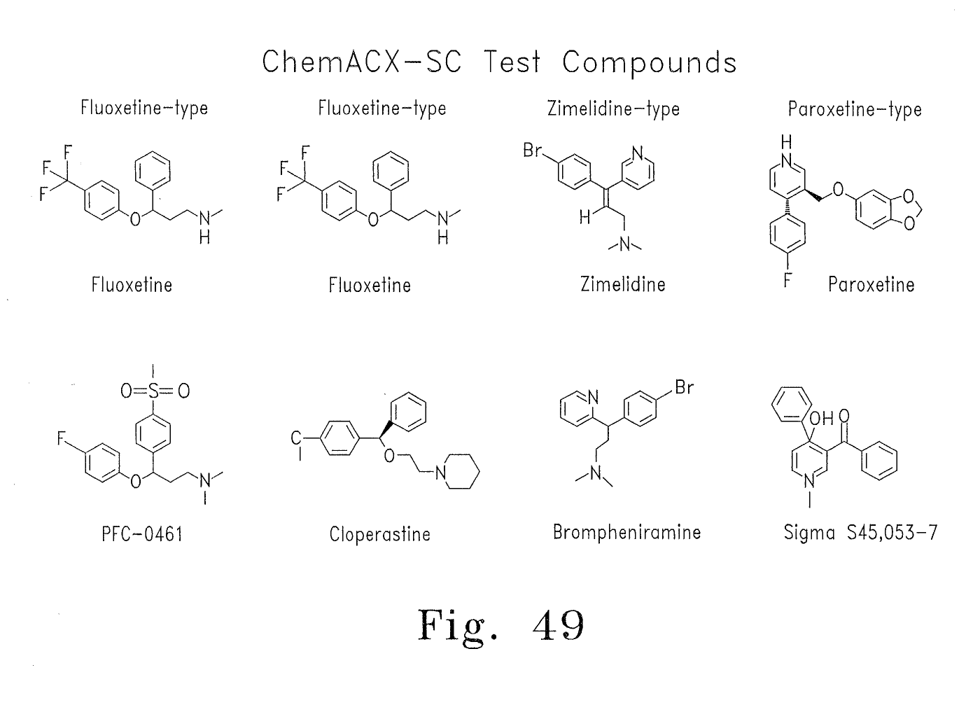

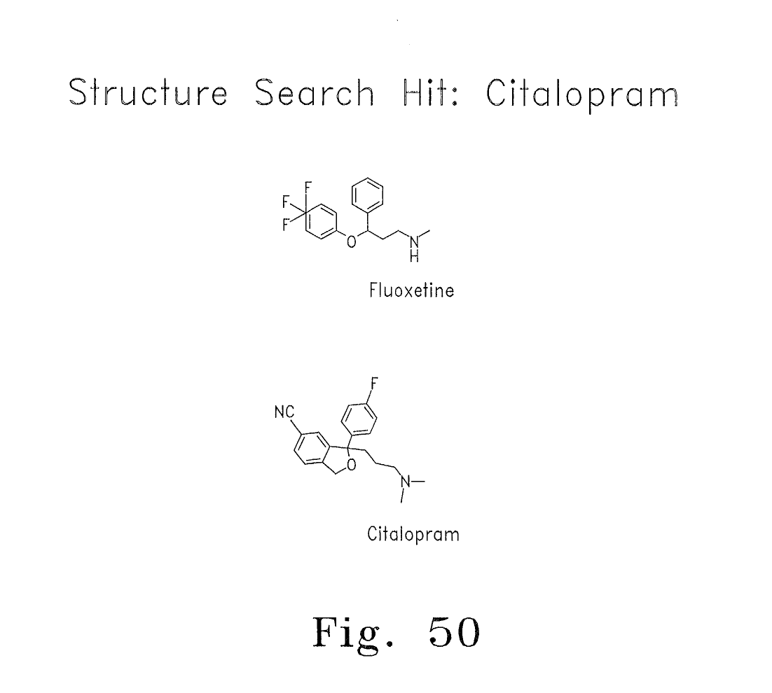

Thus each molecule shown in FIG. 47 was used to perform a substructure search of a commercial database containing ˜180,000 compounds. The substructure search included (1) combinations of fragments (e.g., aromatic ring, with or without halogen, alkylamine groups), (2) the core structure of the molecules, which represents the scaffold fit of the target (i.e., the shape of the molecule and position of the various molecular fragments in relation to one another) and (3) the molecular weight was approximately 600 or less. After searching either the Chem ACX-Pro database and the ACX-SC database approximately 1800 compounds were in a pool of compounds as potential hits. Compounds in this compound pool were eliminated based on whether they resided in MAF chemical space which means according to the ADME properties predicted from FIGS. 10, 11 and 12. This MAF filter of the compound pool allowed the compounds shown in FIGS. 48a and b to be identified as the best hits. Thus, the compounds shown in FIGS. 48a and b are believed to have the best chance of binding to the SSRI therapeutic target and also possessing ADME properties of compounds on the market that are based on this therapeutic target. The potential SSRI hits shown in FIGS. 48a and b were selected based on size, number of aromatic/saturated rings, presence of a halogenated group on an aromatic ring(s) the presence of an amino group, the position of the rings in relation to one another, the position of the amino group(s) in the molecule and how most important how well the topology of the hit compound fits that of the parent compound. Structure superimpositions were used for this last criteria, and the predicted ADME from the calibrations curves shown in FIGS. 10, 11 and 12. After searching the literature for these compounds, it was discovered that citalopram has affinity for the SSRI receptor. The other molecules do not have reported activity against SSRI based on our literature search and represent compounds with potential binding to SSRI receptors, and likely have acceptable ADME for pursuing as drug leads.

[image]

[image]

[image]

[image]

[image]

[image]

[image]

[image]

[image]

[image]

[image]

[image]

[image]

[image]

What is claimed:

1. A system for predicting pharmacokinetic properties of a proposed biologically active substance of known chemical structure based on correlation of the chemical structures of known biologically active substances, their known respective pharmacokinetic properties and their empirically defined chemical structure/membrane affinity relationships, said system comprising
a data storage device having in computer readable format a data set comprising chemical structures of a multiplicity of control compounds comprising biologically active substances, and for each compound, numeric values relating to the affinity of said compound to at least a negatively charged membrane mimetic surfaces and to a neutral membrane mimetic surface, and a numeric value relating to a known pharmacokinetic property, if any, of said compound;
a data entry device for entering the chemical structure of the proposed substance or other search parameters in computer readable format;
a programmable microprocessor in communication with said data entry device and said data storage device, said microprocessor programmed to compare the chemical structure of the proposed substance entered into the data entry device with chemical structures in the data set to identify the chemical structures in the data set having a predetermined degree of similarity with the structure of the proposed substance; and
an output device in communication with the microprocessor and capable of reporting, upon user request, the chemical structures or other identification of control compounds, if any, having the predetermined degree of similarity with the chemical structure of the proposed substance and other data stored for said identified control compound(s).

2. The system of claim 1 wherein the microprocessor is programmed to report the numeric values for the pharmacokinetic properties of the control compound(s) identified to have the predetermined degree of structural similarity.

3. The system of claim 1 wherein the microprocessor is programmed to identify control compounds having membrane binding characteristics similar to the membrane binding characteristic of the control compound(s) identified to have the predetermined degree of structure similarity to that of the proposed substance.

4. The system of claim 1 wherein the identification of control compounds having similar membrane binding characteristics is accomplished by calculating nearest neighbors to the point (X_{S}, Y_{S}) in an array of points (X_{C}, Y_{C}) wherein X_{S}, Y_{S} are the numeric values relating to the affinity of a control compound having the predetermined degree of structural similarity for a neutral membrane mimetic surface and a negatively charged membrane mimetic surface, respectively, and X_{C}, Y_{C} are the numeric values relating to the affinity of the respective control compounds for a neutral membrane mimetic surface and a negatively charged membrane mimetic surface, respectively.

5. The system of claim 2 wherein the identification of control compounds having similar membrane binding characteristics is accomplished by calculating nearest neighbors to the point (X_{S}, Y_{S}) in an array of points (X_{C}, Y_{C}) wherein X_{S}, Y_{S} are the numeric values relating to the affinity of a control compound having the predetermined degree of structural similarity for a neutral membrane mimetic surface and a negatively charged membrane mimetic surface, respectively, and X_{C}, Y_{C} are the numeric values relating to the affinity of the respective control compounds for a neutral membrane mimetic surface and a negatively charged membrane mimetic surface, respectively.

6. The system of claim 1 wherein the microprocessor is programmed to report the numeric values for the pharmacokinetic properties of the control compounds having membrane binding characteristics similar to the membrane binding characteristics of the control compound(s) having the predetermined degree of chemical structure similarity.

7. The system of claim 1 wherein the microprocessor is programmed to compare the membrane binding characteristics of the control compound(s) identified to have the predetermined degree of chemical structural similarity with an empirically defined correlation between membrane binding characteristics and numeric values for each of several pharmacokinetic properties and to report the correlation predicted numeric values for each of the pharmacokinetic properties.

8. A method for estimating the affinity of a test compound to membrane mimetic surface or a pharmacokinetic property of the test compound related to a membrane affinity, said method comprising the steps of
empirically defining a correlation between the structure of control compounds comprising drug substances and their respective affinities for membrane mimetic surfaces and preparing a data set comprising an array of control compound structures, values for their respective affinities for membrane mimetic surfaces, and values for their pharmacokinetic properties, if known;
conducting a search of the compound structures in the data set to identify control compounds having a structure with a predetermined degree of similarity with that of the test compound, and assigning the values for the membrane affinities and/or the pharmacokinetic properties of the identified control compound(s) as an estimate of the corresponding values and properties for the test compound.

9. A method for predicting pharmacokinetic properties for a proposed biologically active substance of known chemical structure, said method comprising the steps of
selecting a database including, in computer readable format, chemical structures for a multiplicity of control compounds, said control compounds comprising known biologically effective compounds and for those known effective compounds having known pharmacokinetic properties numeric values relating to at least a portion of said pharmacokinetic properties;
searching the database for compounds having a chemical structure similar to that of the proposed substance and identifying those control compounds that have a predetermined degree of similarity to the proposed substance;
identifying the pharmacokinetic properties of the control compounds having the predetermined degree of structural similarity, if any, to the proposed substance, and if no compounds are identified as having the predetermined degree of structural similarity, repeating the searching step using a lower predetermined degree of similarity until at least one control compound in the database is identified; and
reporting the pharmacokinetic properties of the identified control compound(s) to predict the pharmacokinetic properties of the proposed substance.

10. A method for designing chemical structure modifications of a hit compound found to have target receptor binding activity in vitro to produce proposed chemical structures for test substances having improved pharmacokinetic properties relative to those of said hit compound, method comprising the step of
selecting a database, including in computer readable format, structures for a multiplicity of control compounds, said compounds comprising known biologically effective substances having predetermined pharmacokinetic properties, and for each compound numeric values relating to the predetermined pharmacokinetic properties, if known, and numeric values relating to the affinity of said compound to at least a neutral membrane mimetic surface and a negatively charged membrane mimetic surface;
obtaining numeric values relating to the affinity of the hit compound to each of said membrane mimetic surfaces;
correlating the respective known pharmacokinetic properties of the control compounds with their respective positions on a plot of an array of the points (X_{A}, Y_{B}) for at least a subset of the control compounds in the database wherein X_{A} is the numeric value relating to the affinity of the compound to one membrane mimetic surface and Y_{B} is the numeric value relating to the affinity of the compound to the other membrane mimetic surface;
correlating the respective positions of the points (X_{A}, Y_{B}) in the array with chemical structural features of the respective control compounds represented by the respective points;
identifying the position of the point (H_{A}, H_{B}) in the array of points (X_{A}, Y_{B}) wherein H_{A} is the numeric value relating to the relative affinity of the hit compound to one membrane mimetic surface and H_{B} is the numeric value relating to the relative affinity of the hit compound to the other membrane mimetic surface;
identifying potential modifications of the chemical structure of the hit compound, with view of the respective correlations of chemical structure and array position and pharmacokinetic properties and array position to produce a predictable change in relative membrane binding affinities and pharmacokinetic values correlated therewith; and
preparing the compound represented by the modified chemical structure and assaying same for target macromolecule binding activity, and, optionally, membrane binding affinities.

11. A method for estimating pharmacokinetic properties of a test drug substance, said method comprising the steps of
identifying two or more membrane mimetic surfaces including a first membrane mimetic surface having a negatively charged surface and a second substantially neutral membrane mimetic surface;
identifying a set of control compounds comprising drug substances having known pharmacokinetic properties and each pharmacokinetic property being quantified for each control compound by a numeric value within a range of numeric values relating to the pharmacokinetic property;
defining for each control compound a numeric value related to its affinity for each membrane mimetic surface;
defining for the test drug substance a numeric value related to its affinity for each membrane mimetic surface
for each pharmacokinetic property, identifying a subset of control compounds having similar pharmacokinetic property related numeric values and similar membrane affinity related values to establish a correlation between a subrange of pharmacokinetic property related values and a subrange of membrane affinity related values;
comparing the membrane affinity related numeric values of the test drug substance with the correlated subranges of membrane affinity related values and pharmacokinetic property related values, and with respect to each pharmacokinetic property, identifying the subrange of numeric values related to that pharmacokinetic property correlated with the subrange of membrane affinity related numeric values for each membrane mimetic surface bracketing the respective membrane affinity related numeric values for the test drug substance; and
selecting the subrange of pharmacokinetic property related numeric values for each pharmacokinetic property best matching the membrane affinity related numeric value for the test drug substance.

12. A method for estimating pharmacokinetic properties of a test drug substance, said method comprising the steps of
identifying two or more membrane mimetic surfaces including a first membrane mimetic surface having a negatively charged surface and a second substantially neutral membrane mimetic surface;
identifying a set of control compounds comprising drug substances having known pharmacokinetic properties and each pharmacokinetic property being quantified for each control compound by a numeric value within a range of numeric values relating to the pharmacokinetic property;
defining for each control compound a numeric value related to its affinity for each membrane mimetic surface;
defining for the test drug substance a numeric value related to its affinity for each membrane mimetic surface;
for each membrane mimetic surface or for a subset of the membrane mimetic surface, establishing, if possible, a mathematical correlation of membrane affinity related numeric values for the control compounds or a subset thereof with the numeric values relating to the respective pharmacokinetic properties using said correlation to calculate estimated numerical values for the respective pharmacokinetic properties for the test drug substance.

13. A method for discovery of biologically effective compounds candidates having both potential for binding to a macromolecule recognized to be the endogenous target of a set of known effective substances and favorable pharmacokinetic properties, said method comprising the step of
identifying chemical structural or substructural characteristics of said known biologically effective substances,
conducting a structure similarity search of a database comprising chemical structures of commercially available compounds to identify structures in the database of compounds having a threshold predetermined degree of structural similarity to at least one of the known effective substances;
comparing the identified structures with those in a data set comprising the chemical structures of known effective substances and values corresponding to the relative affinity of said substances for at least two membrane mimetic surfaces and values corresponding to their pharmacokinetic properties, to eliminate, based on their probable lack of pharmacokinetic properties required for biological efficacy, at least a portion of those compounds identified to have the predetermined degree of structural similarity; and
testing at least a subset of the compounds not eliminated by the correlated comparison for binding affinity to the macromolecular target.

14. The method of claim 1 wherein the proposed biologically active substance is selected from the group consisting of a drug, a herbicide and a pesticide.

15. The method of claim 14 wherein the proposed biologically active substance is a drug.

16. The method of claim 9 wherein the proposed biologically active substance is selected from the group consisting of a drug, a herbicide and a pesticide.

17. The method of claim 16 wherein the proposed biologically active substance is a drug.

18. The method of claim 10 wherein the proposed biologically active substance is selected from the group consisting of a drug, a herbicide and a pesticide.

19. The method of claim 18 wherein the proposed biologically active substance is a drug.
